SEQUENCE LISTING (8 sequences)

SEQ ID NO: 1 - DNA, 39 bp, Artificial sequence
  Tel-1b primer
cggtttgtttgggtttgggtttgggtttgggtttgggtt

SEQ ID NO: 2 - DNA, 39 bp, Artificial sequence
  Tel-2b primer
ggcttgccttacccttacccttacccttacccttaccct

SEQ ID NO: 3 - DNA, 27 bp, Artificial sequence
  hbg1 primer
gcttctgacacaactgtgttcactagc

SEQ ID NO: 4 - DNA, 23 bp, Artificial sequence
  hbg2 primer
caccaacttcatccacgttcacc

SEQ ID NO: 5 - DNA, 23 bp, Artificial sequence
  forward primer
tttgttagcatttcagtgtttgc

SEQ ID NO: 6 - DNA, 18 bp, Artificial sequence
  reverse primer
aaggtgaaggggcaggac

SEQ ID NO: 7 - DNA, 18 bp, Artificial sequence
  foward primer
gtcctgccccttcacctt

SEQ ID NO: 8 - DNA, 18 bp, Artificial sequence
  reverse primer
agcacctcgcggtagtgg

(19) United States
(12) Patent Application Publication
(10) Pub. No.: US 2015/0005250 A1
(43) Pub. Date: Jan. 1, 2015

(54) TELOMERASE REVERSE TRANSCRIPTASE DEFICIENCY AS DIAGNOSTIC MARKER OF MYELODYSPLASTIC SYNDROME

(71) Applicant: H. LEE MOFFITT CANCER CENTER AND RESEARCH INSTITUTE, INC., TAMPA, FL (US)

(72) Inventors: Dana E. Rollison, Tampa, FL (US); Pearlie K. Burnette, Tampa, FL (US)

(21) Appl. No.: 14/349,382

(22) PCT Filed: Oct. 15, 2012
(86) PCT No.: PCT/US2012/060201

§ 371 Date: Apr. 3, 2014

Related U.S. Application Data
(60) Provisional application No. 61/546,778, filed on Oct. 13, 2011.

(51) Int. Cl. C12Q 1/68; G01N 33/573; A61K 31/454; A61K 31/706

(52) U.S. Cl. CPC C12Q 1/6883; A61K 31/706; G01N 33/573; A61K 31/454; C12Q 2600/158; G01N 2333/9128; G01N 2800/22
USPC 514/43; 506/9; 506/18; 506/16; 514/339

(57) ABSTRACT

The subject invention pertains to methods and compositions for the detection and treatment of myelodysplastic syndromes based on impaired telomerase function.

### CROSS-REFERENCE TO RELATED APPLICATION

The present application claims the benefit of U.S. Provisional Application Ser. No. 61/546,778, filed Oct. 13, 2011, which is hereby incorporated by reference herein in its entirety, including any figures, tables, nucleic acid sequences, amino acid sequences, and drawings.

### GOVERNMENT SUPPORT

This invention was made with government support under CA 129952-02 awarded by National Institutes of Health. The government has certain rights in the invention.

### BACKGROUND OF THE INVENTION

Myelodysplastic syndromes (MDS) represent a group of age-related hematological malignancies that are expanding as the world's population ages^{1, 2}. The disease is primarily characterized by impaired hematopoiesis and peripheral cytopenias³ due to diverse molecular mechanisms causing enormous heterogeneity in the disease. The early manifestations of MDS, however, are relatively well conserved and include increased apoptosis coupled to excessive proliferation of myeloid progenitors that limit the stem cell pool⁴. In response to this insult, somatic mutations or survival pathways are acquired that instill autonomous growth and clonogenic potential to the stem or progenitor cell population increasing the probability for progression to acute myeloid leukemia (AML)⁵.

Unlike the effect of acquired somatic mutations in MDS, the mechanisms contributing to apoptotic hypersensitivity within the progenitor and stem cell pool are poorly understood. T lymphocytes are dramatically altered and play a pathogenic role in this process in some patients^{6,7}. The T-cell compartment in MDS has a skewed repertoire distribution within the memory compartment and reduced numbers of naïve T-cells, an altered distribution of regulatory T-cells, and increased production of inflammatory cytokines such as tumor necrosis factor-alpha (TNF-α), Fas ligand, and other proapoptotic cytokines⁶⁻¹⁰. The idea that apoptosis in hematopoietic cells may be triggered through an immune-mediated mechanism arose from similarities with aplastic anemia (AA)^{11, 12}. Hematologic improvement in a subset of MDS patients treated with antithymocyte globulin (ATG) and cyclosporine or other immunosuppressive agents provides support for this theory¹¹⁻¹⁸. In MDS, immunosuppressive therapy response is associated with an improvement in repertoire distribution and a reduction in T-cell turnover within the naïve compartment suggesting that this treatment restores homeostasis within the T-cell compartment^{9, 15}. Although immunosuppressive therapy was initially applied in patients with hypoplastic MDS based upon overlapping features of AA, bone marrow hypocellularity has been variably associated with response^{11,14,19}. Hematologic improvement with eATG treatment in prior studies occurred preferentially in younger patients with the an HLA-DR15 class II genotype^{20,21}, but hematologic improvement in four independent studies (including the inventors' unpublished results with rabbit ATG), only occurred when this treatment was initiated early in the disease process suggesting that progressive changes in the myeloid compartment may impart immunosuppressive therapy resistance^{16,21,22}. Repertoire contraction with reduced CD4:CD8 ratio, and high lymphocyte proliferative index (PI) in immunosuppressive therapy responsive MDS patients suggested that excessive T-cell turnover may be an important risk factor contributing to autoimmunity in these patients⁹.

Progression from AA to MDS, similarities in clinical presentation and shared presentation with immune abnormalities collectively suggest there may be a common underlying pathophysiologic mechanism contributing to these diseases. Aplastic anemia is caused, in some cases, by defects in telomere biology^{23,24}. Affected hematopoietic stem cells and their progeny display accelerated telomere shortening^{23,25}. Telomeres contain hexanucleotide (TTAGGG) repeats composed of DNA and the shelterin protein complex that protects the ends of chromosomes²³. Telomeric lengthening is mediated by telomerase, which is composed of the hTERT enzyme, the TERC RNA template, and other proteins required for stabilization. In AA, several somatic and familial mutations have been identified in genes encoding elements involved in telomere repair; hTERT and TERC. Other classical telomere diseases, such as autosomal-dominant dyskeratosis congenita (DC), show similar hematopoietic cell apoptotic sensitivity, bone marrow failure, and immune deregulation, but are mediated by somatic mutations in proteins within the shelterin complex²⁶⁻²⁸ that similarly disrupt telomerase function. It is currently unclear if the immune dysregulation in AA or other telomere repair disorders are directly related to abnormal telomere biology, although a mutation occurring within a pleuripotent stem cell would be expected to affect mature T-cells.

### BRIEF SUMMARY OF THE INVENTION

Myelodysplastic syndromes (MDS) are heterogeneous disorders of dysregulated myelopoiesis with repertoire contraction and memory expansion in the peripheral T-cell compartment. Similar clinical and immune defects in aplastic anemia (AA) suggest a possible mechanistic link by an unknown mechanism. Here, the inventors show that MDS T-cells have critically short telomeres (p<0.0001), after adjustment for age and sex, and lower T-cell receptor (TCR) inducible telomerase activity (p<0.0001). Telomerase insufficiency, resulting in replication arrest, was caused by defective telomerase reverse transcriptase (hTERT) transcription in MDS T-cells and selectively affected naïve T-cells in comparison to memory cells. These results suggest that preferential loss of telomere maintenance in naïve T-cells generates a growth advantage for memory cells, which results in altered homeostatic regulation, repertoire contraction and risk for autoimmunity in this disease. Telomerase activity in MDS T-cells fell below the 95% confidence interval (MDS median=18.70, 95% CI, 15.93-20.54 vs. control median=45.00, 95% CI, 45.79-64.59) compared to controls and was unrelated to risk stratification by the International Prognostic Scoring System (IPSS) indicating that it is a frequent underlying abnormality. The inventors concluded that T-cell defects, which are functionally equivalent to AA, are mediated by telomerase insufficiency and mechanistically links MDS to other telomere disorders.

The subject invention concerns methods for detecting MDS in a subject. An MDS detection method of the invention comprises qualitatively or quantitatively analyzing a biological sample from a subject for telomerase function, wherein an impaired telomerase function is indicative of the presence of MDS in the subject. In analyzing the sample, assessment of telomerase function may be carried out using one or more methods disclosed herein, or using other methods known in the art. In some embodiments, telomerase function in CD3+ T cells within the sample are analyzed. In some embodiments, the biological sample is selected from among whole blood, plasma, or serum. In some embodiments, the biological sample comprises peripheral blood T cells (e.g., CD3+ T cells). The MDS detection method of the invention may itself be used to diagnose MDS in a subject, or the MDS detection method of the invention may be used in conjunction with other methods known in the art and used as a diagnostic aid. The subject invention also includes methods for treatment of MDS in a subject determined to have MDS using the MDS detection method of the invention.

The subject invention also concerns compositions that can be used to detect MDS. The compositions of the invention comprise an array or panel of (a) one or more binding moieties that can bind specifically to one or more proteins, or nucleic acids encoding one or more proteins, whose expression is associated with telomerase function; and (b) one or more moieties that can bind specifically to one or more proteins, or nucleic acids encoding the one or more proteins, whose expression is associated with the presence of a malignancy. Examples of binding moieties include antibodies (whole antibody or antigen-binding fragment of an antibody), peptides, nucleic acids, aptamers, and telomerase function-associated protein ligands. The malignancy may be a hematologic malignancy (such as MDS), or a non-hematologic malignancy. In some embodiments, the moieties of (a), (b), or both (a) and (b) are attached to substrate, such as a solid phase support (e.g., plate, bead, sphere).

The methods and compositions of the invention may be used to detect one or more types of MDS, such as refractory cytopenia with unilineage dysplasia (refractory anemia, refractory neutropenia, or refractory thrombocytopenia); refractory anemia with sideroblasts (SARS); refractory anemia with sideroblasts-thrombocytosis (SARS-t); refractory cytopenia with multileneage dysplasia (RCMD) (refractory cytopenia with multilineage dysplasia and ring sideroblasts (RCMD-RS), including subjects with pathological changes not restricted to red cells (i.e., prominent white cell precursor and platelet precursor (megakaryocyte) dysplasia); refractory anemia with excess blasts (RAEB)-I or -II; acute leukemia; myelodysplastic-myeloproliferative overlap syndrome; 5q-syndrome; unclassifiable myelodysplasia; and refractory cytopenia of childhood (dysplasia in childhood). In some embodiments, the MDS is one or more selected from among refractory cytopenia with multileneage dysplasia (RCMD), refractory anemia with excess blasts-1 (RAEB-1), and refractory anemia with excess blasts-2 (RAEB-2).

### BRIEF DESCRIPTION OF THE DRAWINGS

FIGS. 1A-1C: Shortened telomere length in CD3+ T cells in MDS patients. CD3+ T cells were purified by negative selection from blood of MDS patients (n=35) and healthy donors (n=42). Telomere length was detected using quantitative PCR with 293 T-cells were used as a calibrator. Results were analyzed using the ΔΔCt method. Case-control differences for the telomere lengths in CD3+ T cells were compared using the Wilcoxon signed rank test adjusting for multiple testing using the Holm's step-down method. P-value for the case-control differences are shown at the top of each panel. The correlations between telomere length and age were assessed in cases and controls using the Spearman rank correlation coefficient. P<0.05 was considered significant. FIG. 1A shows box and whisker-plots of telomere length in CD3+ T cells in MDS patients compared to controls. After adjustment for age and sex, the case-control difference for telomere lengths in CD3+ T cells was statistically significant (p<0.0001). FIG. 1B shows telomere length (y-axis) relative to hgb was inversely correlated with the age in CD3+ T cells for controls (r=−0.447, p=0.003). Although a similar trend was observed in MDS patients, the age-dependent change was not significant in MDS patients (r=−0.177, p=0.309). MDS patients and healthy donors were both divided into younger group and older group based 65 years of age. As shown in FIG. 1C, the case-control difference occurred among both age groups: the younger MDS (n=9) and younger control (n=28) (p=0.002) and the older MDS (n=26) compared to the older control group (n=14) (p=0.0026).

FIGS. 2A-2D: Impaired induction of telomerase activity in CD3+ T cells in MDS patients. CD3+ T cells were separated from peripheral blood by negative selection from MDS patients (n=35) and healthy donor (n=42) and stimulated with anti-CD3/CD28 excite beads for 3 days. Telomerase activity was measured by TRAP assay on day 0 and day 3 after stimulation (FIG. 2A). On day 0, the telomerase activity was higher in MDS patients compared to healthy donors, but on day 3 after stimulation, there was impaired induction of telomerase activity in MDS patients compared to healthy donors. Cell surface expression of CD69 in CD3+ T cell was analyzed by flow cytometry at day 0 and day 3 after stimulation (FIG. 2B). There were no statistical differences for CD69 expression in MDS patients compared to healthy donors. But on day 0, there was a trend to have higher CD69 expression in MDS T-cells compared to healthy donors (p=0.09). CD3+ T cells were negatively separated from peripheral blood of AA patients (n=8). The change in log telomerase activity, difference post-pre in stimulated T-cells was compared between the MDS, AA, and controls (FIG. 2C). Telomerase activity was impaired in both patient groups compared to healthy controls (p<0.0001). The case-control differences in telomerase activity and CD69 expression in the younger MDS (n=9) and older MDS (n=26) were compared to the younger control (n=28) and the older control (n=14) (FIG. 2D). Case-control differences for the telomerase activity in purified CD3+ T-cells were compared using the Wilcoxon signed rank test adjusting for multiple testing using the Holm's step-down method. P-value for the case-control differences are shown at the top of each panel and considered significant when P<0.05.

FIGS. 3A-3C: Telomerase insufficiency linked to defective hTERT transcription. CD3+ T-cells were purified by negative selection from blood of MDS patients (n=35) and healthy donors (n=42). hTERT mRNA expression was quantified by QRT-PCR on day 0 and day 3 after stimulation and normalized to 18s ribosomal RNA. FIG. 3A shows box and whisker plots of the relative mRNA expression on day 0 and day 3 after stimulation. The hTERT mRNA expression and telomerase activity is higher in MDS patients than healthy donors, but displayed a defect after stimulation. Differences in inducible hTERT expression in younger and older MDS patients and control show significant reduction in both groups independent of age (FIG. 3B). Inducible telomerase activity on Y-axis vs square root transformed inducible hTERT mRNA expression in MDS patients and healthy donors reveals that these two variables were strongly correlated (r=0.929489, p<0.00001) by the Pearson correlation (FIG. 3C). Case-control differences for the hTERT expression in purified CD3+ T-cells were compared using the Wilcoxon signed rank test adjusting for multiple testing using the Holm's step-down method. P-value for the case-control differences are shown at the top of each panel and considered significant when P<0.05.

FIGS. 4A-4G: Loss of naïve T-cells due to telomere deficiency. Naïve T-cells express signal joint T-cell receptor excision circles (sjTRECs), which is episomal DNA that are generated by T-cell receptor (TCR) gene rearrangement in the thymus. Since this episomal DNA fails to transfer to daughter cells, the level of TRECs in the peripheral blood is increased through thymic production and decreased by apoptosis and proliferation. CD3+ T cells were negatively separated from blood of MDS patients (n=35) and healthy donor (n=42) and the amount of signal joint TREC (sjTREC) and the +case-control differences compared in 35 MDS patients and 42 controls. sjTREC in peripheral CD3+ T-cell was detected using quantitative PCR GAPDH was used as normalization in cellular DNA (FIG. 4A). MDS patients had significantly lower TREC DNA copies per cell than healthy donors. The TREC level in MDS patients and healthy donor showed negative correlation with age due to loss of thymic function (FIG. 4B). There was a statistically significant difference in the younger MDS (n=9) and older MDS (n=26), which were compared to the younger control (n=28) and the older control (n=14), p=0.0455 and 0.0346, respectively (FIG. 4C). The phenotype of naïve (CD45RA⁺CD45RO⁻) and memory (CD45RO⁺CD45RA⁻) T-cells is shown (FIG. 4D) and are in agreement with the presence of TREC+ cells within the subpopulation of naïve T-cells by phenotypic analysis (FIG. 4E). Naïve and memory T-cells were isolated by flow sorting to 99% purity (data not shown) from MDS patients (n=8, gray box) and age-matched healthy controls (n=8, white box). DNA was extracted for telomere length analysis on day 0. The case-control difference was observed in naive T-cells (p=0.0175), but not in memory cells (p=0.3829) (FIG. 4F). Purified naive and memory T-cells from MDS and controls were stimulated with anti-CD3/CD28 beads for 3 days. Telomerase activity was measured by TRAP assay on day 3 after stimulation. Naïve T-cells in control T-cells was significantly higher than in memory cells (p=0.006) (FIG. 4G). There was impaired induction of telomerase activity in naïve T-cells (p=0.0207) in MDS relative to control T-cells, but no significant difference within the memory T-cell compartment (p=0.5054).

FIGS. 5A-5E: Deficiency of the proliferative capacity of CD3+ T cells in MDS patients. CD3+ T-cells were negatively separated from blood of MDS patients (n=35) and healthy donor (n=42) and stimulated with anti-CD3/CD28 beads for 3 days. The BrdU incorporation was measured by flow cytometry using anti-CD3 and anti-BrdU to determine the percentage of T-cells capable of entering S-phase in MDS patients and healthy donors after stimulation (FIG. 5A). A proliferation deficiency was found in MDS patients compared to healthy donors. The case-control differences of BrdU in the younger MDS (n=9) and older MDS (n=26) were compared to the younger control (n=28) and the older control (n=14) (FIG. 5B). BrdU indicates the percentage of cells capable of entering S-phase, but dilution of the number of TRECs per cell indicates the replication potential (FIG. 5C). Population doubling (PD, i.e., replication potential) was calculated as the ratio of the TREC DNA copy number per cell in unstimulated T-cells and stimulated T-cells on day 3 after antiCD3/CD28 stimulation. PD in MDS. T-cells was significantly reduced compared to healthy donors (p<0.0001). The case-control difference in PD in the younger MDS (n=9) and older MDS (n=26) compared to the younger (n=28) and older controls (n=14) indicate that the difference in replication potential is age-independent (FIG. 5D). PD was defective in T-cells with the ability to undergo S-phase transition in MDS patients (FIG. 5E). In healthy donors, the amount of PD had a significant positive association to the percentage of cells capable of entering S-phase. Case-control differences in BrdU and PD in purified CD3+ T-cells were compared using the Wilcoxon signed rank test adjusting for multiple testing using the Holm's step-down method. P-value for the case-control differences are shown at the top of each panel and considered significant when P<0.05.

FIG. 6A-6F: Deficiency of the proliferative capacity of CD3+ T-cells in MDS patients. CD3+ T-cells were negatively separated from blood of MDS patients (n=35) and healthy donors (n=42) and samples were divided into two aliquots. One aliquot was used for assays on day 0 and the other stimulated with anti-CD3/CD28 beads for 3 days. FIG. 6A shows a diagram of the analyses conducted. On day 0 cells were used for TREC baseline measurements, stained with CFSE and examined for DAPI on CD69+ cells (as described herein). On day 3, BrdU incorporation was measured by flow cytometry to determine the percentage of T-cells capable of entering S-phase in MDS patients and healthy donors after stimulation. Samples were also collected for TREC DNA content, % DAPI within the CD69+ population and for CFSE dilution assays (as described herein). BrdU indicates the percentage of cells capable of entering S-phase. The box and whisker plots of data are shown for 42 controls and 35 MDS patients (FIG. 6B). BrdU data divided into groups of younger and older patients and controls based on age <65 y and ≧65 y (FIG. 6C). Dilution of the number of TRECs per cell indicates the replication potential. Population doubling (PD, i.e., replication potential) was calculated as the ratio of the TREC DNA copy number per cell in unstimulated T-cells and stimulated T-cells on day 3 after antiCD3/CD28 stimulation. Case-control difference in PD analyzed in groups based on age <65 y and ≧65 y. Case-control differences in BrdU and PD in purified CD3+ T-cells were compared using the Wilcoxon signed rank (FIG. 6D). FIG. 6E and FIG. 6F show percentage of DAPI+CD69+ cells in unactivated T-cells at baseline (Time 0) and the percentage of these cells after activation in MDS patients and control. P-values based on Wilcoxon test for the case-control differences are shown at the top of each panel. Log-transformed values were then used in multivariate logistic regression analyses to adjust for age (as continuous variable) and sex, and the case-control. Exemplary CFSE staining in a control and case sample and summary of data in 6 donors tested is shown in supplementary FIG. 2 and confirm deficiency in population doubling. All tests were two-sided and associations were considered statistically significant at a significance level of p<0.05.

FIGS. 7A-7D: Telomere attrition within naïve T-cells. Naïve T-cells exclusively express sjTRECs and the data on sorted cell populations confirm that sjTREC DNA is present within the phenotype of naïve (CD45RA⁺CD45RO⁻) compared to memory (CD45RO⁺CD45RA⁻) T-cells (FIG. 7A). Using sjTREC expression as an indicator of the number of naïve cells in peripheral blood T-cells, bulk CD3+ T-cells from blood of MDS patients were examined. Case-control difference in TRECs were analyzed in groups based on age <65 y and ≧65 y. The same DNA was used to determine telomere length in FIGS. 1A-1C and at Day 0 for PD. sjTREC in peripheral CD3+ T-cell was detected using quantitative PCR and GAPDH was used as normalization for cellular DNA content in the sample. Naïve and memory T-cells were isolated by flow sorting to 99% purity using the gating strategy shown (FIG. 7C). Flow cytometry was conducted after gating on viable cells and gating on CD3+ T-cells. CD45RA-APC and CD45RO-PE were used for the analysis. From MDS patients (n=8, gray box) and age-matched healthy controls (n=8, white box), DNA was extracted for telomere length analysis from sorted (highly purified) naïve and memory populations (FIG. 7D). Telomere length was detected using qRT-PCR, with 293 T-cell as a calibrator, and the data was analyzed using ΔΔCt, as described herein. Case-control differences were compared using the Wilcoxon signed rank and p-values for the case-control differences are shown at the top of each panel. All tests were two-sided and associations were considered statistically significant at a significance level of p<0.05.

FIGS. 8A-8E: Impaired induction of telomerase activity in CD3+ T-cells in MDS patients. Preliminary analyses were conducted with CD3+ T-cells from healthy donors to identify the optimal time for telomerase induction using the TRAP assay on day 0, day 1, day 2, and day 3 (FIG. 8A). CD3+ T-cells were separated from peripheral blood by negative selection and unstimulated (day 0) and day 3 stimulated cells were analyzed. Stimulated cells were collated after incubation with anti-CD3/CD28-conjugated beads. The case-control differences in telomerase activity are shown for controls, MDS, aplastic anemia (AA) and large granular lymphocyte leukemia (LGLL) (FIG. 8B). CD3+ T-cells were negatively separated from peripheral blood of MDS patients (n=35) and healthy donor (n=42), AA patients (n=8) and from patients with LGL leukemia (n=17) to test for specificity. Telomerase activity (TRAP assays) and CD69 expression were compared in cases and control grouped by age <65 y and ≧65 y (FIGS. 8C and 8D). To determine that the T-cells are generally responsive to stimulation, the inventors examined the surface expression of CD69, which is a well-known early activation-associated antigen(25) by staining with PE-conjugated anti-human-CD69 and analysis by flow cytometry (FIG. 8D). From MDS patients (n=8, gray box) and age-matched healthy controls (n=8, white box), telomerase activity using TRAP assays were examined in purified sorted populations of naïve and memory cells (FIG. 8E). The sorting method and telomere length are shown in FIG. 3 for these cells. The change in telomerase activity, difference post-pre activation in T-cells is shown. hTERT activity data was normally distributed and case-control differences were compared using a T test. p-values for are shown at the top of each comparison. All tests were two-sided and associations were considered statistically significant at a significance level of p<0.05.

FIGS. 9A-9D: Telomerase insufficiency linked to defective hTERT transcription. FIG. 9A shows the region on chromosome 5q that is commonly deleted in low risk (yellow) and high risk (pink) MDS and AML (blue) as well as the position of hTERT on 5p15.33. An aliquot of the purified CD3+ T-cells used in other assays from MDS patients (n=35) and healthy donors (n=42) were used to examine inducible hTERT mRNA expression as quantified by QRT-PCR on day 0 and day 3 after stimulation normalized to 18s ribosomal RNA. FIG. 9B shows the box and whisker plots of the relative mRNA expression on day 0 and day 3 after stimulation are shown. FIG. 9C shows differences in inducible hTERT expression in younger and older groups of MDS cases and controls based on age <65 y and ≧65 y. FIG. 9D shows that inducible telomerase activity on Y-axis vs inducible hTERT mRNA expression in MDS patients (red circles) and healthy donors (black circles) reveals that these two variables were strongly correlated (r=0.89, p<0.001) by the Spearman rank correlation coefficient. Telomerase activity by the TRAP assay (data shown in FIGS. 8A-8E) was normally distributed and square-root transformed hTERT mRNA expression was used for these analyses. Results were considered significant when p<0.05. Case-control differences for the hTERT expression in purified T-cells were compared using a Wilcoxon signed rank test with p-values shown at the top of each panel.

FIGS. 10A and 10B: Correlation between proliferation defect and telomerase activity. Telomerase activity and telomere length were significantly reduced in all MDS patients relative to control but some patients retained the ability to undergo S-phase transition. Inducible telomerase activity on Y-axis (as a continuous variable) vs % BrdU in MDS patients reveals that these two variables were strongly correlated (r=0.844, p<0.0001) by the Spearman rank correlation coefficient (FIG. 10A). BrdU incorporation was not related to telomere length (as a continuous variable) in MDS patients (telomere length Y-axis vs % BrdU on X-axis) (FIG. 10B). Gray shaded area indicates the 95% CI for % BrdU in healthy controls. Results were considered significant when p<0.05.

FIGS. 11A and 11B: dilution curves for qRT-PCR and a demonstrate linear relationship of telomere length.

FIGS. 12A-12C: Reduced proliferation potential in MDS T-cells confirmed by CFSE staining. Carboxyfluorescein succinimidyl ester (CFSE) was performed according to the manufacturer's recommendations (Invitrogen, Carlsbad, Calif.). Cells were placed in a round bottom 96-well plate and stimulated with anti-CD3/anti-CD28 beads. Proliferation of T-cells was assessed by CFSE dilution after 5 days using flow cytometry on a LSRII cytometer (BD Biosciences, San Diego, Calif.) harboring a custom configuration for the H. Lee Moffitt Cancer Center and Research Institute.

FIG. 13: Telomere length shortening in LGL leukemia. Telomere length in purified T-cells was determined by methods defined for MDS and controls.

FIG. 14: Sequencing of hTERT promoter. The core promoter of hTERT was amplified with two primer pairs of 5′-TTTGTTAGCATTTCAGTGTTTGC-3′ (F1) (SEQ ID NO:5) and 5′-AAGGTGAAGGGGCAGGAC-3′ (R1) (SEQ ID NO:6) and 5′-GTCCTGCCCCTTCACCTT-3′ (F2) (SEQ ID NO:7) and 5′-AGCACCTCGCGGTAGTGG-3′ (R2) (SEQ ID NO:8). The PCR products were run on a 1% agarose gel and then cut each band from the gel separately. The final PCR products (1050 bp and 274 bp) were cloned sequenced from 5 individual MDS patients. Binding sites for c-Myc/Max/Mad (E-box), stimulatory protein 1 (SP1, GC box), MT box, Estrogen receptor responsive element (ERE), Wilm's tumor-1, (WT-1), myeloidspecific zinc finger protein 2 (MZF-2), nuclear factor of activated T-cells-1 (NFAT-1). ATG-start codon.

### DETAILED DISCLOSURE OF THE INVENTION

Based on data suggesting that immunosuppressive-therapy responsive MDS patients have accelerated turnover within the naïve T-cell compartment⁹, the inventors conduct a study to examine the replicative burst capacity and telomere function in MDS T-cells as a possible mechanism for immune dysregulation. The data herein show that T-cells from MDS and AA patients possess a similar telomere repair defect that reveals a common mechanistic link between these diseases and other primary telomere repair disorders.

Telomeres are specialized structures providing chromosome integrity during cellular division along with protection against premature senescence and apoptosis. Accelerated telomere attrition in patients with Myelodysplastic Syndrome (MDS) occurs by an undefined mechanism. Although the MDS clone originates within the myeloid compartment, T-lymphocytes display repertoire contraction and loss of naïve T-cells. The replicative lifespan of T-cells is stringently regulated by telomerase activity. In MDS cases, the inventors show that purified CD3+ T-cells have significantly shorter telomere length and reduced proliferative capacity upon stimulation compared to controls. To understand the mechanism, telomerase enzymatic activity and telomerase reverse transcriptase (hTERT) gene expression were compared in MDS cases (n=35) and healthy controls (n=42) within different T-cell compartments. Telomerase activity is greatest in naïve T-cells illustrating the importance of telomere repair in homeostatic repertoire regulation. Compared to healthy controls, MDS cases had lower telomerase induction (p<0.0001) that correlated with significantly lower hTERT mRNA (p<0.0001), independent of age and disease stratification. hTERT mRNA deficiency affected naïve but not memory T-cells, and telomere erosion in MDS occurred without evidence of an hTERT-promoter mutation, copy number variation or deletion. Telomerase insufficiency may undermine homeostatic control within the hematopoietic compartment and promote a change in the T-cell repertoire in MDS.

The subject invention concerns methods for detecting MDS in a subject. A method of the invention comprises qualitatively or quantitatively analyzing a biological sample from a subject for telomerase function, wherein an impaired telomerase function is indicative of the presence of MDS in the subject. In analyzing the sample, assessment of telomerase function may be carried out using one or more methods disclosed herein, or using other methods known in the art. In some embodiments, telomerase function in CD3+ T cells within the sample are analyzed. In some embodiments, the biological sample is selected from among whole blood, plasma, or serum. In some embodiments, the biological sample comprises peripheral blood T cells (e.g., CD3+ T cells).

Optionally, the detection method may further comprise, before or after analysis of telomerase function, additional diagnostic techniques to assess or confirm whether the subject has MDS. For example, a clinician may analyze samples of blood and bone marrow from the subject. Changes in the chromosomes of bone marrow cells (cytogenetics) may be determined.

In some embodiments, one or more of the following tests or procedures are used: physical examination (an exam of the body checking general signs of health, including checking for signs of disease, such as lumps or anything unusual) and history (history of the subject's health habits, past illnesses, and past treatments); complete blood count (CBC) with differential (blood sample is drawn and checked for the number of red blood cells and platelets, the number and types of white blood cells, the amount of hemoglobin in the red blood cells, and the portion of the blood sample made up of red blood cells); peripheral blood smear (blood sample is checked for changes in the number, type, shape, and size of blood cells, and for too much iron in the red blood cells); cytogenetic analysis (blood or bone marrow sample is viewed under the microscope to look for changes in the chromosomes); and bone marrow aspiration and biopsy (removal of bone marrow, blood, and a piece of bone by inserting a hollowing needle into the hipbone or breastbone, and the sample is viewed under a microscope to identify abnormal cells).

The subject may be symptomatic or asymptomatic of MDS at the time the methods and compositions of the invention are used. In some embodiments, the subject has been determined to be at risk of developing MDS at the time the methods and compositions of the invention are used. For example, the subject may be determined to have one, two, three, four, or five or more risk factors for MDS at the time a biological sample is obtained from the subject. Risk factors for MDS include but are not limited to prior treatment with chemotherapy (e.g., mechlorethamine (nitrogen mustard), procarbazine, chlorambucil, etoposide, teniposide, cyclophosphamide, ifosfamide, doxorubicin) with or without radiation therapy; prior treatment for Hodgkin disease, non-Hodgkin Lymphoma, or childhood acute lymphocytic leukemia; prior stem cell transplant recipient (e.g., bone marrow transplant) due to the high doses of chemotherapy typically received; presence of genetic syndromes selected from among Fanconi anemia, Shwachman-Diamond syndrome, familial platelet disorder, and severe congenital neutropenia; familial history of MDS; history of smoking tobacco; high-dose radiation exposure; exposure (e.g., inhalation, contact with skin, or consumption) to a solvent (e.g., benzene); exposure to a pesticide; exposure to a heavy metal (e.g., mercury, lead); being male, being Caucasian; and being over 60 years of age.

In some embodiments, the subject lacks one or more of the aforementioned risk factors for MDS. In some embodiments, the subject lacks all of the aforementioned risk factors for MDS.

The MDS detection method of the invention may be used to monitor the status of MDS in a subject pre-treatment and/or post-treatment. In some embodiments, the subject has previously been treated for MDS and the MDS detection method of the invention is used to monitor the status of MDS in the subject and effectiveness of treatment. Thus, one aspect of the invention is drawn to treating the subject for MDS; and detecting MDS in the subject using the method of the invention as described herein to monitor the status of the MDS and effectiveness of the treatment. In carrying out the MDS detection method of the invention, the analyzed telomerase function can be compared to a reference level of telomerase function to determine whether the measured level of telomerase function in the sample correlates with presence of the disease. When monitoring MDS in the subject, a biological sample may be obtained from the subject before, during, or immediately after MDS treatment for analysis of telomerase function (e.g., as a baseline), and one or more biological samples may be obtained subsequently and analyzed for telomerase function so that the later result(s) may be compared to the earlier result(s) to assess change in telomerase function and, thus, change in MDS status, providing information regarding treatment effectiveness.

The subject invention also includes methods for treatment of MDS in a subject, comprising detecting MDS in a subject using the detection method of the invention as described herein, and treating the subject for MDS if the subject is determined to have MDS. Thus, the treatment method comprises qualitatively or quantitatively analyzing a biological sample from a subject for telomerase function, wherein an impaired telomerase function is indicative of the presence of MDS in the subject; and treating the subject if the subject is determined to have MDS. Optionally, the treatment method may further comprise additional diagnostic techniques to confirm whether the subject has MDS.

Treatment methods for a subject with MDS depend on his or her type of MDS, risk level, age, overall health, and the subject's preferences. Treatment options include, for example, supportive care, chemotherapy, bone marrow or cord blood transplant, or other therapies, including immunosuppressive therapies such as azacitidine (Vidaza®), decitabine (Dacogen®), and lenalidomide (Revlimid®).

The subject invention also concerns compositions that can be used to detect MDS. The compositions of the invention comprise an array or panel of (a) one or more binding moieties that can bind specifically to one or more proteins, or nucleic acids encoding one or more proteins, whose expression is associated with telomerase function; and (b) one or more moieties that can bind specifically to one or more proteins, or nucleic acids encoding the one or more proteins, whose expression is associated with the presence of a malignancy. Examples of binding moieties include antibodies (whole antibody or antigen-binding fragment of an antibody), peptides, nucleic acids, aptamers, and telomerase function-associated protein ligands. The malignancy may be a hematologic malignancy (such as MDS), or a non-hematologic malignancy. In some embodiments, the moieties of (a), (b), or both (a) and (b) are attached to substrate, such as a solid phase support (e.g., plate, bead, sphere).

In the methods and compositions of the invention, the MDS may be, for example, refractory cytopenia with unilineage dysplasia (refractory anemia, refractory neutropenia, or refractory thrombocytopenia); refractory anemia with sideroblasts (SARS); refractory anemia with sideroblasts-thrombocytosis (SARS-t); refractory cytopenia with multileneage dysplasia (RCMD) (refractory cytopenia with multilineage dysplasia and ring sideroblasts (RCMD-RS), including subjects with pathological changes not restricted to red cells (i.e., prominent white cell precursor and platelet precursor (megakaryocyte) dysplasia); refractory anemia with excess blasts (RAEB)-I or -II; acute leukemia; myelodysplastic-myeloproliferative overlap syndrome; 5q-syndrome; unclassifiable myelodysplasia; or refractory cytopenia of childhood (dysplasia in childhood), based on the WHO classification criteria. In some embodiments, the MDS is RCMD, RAEB-1, or RAEB-2. The MDS may be treatment-related MDS (secondary MDS) or de novo MDS.

As used herein, the terms “telomerase function” and “telomerase activity” refer to the ability of telomerase protein components to function either in vivo or in vitro into as part of a multi-component enzyme that elongates telomeric DNA. An available assay method for detecting telomerase activity is the TRAP assay (see also the commercially available TRAP-eze telomerase assay kit (Oncor); and Morin, Cell, 1989, 59:521-529). This assay measures the amount of radioactive or non-radioactive labeled nucleotides incorporated into elongation products, polynucleotides, formed by nucleotide addition to a telomerase substrate or primer. The radioactivity or non-radioactive signals incorporated can be measured by methods well known in the art (e.g., using the PhosphorImager™ screens for radio active labels). A biological sample obtained from a subject (a test sample) and a control sample can be compared. According to the invention, a 10%, 15%, 20% 25% 30%, 40%, 50% or higher; up to 5 fold, 10 fold, 20 fold or higher difference of the telomerase activity between the test sample and the control sample indicates the modulation of telomerase activity in the test sample.

Telomerase function may be measured by values of one or more physiological parameters, such as: i) telomerase enzymatic activity; ii) telomere length; iii) cell proliferation; iv) cell senescence; and v) cell crisis. Methods for measuring values for these parameters are well known in the art and are not limited by the examples disclosed herein.

The enzymatic activity of telomerase can be measured as described herein, or by any other existing methods or equivalent methods well known in the art. By “decreased”, “deficient”, or “low” telomerase activity is meant that the absolute level of telomerase activity in the particular cell is elevated compared to normal cells in that individual, or compared to normal cells (e.g., T-cells or subsets of T-cells, such as CD3+ T-cells) in other individuals not suffering from MDS.

Human telomerase activity may be determined by measuring the rate of elongation of an appropriate repetitive sequence (primer). The activity may be measured with cytoplasmic extracts, nuclear extracts, lysed cells, whole cells, and the like (e.g., Morin, G. B. (1989) Cell, 59, 521-529). The particular sample which is employed and the manner of pretreatment will be primarily one of convenience. Depending upon the methodology used, pretreatment of the sample should be carried out under conditions which avoids denaturation of the telomerase, so as to maintain the telomerase activity.

Other techniques for measuring telomerase activity can use antibodies specific for the telomerase protein, where one may determine the amount of telomerase protein in a variety of ways. For example, one may use polyclonal antisera bound to a surface of monoclonal antibody for a first epitope bound to a surface and labeled polyclonal antisera or labeled monoclonal antibody to a second epitope dispersed in a medium, where one can detect the amount of label bound to the surface as a result of the telomerase or subunit thereof bridging between the two antibodies. Alternatively, one may provide for primers to the telomerase RNA and using reverse transcriptase and the polymerase chain reaction, determine the presence and amount of the telomerase RNA as indicative of the amount of telomerase present in the cells (e.g., CD3+ T-cells).

Procedures for measuring telomere length are known in the art and can be used in the invention (e.g., Harley, C. B., Futcher, A. B., and Greider, C. W. (1990) Nature, 345, 458-460. Levy, M. Z., Allsopp, R. C., Futcher, A. B., Grieder, C. W., and Harley, C. B. (1992) J. Mol. Biol., 225, 951-960; Lindsey, J., McGill, N. I., Lindsey, L. A., Green, D. K., and Cooke, H. J. (1991) Mutat. Res., 256, 45-48; Allsopp, R. C., Vaziri, H., Patterson, C., Goldstein, S., Younglai, E. V., Futcher, A. B., Greider, C. W., and Harley, C. B. (1992) Proc. Natl. Acad. Sci. USA, 89, 10114-10118). Typically, restriction endonuclease digestion is used (with enzymes which do not cleave telomeric DNA), and the length of the fragment having detectable telomere DNA is separated according to molecular weight by agarose gel electrophoresis. Given that the DNA sequence of a telomere is known, detection of such DNA is relatively easy by use of specific oligonucleotides.

If desired, telomeres of known length may be used as standards, whereby a determination of radioactivity may be read off a standard curve as related to telomere length. Instead, one may prepare tissues where individual cells may be assayed for relative telomere length by in situ hybridization.

The terms “cancer”, “tumor” and “malignancy” may be used interchangeably throughout the subject specification and denotes any cancerous or malignant condition, pre-cancerous lesion, myeloma, or any lymphoma or malignant condition, or any other proliferative disorder involving neoplastic cells. The terms “cancer”, “tumor”, and “malignancy” includes MDS, hematologic malignancies, non-hematologic malignancies, breast tumors, colorectal tumors, adenocarcinomas, mesothelioma, bladder tumors, prostate tumors, germ cell tumor, hepatoma/cholongio, carcinoma, neuroendocrine tumors, pituitary neoplasm, small round cell tumor, squamous cell cancer, melanoma, atypical fibroxanthoma, seminomas, nonseminomas, stromal leydig cell tumors, sertoli cell tumors, skin tumors, kidney tumors, testicular tumors, brain tumors, ovarian tumors, stomach tumors, oral tumors, bladder tumors, bone tumors, cervical tumors, esophageal tumors, laryngeal tumors, liver tumors, lung tumors, vaginal tumors and Wilm's tumor, for example.

In the detection methods of the invention, the assay is conducted to determine whether a deficient level of telomerase function is present. The phrase “deficient level” means that the absolute level of telomerase activity in the particular cell is low compared to corresponding normal cells in that individual or compared to corresponding normal cells in other individuals not suffering from MDS. By “corresponding” is meant compared to cells of the same cell type (e.g., T-cells, subpopulations of T-cells, such as CD3+ T-cells, etc.).

The detection methods of the invention can also be carried out in conjunction with other diagnostic tests. In some instances, such combination tests can provide useful information regarding the detection and progression of MDS. When the detection method is used, for example, to detect impaired telomerase function in a sample, the level of telomerase function can be used to determine the patient's status in the course of progression of the disease.

In some embodiments, the MDS detection method of the invention comprises qualitatively or quantitatively analyzing or measuring a biological sample from a subject for the presence or absence, or amount or concentration, of one or more proteins and/or nucleic acids associated with telomerase function in a subject. The analysis or measurement of the proteins and/or nucleic acid can be correlated with the status of MDS in the subject, e.g., no MDS present, MDS present, risk of MDS, effectiveness of treatment, etc. All forms of a protein, e.g., variants and fragments, such as splice variants or allelic variants, glycosylation variants, phosphorylation variants, proteolytic cleavage variants, etc., that are associated with telomerase function are contemplated within the scope of the invention. Optionally, the level of a protein or nucleic acid in a sample can be compared to a control reference standard of the same protein or nucleic acid. The methods of the invention can be used in conjunction with other assays and methodologies for screening for MDS.

The proteins can be detected and analyzed using any suitable method. In one embodiment, proteins are analyzed and detected using an antibody-based assay. Antibodies specifically reactive with a telomerase function-associated protein, or derivatives, such as enzyme conjugates or labeled derivatives, can be used to detect the telomerase function-associated protein in various biological samples, for example they may be used in any known immunoassays which rely on the binding interaction between an antigenic determinant of a protein and the antibodies. Examples of such assays are radioimmunoassay (RIA), enzyme immunoassay (e.g., ELISA), Western blotting, immunofluorescence, immunoprecipitation, latex agglutination, hemagglutination, and histochemical tests. In a further embodiment, a protein can be detected and analyzed using chromatographic techniques (e.g., HPLC, gel electrophoresis) and/or mass spectrometry (e.g., MS/MS, LC-MS/MS, GC-MS, MALDI-T of MS, SELDI-MS). In another embodiment, proteins and nucleic acids can be analyzed using standard sequencing methods known in the art.

In one embodiment, proteins associated with telomerase function can be identified, analyzed, and quantified using quantitative mass spectrometric multiple reaction monitoring (MRM) methodologies. Specific tryptic peptides can be selected as stoichiometric representatives of the proteins from which they are cleaved and quantitated against a stable isotope-labeled peptide as an internal standard to provide a measure of the concentration of the protein. The MRM methods can be coupled with procedures for enrichment of proteins such as immunodepletion and size exclusion chromatography and peptide enrichment using antibody capture (SISCAPA).

An antibody specific for the protein associated with telomerase function can be labeled with a detectable substance (i.e., a detectable label) and localized in biological samples based upon the presence of the detectable substance. Examples of detectable substances include, but are not limited to, the following radioisotopes (e.g., ³H, ¹⁴C, ³⁵S, ¹²⁵I, ¹³¹I), fluorescent labels (e.g., FITC, rhodamine, lanthanide phosphors), quantum dots (e.g., Qdot nanocrystals are nanometer size atom clusters containing atoms of a semiconductor material (e.g., cadmium mixed with selenium or tellurium) which has been coated with an additional semiconductor shell (e.g., zinc oxide)), luminescent labels such as luminol; enzymatic labels (e.g., horseradish peroxidase, beta-galactosidase, luciferase, alkaline phosphatase, acetylcholinestease), biotinyl groups (which can be detected by marked avidin, e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags). Indirect methods may also be employed in which the primary antigen-antibody reaction is amplified by the introduction of a second antibody, having specificity for the antibody reactive against the telomerase function-associated protein. By way of example, if the antibody having specificity against the telomerase function-associated protein is a rabbit IgG antibody, the second antibody may be goat anti-rabbit gamma-globulin labeled with a detectable substance as described herein.

Methods for conjugating or labeling the antibodies discussed above may be readily accomplished by one of ordinary skill in the art. Time-resolved fluorometry may be used to detect a signal.

Therefore, in accordance with one embodiment of the invention, a method is provided wherein an antibody to a protein associated with telomerase function is labeled with an enzyme, a substrate for the enzyme is added wherein the substrate is selected so that the substrate, or a reaction product of the enzyme and substrate, forms fluorescent complexes with a lanthanide metal. A lanthanide metal is added and the protein associated with telomerase function is quantitated in the sample by measuring fluorescence of the fluorescent complexes. Antibodies specific for the telomerase function-associated protein may be directly or indirectly labeled with an enzyme. Enzymes are selected based on the ability of a substrate of the enzyme, or a reaction product of the enzyme and substrate, to complex with lanthanide metals such as europium and terbium. Examples of suitable enzymes include alkaline phosphatase and beta-galactosidase. Preferably, the enzyme is alkaline phosphatase. Antibodies may also be indirectly labeled with an enzyme. For example, the antibodies may be conjugated to one partner of a ligand binding pair, and the enzyme may be coupled to the other partner of the ligand binding pair. Representative examples include avidin-biotin, and riboflavin-riboflavin binding protein. Preferably the antibodies are biotinylated, and the enzyme is coupled to streptavidin.

In an embodiment of the method, antibody bound to a telomerase function-associated protein in a sample is detected by adding a substrate for the enzyme. The substrate is selected so that in the presence of a lanthanide metal (e.g., europium, terbium, samarium, and dysprosium, preferably europium and terbium), the substrate or a reaction product of the enzyme and substrate, forms a fluorescent complex with the lanthanide metal. Examples of enzymes and substrates for enzymes that provide such fluorescent complexes are described in U.S. Pat. No. 5,312,922 to Diamandis. By way of example, when the antibody is directly or indirectly labeled with alkaline phosphatase, the substrate employed in the method may be 4-methylumbeliferyl phosphate, or 5-fluorosalicyl phosphate. The fluorescence intensity of the complexes can be measured, for example, using a time-resolved fluorometer, e.g., a CyberFluor 615 Immunoanalyzer (Nordion International, Kanata Ontario).

The sample or the antibody specific for the telomerase function-associated protein may be immobilized on a carrier. Examples of suitable carriers are agarose, cellulose, dextran, Sephadex, Sepharose, liposomes, carboxymethyl cellulose polystyrene, filter paper, ion-exchange resin, plastic film, plastic tube, glass beads, polyamine-methyl vinyl ether-maleic acid copolymer, amino acid copolymer, ethylene-maleic acid copolymer, nylon, silk, etc. The carrier may be in the shape of, for example, a tube, test plate, well, beads, disc, sphere, etc. The immobilized antibody may be prepared by reacting the material with a suitable insoluble carrier using known chemical or physical methods, for example, cyanogen bromide coupling.

In accordance with one embodiment, the present invention provides a mode for determining a protein associated with telomerase function in a sample by measuring the protein by immunoassay. It will be evident to a skilled artisan that a variety of immunoassay methods can be used to measure the telomerase function-associated protein. In general, an immunoassay method may be competitive or noncompetitive. Competitive methods typically employ an immobilized or immobilizable antibody to the telomerase function-associated protein and a labeled form of the telomerase function-associated protein. The telomerase function-associated protein in the sample and the labeled telomerase function-associated protein compete for binding to the antibody. After separation of the resulting labeled telomerase function-associated protein that has become bound to the antibody (bound fraction) from that which has remained unbound (unbound fraction), the amount of the label in either bound or unbound fraction is measured and may be correlated with the amount of the telomerase function-associated protein in the biological sample in any conventional manner, e.g., by comparison to a standard curve.

A noncompetitive method can also be used for the determination of a teloermase function-associated protein, with the most common method being the “sandwich” method. In this assay, two antibodies, both of which bind to the telomerase function-associated protein, are employed. One of the antibodies is directly or indirectly labeled (also referred to as the “detection antibody”) and the other antibody is immobilized or immobilizable (also referred to as the “capture antibody”). The capture and detection antibodies can be contacted simultaneously or sequentially with the biological sample. Sequential methods can be accomplished by incubating the capture antibody with the sample, and adding the detection antibody at a predetermined time thereafter (sometimes referred to as the “forward” method); or the detection antibody can be incubated with the sample first and then the capture antibody added (sometimes referred to as the “reverse” method). After the necessary incubation(s) have occurred, to complete the assay, the capture antibody is separated from the liquid test mixture, and the label is measured in at least a portion of the separated capture antibody phase or the remainder of the liquid test mixture. Generally, it is measured in the capture antibody phase since it comprises the telomerase function-associated protein bound by (“sandwiched” between) the capture and detection antibodies.

In a typical two-site immunometric assay for a telomerase function-associated protein, one or both of the capture and detection antibodies are polyclonal antibodies. The label used in the detection antibody can be selected from any of those known conventionally in the art. As with other embodiments of the protein detection assay, the label can be an enzyme or a chemiluminescent moiety, for example, or a radioactive isotope, a fluorophore, a quantum dot, a detectable ligand (e.g., detectable by a secondary binding by a labeled binding partner for the ligand), and the like. Preferably, the antibody is labeled with an enzyme that is detected by adding a substrate that is selected so that a reaction product of the enzyme and substrate forms fluorescent complexes. The capture antibody is selected so that it provides a mode for being separated from the remainder of the test mixture. Accordingly, the capture antibody can be introduced to the assay in an already immobilized or insoluble form, or can be in an immobilizable form, that is, a form which enables immobilization to be accomplished subsequent to introduction of the capture antibody to the assay. An immobilized capture antibody can comprise an antibody covalently or noncovalently attached to a solid phase such as a magnetic particle, a latex particle, a microtiter multi-well plate, a bead, a cuvette, or other reaction vessel. An example of an immobilizable capture antibody is an antibody that has been chemically modified with a ligand moiety, e.g., a hapten, biotin, or the like, and that can be subsequently immobilized by contact with an immobilized form of a binding partner for the ligand, e.g., an antibody, avidin, or the like. In one embodiment, the capture antibody can be immobilized using a species specific antibody for the capture antibody that is bound to the solid phase.

A particular sandwich immunoassay method of the invention employs two antibodies reactive against a telomerase function-associated protein, a second antibody having specificity against an antibody reactive against the telomerase function-associated protein labeled with an enzymatic label, and a fluorogenic substrate for the enzyme. In one embodiment, the enzyme is alkaline phosphatase (ALP) and the substrate is 5-fluorosalicyl phosphate. ALP cleaves phosphate out of the fluorogenic substrate, 5-fluorosalicyl phosphate, to produce 5-fluorosalicylic acid (FSA). 5-Fluorosalicylic acid can then form a highly fluorescent ternary complex of the form FSA-Tb(3+)-EDTA, which can be quantified by measuring the Tb³⁺ fluorescence in a time-resolved mode. Fluorescence intensity is typically measured using a time-resolved fluorometry as described herein.

The above-described immunoassay methods and formats are intended to be exemplary and are not limiting since, in general, it will be understood that any immunoassay method or format can be used in the present invention.

Depending upon the protein, expression of a protein associated with telomerase function in a subject having MDS can be lowered or deficient as compared to expression of that same protein in a normal subject that does not have MDS. Similarly, expression of a protein associated with telomerase function in a subject having MDS can increase as compared to expression of that same protein in a normal subject that does not have MDS. The increase or decrease in expression can be viewed as a ratio of protein expression levels in normal subjects versus subjects having MDS. As used herein, a ratio of protein expression level having a positive value represents that the particular protein is found at elevated levels in a subject having MDS as compared to a subject that does not have MDS. A ratio of protein expression level having a negative value represents that the particular protein is found at lower levels in a subject having MDS as compared to a subject that does not have MDS. In one embodiment, where the level of expression of a particular protein is elevated in a subject having MDS, the ratio for the particular protein can be about 1.3 or greater, or about 1.5 or greater, or about 2.0 or greater, or about 4.0 or greater. In one embodiment, where the level of expression of a particular protein is decreased in a subject having MDS, the ratio, for the particular protein can be about −1.3 or less, −1.5 or less, or −2.0 or less.

The presence or amount of a protein associated with a subject having MDS can be compared to a reference control for that protein to determine if the level of protein corresponds to the level of the protein typically found in a normal subject or to the level of the protein typically found in a subject with MDS. For example, if the level of a protein associated with MDS in a patient having MDS is about twice the level of the same protein in a patient that does not have MDS, then a biological sample to be assayed can be analyzed for the presence and level of the protein and compared against a reference control level of that protein in a normal subject and/or in a subject having MDS.

Nucleic acids include naturally occurring nucleic acids, oligonucleotides, antisense oligonucleotides, and synthetic oligonucleotides that hybridize to the nucleic acid encoding a telomerase function-associated protein. The present invention contemplates the use of nucleic acid sequences corresponding to the coding sequence of the protein associated with telomerase function and to the complementary sequence thereof, as well as sequences complementary to the transcript sequences occurring further upstream or downstream from the coding sequence (e.g., sequences contained in, or extending into, the 5′ and 3′ untranslated regions) for use as agents for detecting the expression of the protein in biological samples of MDS patients, or those at risk of MDS.

The preferred oligonucleotides for detecting the presence of nucleic acid encoding a protein associated with telomerase function in biological samples are those that are complementary to at least part of an RNA or DNA sequence encoding the protein. Oligonucleotides may be oligoribonucleotides or oligodeoxyribonucleotides. In addition, oligonucleotides may be natural oligomers composed of the biologically significant nucleotides, i.e., A (adenine), dA (deoxyadenine), G (guanine), dG (deoxyguanine), C (cytosine), dC (deoxycytosine), T (thymine) and U (uracil), or modified oligonucleotide species, substituting, for example, a methyl group or a sulfur atom for a phosphate oxygen in the inter-nucleotide phosohodiester linkage. Additionally, these nucleotides themselves, and/or the ribose moieties may be modified.

The oligonucleotides may be synthesized chemically, using any of the known chemical oligonucleotide synthesis methods well described in the art. For example, the oligonucleotides can be prepared by using any of the commercially available, automated nucleic acid synthesizers. Alternatively, the oligonucleotides may be created by standard recombinant DNA techniques, for example, inducing transcription of the noncoding strand. The DNA sequence encoding the protein may be inverted in a recombinant DNA system, e.g., inserted in reverse orientation downstream of a suitable promoter, such that the noncoding strand now is transcribed.

Although any length oligonucleotide may be utilized to hybridize to a nucleic acid encoding a protein associated with telomerase function, oligonucleotides typically within the range of 8-100 nucleotides are generally used. In one embodiment, oligonucleotides for use in detecting a telomerase function-associated protein can be within the range of 15-50 nucleotides.

An oligonucleotide selected for hybridizing to a nucleic acid molecule, whether synthesized chemically or by recombinant DNA technology, can be isolated and purified using standard techniques and then optionally labeled (e.g., with ³⁵S or ³²P) using standard labeling protocols.

The present invention also contemplates the use of oligonucleotide pairs in polymerase chain reactions (PCR) to detect a nucleic acid encoding a telomerase function-associated protein of the invention in biological samples. The oligonucleotide pairs can include a forward primer and a reverse primer.

The presence of a nucleic acid encoding a protein associated with telomerase function in a sample from a patient may be determined by nucleic acid hybridization, such as but not limited to Northern blot analysis, dot blotting, Southern blot analysis, fluorescence in situ hybridization (FISH), and PCR. Chromatography, such as HPLC, and other known assays may also be used to determine messenger RNA levels in a sample.

In one aspect, the present invention contemplates the use of nucleic acids as agents for detecting telomerase function-associated proteins in biological samples of patients, wherein the nucleic acids are labeled. The nucleic agents may be labeled with a radioactive label, a fluorescent label, a quantum dot, an enzyme, a chemiluminescent tag, a colorimetric tag or other labels or tags that are discussed above or that are known in the art.

In another aspect, the present invention contemplates the use of Northern blot analysis to detect the presence of telomerase function-associated protein mRNA in a biological sample. The first step of the analysis involves separating a sample containing nucleic acid by gel electrophoresis. The dispersed nucleic acids are then transferred to a nitrocellulose filter or another filter. Subsequently, the filter is contacted with labeled oligonucleotide under suitable hybridizing conditions, e.g., 50% formamide, 5×SSPE, 2×Denhardt's solution, 0.1% SDS at 42° C., as described in Molecular Cloning: A Laboratory Manual, Maniatis et al. (1982, CSH Laboratory). Other useful procedures known in the art include solution hybridization, dot and slot RNA hybridization, and probe based microarrays. Measuring the radioactivity of hybridized fragments, using standard procedures known in the art quantitates the amount of a particular nucleic acid present in the biological fluid of a patient.

Dot blotting involves applying samples that may contain a nucleic acid of interest to a membrane. The nucleic acid can be denatured before or after application to the membrane. The membrane is incubated with a labeled probe. Dot blot procedures are well known to the skilled artisan and are described more fully in U.S. Pat. Nos. 4,582,789 and 4,617,261, the disclosures of which are incorporated herein by reference.

Polymerase chain reaction (PCR) is a process for amplifying one or more specific nucleic acid sequences present in a nucleic acid sample using primers and agents for polymerization and then detecting the amplified sequence. The extension product of one primer when hybridized to the other becomes a template for the production of the desired specific nucleic acid sequence, and vice versa, and the process is repeated as often as is necessary to produce the desired amount of the sequence. PCR is routinely used to detect the presence of a desired sequence (U.S. Pat. No. 4,683,195).

A specific example of PCR that is routinely performed by the skilled artisan to detect desired sequences is reverse transcription PCR (RT-PCR; Saiki et al. (1985) and Scharf et al. (1986)). RT-PCR involves isolating total RNA from biological fluid, denaturing the RNA in the presence of primers that recognize the desired nucleic acid sequence, using the primers to generate a cDNA copy of the RNA by reverse transcription, amplifying the cDNA by PCR using specific primers, and detecting the amplified cDNA by electrophoresis or other methods known to the skilled artisan. The amount of a target nucleic acid sequence in a sample can be quantitated using standard PCR methods.

In one embodiment, the detection method of the present invention is used to detect, diagnose, and/or monitor therapy of MDS. The methods of the invention can be used to establish a prognosis and/or to design, determine, and/or monitor therapeutic treatments on a subject having MDS. For example, the presence or levels of telomerase function-associated proteins or nucleic acids in a subject can be monitored prior to treatment, such as supportive care, chemotherapy, bone marrow or cord blood transplant, or other immunosuppressive therapy, and/or monitored during and after a treatment regimen is completed. The detection methods of the invention can also be used to monitor for remission or relapse of a subject.

The results obtained from using a method of the invention can be recorded on a tangible medium and/or reported to the subject. The results obtained can also be used by a clinician to manage therapeutic treatments and protocols that the patient may receive.

In some embodiments, the subject exhibits no symptoms of MDS at the time a method of the invention is carried out. In other embodiments, the subjects exhibit one or more symptoms of MDS at the time a method of the invention is carried out. For example, with respect to MDS, the one or more symptoms may include symptoms caused by low numbers of blood cells:
Red blood cells carry oxygen throughout the body. Low numbers can lead to anemia—feeling tired or weak, being short of breath and looking pale. Anemia is the most common symptom of MDS.
White blood cells fight infection. Low numbers can lead to fever and frequent infections.
Platelets control bleeding. Low numbers can lead to easy bleeding or bruising.

The subject invention also concerns compositions that can be used to detect telomerase function-associated proteins that are differentially expressed in subjects having MDS as compared to subjects that do not have MDS. In one embodiment, a composition of the invention comprises one or more isolated telomerase function-associated proteins, or nucleic acid encoding them, which can optionally be provided as part of an array, panel, container, etc. In another embodiment, a composition of the invention comprises a panel or array of antibodies, or antigen binding fragments thereof, which can specifically bind to a telomerase function-associated protein. The antibodies can be monoclonal or polyclonal antibodies. Antigen binding fragments include, but are not limited to, F(ab′)2, Fab′, Fab, and Fv. In another embodiment, a composition of the invention comprises a panel or array of peptides or nucleic acids (e.g., aptamers) that can specifically bind to a protein associated with telomerase function. In another embodiment, a composition of the invention comprises a panel or array of ligands that can bind specifically to a protein associated with telomerase function. For example, if the telomerase function-associated protein is a receptor protein, the ligand can be the natural biological ligand that binds to the receptor protein or a synthetic ligand that has been designed to bind to the receptor protein. Binding moieties of the invention, such as antibodies, peptides, and aptamers, that can bind to a telomerase function-associated protein can be prepared using standard methods and materials in the art, or may be commercially available. Compositions of the invention can be provided on a solid phase support, such as plastic or nitrocellulose. Compositions of the invention can also include a reference control for one or more telomerase function-associated proteins wherein a predetermined amount of the protein is provided. For example, a reference control protein can be provided such that if the protein is present in a sample, the level or amount of the protein present can be compared to the level or amount of the same protein typically found in a subject having MDS and/or a subject that does not have MDS.

In one aspect, the composition of the present invention is a kit comprising required elements for diagnosing or monitoring MDS. In one embodiment, the kits comprise a container for collecting a biological sample from a patient and an agent for detecting and/or quantifying the presence of a telomerase function-associated protein or nucleic acid encoding it. The components of the kits can be packaged either in aqueous medium or in lyophilized form.

The methods of the invention can be carried out using a diagnostic kit for qualitatively or quantitatively detecting a telomerase function-associated protein or encoding nucleic acid in a sample such as blood. By way of example, the kit can contain binding agents (e.g., antibodies) specific for a telomerase function-associated protein, antibodies against the antibodies labeled with an enzyme; and a substrate for the enzyme. The kit can also contain a solid support such as microtiter multi-well plates, standards, assay diluent, wash buffer, adhesive plate covers, and/or instructions for carrying out a method of the invention using the kit. In one embodiment, the kit includes one or more protease inhibitors (e.g., a protease inhibitor cocktail) to be applied to the biological sample to be assayed (such as blood).

Kits for diagnosing or monitoring MDS containing one or more agents that detect a telomerase function-associated protein, such as but not limited to antibodies, or fragments thereof, or other binding moiety, can be prepared. The agent(s) can be packaged with a container for collecting the biological fluid from a patient. When the antibodies or binding moiety are used in the kits in the form of conjugates in which a label is attached, such as a radioactive metal ion or a moiety, the components of such conjugates can be supplied either in fully conjugated form, in the form of intermediates or as separate moieties to be conjugated by the user of the kit.

Kits containing one or more agents that detect nucleic acid encoding telomerase function-associated protein, such as but not limited to the full length nucleic acid, oligonucleotides, and pairs of primers can also be prepared. The agent(s) can be packaged with a container for collecting biological samples from a patient. The nucleic acid can be in the labeled form or to be labeled form.

Other components of the kit may include but are not limited to, means for collecting biological samples, means for labeling the detecting agent (binding agent), membranes for immobilizing the telomerase function-associated protein or nucleic acid in the biological sample, means for applying the biological sample to a membrane, means for binding the agent to the telomerase function-associated protein or nucleic acid in the biological sample of a subject, a second antibody, a means for isolating total RNA from a biological fluid of a subject, means for performing gel electrophoresis, means for generating cDNA from isolated total RNA, means for performing hybridization assays, and means for performing PCR, etc.

As used herein, the term “ELISA” includes an enzyme-linked immunoabsorbent assay that employs an antibody or antigen bound to a solid phase and an enzyme-antigen or enzyme-antibody conjugate to detect and quantify the amount of an antigen or antibody present in a sample. A description of the ELISA technique is found in Sites et al. (1982) and in U.S. Pat. Nos. 3,654,090; 3,850,752; and 4,016,043, the disclosures of which are herein incorporated by reference. ELISA is an assay that can be used to quantitate the amount of antigen, proteins, or other molecules of interest in a sample. In particular, ELISA can be carried out by attaching on a solid support (e.g., polyvinylchloride) an antibody specific for an antigen or protein of interest. Cell extract or other sample of interest such as urine can be added for formation of an antibody-antigen complex, and the extra, unbound sample is washed away. An enzyme-linked antibody, specific for a different site on the antigen is added. The support is washed to remove the unbound enzyme-linked second antibody. The enzyme-linked antibody can include, but is not limited to, alkaline phosphatase. The enzyme on the second antibody can convert an added colorless substrate into a colored product or can convert a non-fluorescent substrate into a fluorescent product. The ELISA-based assay method provided herein can be conducted in a single chamber or on an array of chambers and can be adapted for automated processes.

In these exemplary embodiments, the antibodies can be labeled with pairs of FRET dyes, bioluminescence resonance energy transfer (BRET) protein, fluorescent dye-quencher dye combinations, beta gal complementation assays protein fragments. The antibodies may participate in FRET, BRET, fluorescence quenching or beta-gal complementation to generate fluorescence, colorimetric or enhanced chemiluminescence (ECL) signals, for example.

These methods are routinely employed in the detection of antigen-specific antibody responses, and are well described in general immunology text books.

The methods and compositions of the present invention can be used with humans and other animals of any age or gender. The other animals contemplated within the scope of the invention include domesticated, agricultural, or zoo- or circus-maintained animals. Domesticated animals include, for example, dogs, cats, rabbits, ferrets, guinea pigs, hamsters, pigs, monkeys or other primates, and gerbils. Agricultural animals include, for example, horses, mules, donkeys, burros, cattle, cows, pigs, sheep, and alligators. Zoo- or circus-maintained animals include, for example, lions, tigers, bears, camels, giraffes, hippopotamuses, and rhinoceroses. In some embodiments, the subject is a mammal (human or non-human).

Biological samples refer to a composition obtained from a human or animal. Biological samples within the scope of the invention include, but are not limited to, whole blood, blood plasma, serum, urine, tears, saliva, sputum, exhaled breath, nasal secretions, pharyngeal exudates, bronchoalveolar lavage, tracheal aspirations, interstitial fluid, lymph fluid, meningal fluid, amniotic fluid, glandular fluid, feces, perspiration, mucous, vaginal or urethral secretion, cerebrospinal fluid, and transdermal exudate. A biological sample also includes experimentally separated fractions of all of the preceding solutions or mixtures containing homogenized solid material, such as feces, tissues, and biopsy samples.

Samples and/or binding moieties may be arrayed on a solid support, or multiple supports can be utilized, for multiplex detection or analysis. “Arraying” refers to the act of organizing or arranging members of a library (e.g., an array of different samples), or other collection, into a logical or physical array. Thus, an “array” refers to a physical or logical arrangement of, e.g., biological samples. A physical array can be any “spatial format” or physically gridded format” in which physical manifestations of corresponding library members are arranged in an ordered manner, lending itself to combinatorial screening. For example, samples corresponding to individual or pooled members of a sample library can be arranged in a series of numbered rows and columns, e.g., on a multi-well plate. Similarly, binding moieties can be plated or otherwise deposited in microtitered, e.g., 96-well, 384-well, or -1536 well, plates (or trays). Optionally, binding moieties may be immobilized on the solid support.

Detection of proteins associated with telomerase function, and nucleic acids encoding them, and other assays that are to be carried out on samples, can be carried out simultaneously or sequentially, and may be carried out in an automated fashion, in a high-throughput format.

As used herein, the terms “link”, “join”, or “attach” refer to any method known in the art for functionally connecting moieties and molecules, such as peptides and nucleic acids, including, without limitation, recombinant fusion, covalent bonding, disulfide bonding, ionic bonding, hydrogen bonding, and electrostatic bonding. For example, the compositions of the invention include one or more moieties that can bind specifically to one or more proteins, or nucleic acids encoding the one or more proteins, whose expression is associated with telomerase function. Such moieties can be attached to a substrate, such as a solid phase support.

As used herein, the terms solid “support”, “substrate”, and “surface” refer to a solid phase which is a porous or non-porous water insoluble material that can have any of a number of shapes, such as strip, rod, particle, beads, or multi-welled plate. In some embodiments, the support has a fixed organizational support matrix that preferably functions as an organization matrix, such as a microtiter tray. Solid support materials include, but are not limited to, cellulose, polysaccharide such as Sephadex, glass, polyacryloylmorpholide, silica, controlled pore glass (CPG), polystyrene, polystyrene/latex, polyethylene such as ultra high molecular weight polyethylene (UPE), polyamide, polyvinylidine fluoride (PVDF), polytetrafluoroethylene (PTFE; TEFLON), carboxyl modified teflon, nylon, nitrocellulose, and metals and alloys such as gold, platinum and palladium. The solid support can be biological, non-biological, organic, inorganic, or a combination of any of these, existing as particles, strands, precipitates, gels, sheets, pads, cards, strips, dipsticks, test strips, tubing, spheres, containers, capillaries, pads, slices, films, plates, slides, etc., depending upon the particular application. Preferably, the solid support is planar in shape, to facilitate contact with a biological sample such as urine, whole blood, plasma, serum, peritoneal fluid, or ascites fluid. Other suitable solid support materials will be readily apparent to those of skill in the art. The solid support can be a membrane, with or without a backing (e.g., polystyrene or polyester card backing), such as those available from Millipore Corp. (Bedford, Mass.), e.g., HI-FLOW Plus membrane cards. The surface of the solid support may contain reactive groups, such as carboxyl, amino, hydroxyl, thiol, or the like for the attachment of nucleic acids, proteins, etc. Surfaces on the solid support will sometimes, though not always, be composed of the same material as the support. Thus, the surface can be composed of any of a wide variety of materials, such as polymers, plastics, resins, polysaccharides, silica or silica-based materials, carbon, metals, inorganic glasses, membranes, or any of the aforementioned support materials (e.g., as a layer or coating).

As used herein, the terms “label” and “tag” refer to substances that may confer a detectable signal, and include, but are not limited to, enzymes such as alkaline phosphatase, glucose-6-phosphate dehydrogenase, and horseradish peroxidase, ribozyme, a substrate for a replicase such as QB replicase, promoters, dyes, quantum dots, fluorescers, such as fluorescein, isothiocynate, rhodamine compounds, phycoerythrin, phycocyanin, allophycocyanin, o-phthaldehyde, and fluorescamine, chemiluminescers such as isoluminol, sensitizers, coenzymes, enzyme substrates, radiolabels, particles such as latex or carbon particles, liposomes, cells, etc., which may be further labeled with a dye, catalyst or other detectable group.

As used in this specification, the singular forms “a”, “an”, and “the” include plural reference unless the context clearly dictates otherwise. Thus, for example, a reference to “an antibody” includes more than one such antibody. A reference to “a molecule” includes more than one such molecule, and so forth.

### EXEMPLIFIED EMBODIMENTS

### Embodiment 1

A method for detecting myelodysplastic syndrome (MDS) in a subject, comprising analyzing a biological sample from the subject for telomerase function, wherein an impaired telomerase function is indicative of MDS in the subject.

### Embodiment 2

The method according to Embodiment 1, wherein the analyzing comprises determining one or more of the following: TERT transcription (TERT mRNA expression), telomere length, telomerase activity, TERC RNA template, telomerase induction (inducible TERT level), and T-cell receptor excision circles (TREC).

### Embodiment 3

The method according to Embodiment 1 or 2, further comprising obtaining the biological sample from the subject.

### Embodiment 4

The method according to any preceding Embodiment, wherein the biological sample is whole blood, plasma, or serum.

### Embodiment 5

The method according to any preceding Embodiment, wherein the biological sample comprises peripheral blood T cells.

### Embodiment 6

The method according to any preceding Embodiment, wherein the biological sample comprises CD3+ T cells, and the analyzing comprises analyzing telomerase function in the CD3+ T cells.

### Embodiment 7

The method according to any preceding Embodiment, wherein the MDS is one or more of among refractory cytopenia with unilineage dysplasia (refractory anemia, refractory neutropenia, or refractory thrombocytopenia); refractory anemia with sideroblasts (SARS); refractory anemia with sideroblasts-thrombocytosis (SARS-t); refractory cytopenia with multileneage dysplasia (RCMD) (refractory cytopenia with multilineage dysplasia and ring sideroblasts (RCMD-RS), including subjects with pathological changes not restricted to red cells (i.e., prominent white cell precursor and platelet precursor (megakaryocyte) dysplasia); refractory anemia with excess blasts (RAEB)-I or -II; acute leukemia; myelodysplastic-myeloproliferative overlap syndrome; 5q-syndrome; unclassifiable myelodysplasia; or refractory cytopenia of childhood (dysplasia in childhood).

### Embodiment 8

The method according to any preceding Embodiment, wherein the MDS is refractory cytopenia with multilineage dysplasia (RCMD), refractory anemia with excess blasts-1 (RAEB-1), or refractory anemia with excess blasts-2 (RAEB-2).

### Embodiment 9

The method according to any preceding Embodiment, wherein the subject is determined to be at risk of developing MDS.

### Embodiment 10

The method according to any preceding Embodiment, wherein the analyzing comprises measuring telomerase function in the biological sample and comparing the measured level of telomerase function to a reference level of telomerase function.

### Embodiment 11

The method according to any preceding Embodiment, wherein the subject has not previously been treated for the MDS.

### Embodiment 12

The method according to any of Embodiments 1 to 10, wherein the subject has previously been treated for MDS and telomerase function is analyzed to monitor the status of MDS in the subject and/or effectiveness of the treatment.

### Embodiment 13

A method for treatment of myelodysplastic syndrome (MDS) in a subject determined to have MDS, comprising administering a therapeutic treatment to the subject, wherein the subject has been determined to have MDS based on impaired telomerase function.

### Embodiment 14

The method according to Embodiment 12, wherein the method comprises qualitatively or quantitatively analyzing a biological sample from the subject for telomerase function, wherein an impaired telomerase function is indicative of the presence of MDS in the subject; and treating the subject if the subject is determined to have MDS.

### Embodiment 15

The method according to Embodiment 12 or 13, wherein the analyzing comprises determining one or more of the following: TERT transcription (TERT mRNA expression), telomere length, telomerase activity, TERC RNA template, telomerase induction (inducible TERT level), and T-cell receptor excision circles (TREC).

### Embodiment 16

The method according to any preceding Embodiment, wherein the biological sample is whole blood, plasma, or serum.

### Embodiment 17

The method according to any preceding Embodiment, wherein the biological sample comprises peripheral blood T cells.

### Embodiment 18

The method according to any preceding Embodiment, wherein the biological sample comprises CD3+ T cells, and the analyzing comprises analyzing telomerase function in the CD3+ T cells.

### Embodiment 19

The method according to any preceding Embodiment, wherein the MDS is one or more of among refractory cytopenia with unilineage dysplasia (refractory anemia, refractory neutropenia, or refractory thrombocytopenia); refractory anemia with sideroblasts (SARS); refractory anemia with sideroblasts-thrombocytosis (SARS-t); refractory cytopenia with multilineage dysplasia (RCMD) (refractory cytopenia with multilineage dysplasia and ring sideroblasts (RCMD-RS), including subjects with pathological changes not restricted to red cells (i.e., prominent white cell precursor and platelet precursor (megakaryocyte) dysplasia); refractory anemia with excess blasts (RAEB)-I or -II; acute leukemia; myelodysplastic-myeloproliferative overlap syndrome; 5q-syndrome; unclassifiable myelodysplasia; or refractory cytopenia of childhood (dysplasia in childhood).

### Embodiment 20

The method according to any preceding Embodiment, wherein the MDS is refractory cytopenia with multilineage dysplasia (RCMD), refractory anemia with excess blasts-1 (RAEB-1), or refractory anemia with excess blasts-2 (RAEB-2).

### Embodiment 21

The method according to any preceding Embodiment, further comprising obtaining a biological sample from the subject after the treating; and analyzing the biological sample for telomerase function, wherein an impaired telomerase function is indicative of MDS status in the subject.

### Embodiment 22

A method for monitoring myelodysplastic syndrome (MDS) in a subject post-treatment, comprising administering a treatment for the MDS to the subject; and analyzing a biological sample from the subject for telomerase function, wherein the biological sample is obtained from the subject post-treatment, and wherein an impaired telomerase function in the biological sample is indicative of MDS in the subject.

### Embodiment 23

A composition comprising an array or panel for detection of myelodysplastic syndrome (MDS), comprising: (a) one or more moieties that can bind specifically to one or more proteins, or nucleic acids encoding the one or more proteins, whose expression is associated with telomerase function.

### Embodiment 24

The composition of Embodiment 23, further comprising: (b) one or more moieties that can bind specifically to one or more proteins, or nucleic acids encoding the one or more proteins, whose expression is associated with the presence of a malignancy.

### Embodiment 25

The composition of Embodiment 23 or 24, wherein the one or more moieties of (a) and/or (b) comprises one or more moieties selected from among an antibody, or an antigen binding fragment of the antibody, a peptide, a nucleic acid, or a ligand.

### Embodiment 26

The composition of Embodiment 24 or 25, wherein the malignancy is a hematologic malignancy.

### Embodiment 27

The composition of Embodiment 26, wherein the hematologic malignancy is MDS.

### Embodiment 28

The composition of any preceding Embodiment, further comprising a substrate, wherein the one or more moieties of (a) and (b) are attached to the substrate.

### Embodiment 29

The composition of Embodiment 28, wherein the substrate is a solid phase support.

### Embodiment 30

The composition of any preceding Embodiment, wherein the one or more moieties of (a) and/or (b) further comprises a detectable label attached thereto.

The practice of the present invention can employ, unless otherwise indicated, conventional techniques of molecular biology, microbiology, recombinant DNA technology, electrophysiology, and pharmacology that are within the skill of the art. Such techniques are explained fully in the literature (see, e.g., Sambrook, Fritsch & Maniatis, Molecular Cloning: A Laboratory Manual, Second Edition (1989); DNA Cloning, Vols. I and II (D. N. Glover Ed. 1985); Perbal, B., A Practical Guide to Molecular Cloning (1984); the series, Methods In Enzymology (S. Colowick and N. Kaplan Eds., Academic Press, Inc.); Transcription and Translation (Hames et al. Eds. 1984); Gene Transfer Vectors For Mammalian Cells (J. H. Miller et al. Eds. (1987) Cold Spring Harbor Laboratory, Cold Spring Harbor, N.Y.); Scopes, Protein Purification Principles and Practice (2nd ed., Springer-Verlag); and PCR: A Practical Approach (McPherson et al. Eds. (1991) IRL Press)), each of which are incorporated herein by reference in their entirety.

Experimental controls are considered fundamental in experiments designed in accordance with the scientific method. It is routine in the art to use experimental controls in scientific experiments to prevent factors other than those being studied from affecting the outcome.

All patents, patent applications, provisional applications, and publications referred to or cited herein, supra or infra, are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Materials and Methods for Examples 1-7

Patients and Healthy Controls.

MDS and AA patients were studied using peripheral blood samples collected through the Rare Disease Clinical Research Network involving four centers: Hematologic Malignancy Program at the Moffitt Cancer Center (“Moffitt”), the Cleveland Clinic Cancer Center, UCLA Medical Center and Penn State Cancer Center. MDS (n=35) and AA (n=8) cases were histologically-confirmed and classified according to both World Health Organization (WHO)^{40,41} and International Prognostic Scoring System (IPSS) criteria³. The MDS patients were classified by IPSS as low-risk (n=12), intermediate-1 (n=9), intermediate-2 (n=8), and high-risk (n=6). All study procedures were approved by the institutional review board (IRB) at the participating institutions and, all study participants signed informed consent for acquisition of peripheral blood in sodium heparin tubes. The clinical and hematological features of the MDS group are summarized in Table 1. For controls (n=42), buffy coats were obtained from healthy donors, ages 17 to 84 years old, that were donors at the Southwest Florida Blood Services, St Petersburg, Fla. Peripheral blood mononuclear cells were isolated from blood or buffy coats by Ficoll-Hypaque (Amersham Pharmacia Biothech, Piscataway, N.J., USA) gradient centrifugation, as described previously, and frozen in liquid nitrogen⁹. Purified CD3+ T-cells were isolated using negative selection (Stemcell technologies, Vancouver, BC, Canada) immediately prior to conducting functional experiments. Briefly, the enrichment cocktail was added to PBMCs at 50 μl/ml and incubated 20 min at room temperature and centrifugation was used to separate the CD3+ T-cell population. These purified CD3+ T-cells were then stimulated in RPMI 1640 medium (Gibco, Grand Island, N.Y.) containing 10% FBS with CD3/CD28 T-cell activator beads (Dynabead®, Invitrogen, Carlsbad, Calif., USA) for 3 days.

TABLE 1
Clinical characteristics of myelodysplastic
syndrome (MDS) cases

| Characteristic | |
|---|---|
| Age | 70 (28-87) |
| Sex | |
| Male | 24 |
| Female | 11 |
| APSS¹ score | |
| Low | 12 |
| Intermediate-1 | 9 |
| Intermediate-2 | 8 |
| High | 6 |
| Cytogenetics | |
| Normal | 15 |
| Abnormal | 20 |
| WHO² MDS subtype | |
| refractory cytopenia with | 13 |
| multilineage dysplasia (RCMD) | |
| RA with ringed sideroblasts (RARS) | 2 |
| RCMD with ringed sideroblasts (RCMD-RS) | 2 |
| RA with excess blasts (RAEB)-1 | 5 |
| RAEB-2 | 9 |
| MDS-unclassfied (MDS-U) | 4 |

¹IPSS = International Prognostic Scoring System;
²WHO = World Health Organization

Measurement of Telomere Length.

DNA was extracted from CD3+ T-cells using the PureLink Genomic DNA Kits according to manufacturer's instructions (Invitrogen, Carlsbad, Calif.), and relative telomere lengths were measured by a modified version of the quantitative real-time (qRT) PCR-based telomere assay, as described previously^{29,30}. This method has been used in several previous case-control studies of telomere length and cancer^{42,43}. Briefly, the telomere repeat copy number to single gene copy number (T/S) ratio was determined using an Applied Biosystems (Foster City, Calif.) 7900 HT PCR system in a 96-well format. Ten ng of genomic DNA was used for either the telomere or hemoglobin (hgb) PCR reaction and the primers used were as follows: Tel-1b primer (CGG TTT GTT TGG GTT TGG GTT TGG GTT TGG GTT TGG GTT) (SEQ ID NO:1), Tel-2b primer (GGC TTG CCT TAC CCT TAC CCT TAC CCT TAC CCT TAC CCT) (SEQ ID NO:2), hbg1 primer (GCTTCTGACACAACTGTGTTCACTAGC) (SEQ ID NO:3), and hbg2 primer (CACCAACTTCATCCACGTTCACC) (SEQ ID NO:4). All samples were analyzed by both the telomere and hemoglobin reactions and the analysis was performed in triplicate on the same plate. In addition to the test samples, each 96-well plate contained a five-point standard curve from 0.08 ng to 50 ng using genomic DNA. The standard curve in each plate was run to assess interassay variations in PCR efficiency. The T/S ratio (-dCt) for each sample was calculated by subtracting the median hemoglobin threshold cycle (Ct) value from the median telomere Ct value. The relative T/S ratio (-ddCt) was determined by subtracting the T/S ratio of the 10.0 ng standard curve point from the T/S ratio of each unknown sample.

Measurement of TREC in CD3+ T Cells.

Genomic DNA was extracted from CD3+ T cell, as described above. To detect signal joint (sj) TRECs, a real-time quantitative PCR method was used⁴⁴. Each PCR reaction was performed in triplicate in a 25-μl solution containing 250 ng DNA, 1.0× TaqMan Universal PCR master mix (Applied Biosystems, Branchburg, N.J.), 200 nM forward and reverse primer, and 250 nM probe. Quantification of TREC was normalized by GAPDH. GAPDH was detected in 100 ng of DNA using 300 nM of the forward primer, 900 nM of the reverse primer, and 875 nM of the probe. In addition to the samples, a five-point standard curve of sjTREC from 1×10 copies to 1×10⁵ copies and a five-point standard curve of GAPDH from 1×10³ copies to 1×10⁷ copies were used to quantify the results. The standard curve in each plate was run to assess interassay variations in PCR efficiency. Quantitative PCR was performed by an ABI system (PerkinElmer), a standard curve was plotted, and TREC values were calculated by the ABI770 software. Samples were analyzed in triplicate and sjTREC levels expressed as copies per 250 ng of genomic DNA.

Measurement of hTERT mRNA Expression.

Total RNA was extracted from CD3+ T cells using the RNeasy Mini Kit (Qiagen, Chatsworth, Calif.). Reverse transcription was performed using the high-capacity cDNA reverse transcription kit (Applied Biosystems) in accordance with the manufacturer's suggestions. Relative hTERT gene expression was measured by a qRT-PCR with 18S rRNA used as a reference gene. Expression levels of hTERT mRNA and 18S rRNA were evaluated with Taqman Probes obtained from ABI. The reaction mixture consisted of 1× Taqman mixture, 1× primer of hTERT or 18S rRNA. All samples for both the hTERT and 18S rRNA were tested in triplicate in the same plate. The expression level of hTERT for each sample was calculated with the ΔΔCt method.

Telomerase Activity.

Telomerase activity was determined using the telomerase PCR-ELISA kit (TRAP) (Roche Molecular Biochemicals, Indianapolis, Ind., USA) according to the manufacturer's instructions. In brief, an equal amount (2×10⁵ cells) of lysis extract from each sample was incubated with a biotinylated telomerase substrate oligonucleotide (P1-TS primer) at 25° C. for 30 minutes. At the same time, a heat-treated (85° C. for 10 minutes) negative control was included for each sample during incubation. Next, the extended products were amplified using PCR with P1-TS primers. The PCR program consisted of 30 cycles at 95° C. for 30 sec, 50° C. for 30 sec, and 72° C. for 90 sec, followed by 72° C. for 10 min. After 10 minutes of denaturation, the PCR-amplified products for each sample were hybridized with buffer T and buffer IS for 2 hours, respectively, and immobilized onto a streptavidin-coated microplate. Negative controls were only treated with hybridization buffer T. The resulting products are then detected with the anti-DIG-HRP and detected with TMB substrate solution. The absorbance of the final colored reaction was measured at 450 nm with a reference wavelength of approximate 690 nm in a microtiter plate reader (Bio-Rad, Hercules, Calif., USA). For each plate, one positive control was set as a calibrator in order to standardize between different runs. Experiments were carried out at least twice to improve the reliability of the results. The relative telomerase activity within each sample was calculated as follows: (the absorbance of the sample—the absorbance of the heat-treated sample)/the absorbance of the internal standard of the sample.

Flow Cytometry to Detect BrdU Incorporation.

Basal cell proliferation in unstimulated cells was determined by Ki67 nuclear antigen expression. Ki67 is expressed by cells in late G1, S, G2 and M phase of the cell cycle and reflect the in vivo proliferative state. Anti-Ki67-PE was used to stain the intracellular nuclear antigen after subsequent fixation and permeabilization (BD Biosciences Fix and Perm kit, San Jose, Calif., USA), as recommended by the manufacture and as shown previously⁹. To access entry into S-phase, T-cells were analyzed after TCR stimulation for 3 days with antiCD3/antiCD28 using bromodeoxyuridine (BrdU) incorporation (BrdU flow kit, BD Biosciences, San Diego, Calif.). As a control for TCR responsiveness, the inventors examined the surface expression of CD69, which is a well-known recent activation-associated antigen^{45,46}. For all proliferation assays, 10 μM of BrdU was added during the last 45 min of CD3+ T cell activation. BrdU-pulsed CD3+ T-cells were harvested and stained with PE-conjugated anti-human-CD69. The surface stained cells were then fixed and permeabilized with BD Cytofix/Cytoperm buffer, treated with staining enhancer Cytoperm plus buffer and re-fixed, followed by incubation with DNase for 1 hour at 37° C. to expose the BrdU. After washing with Cytoperm buffer, intracellular BrdU was stained with fluorescent anti-BrdU-FITC antibody for 20 min at room temperature. Finally, cells were resuspended with staining buffer that contained 3% Fetal Bovine Serum (FBS). The percentage of cells that incorporated BrdU and expressed CD69 was determined on a FACScan flow cytometer (BD Biosciences) using FlowJo software (BD Biosciences).

Naïve and memory cell purification. T-cells subsets were purified from CD3+ T-cells using CD45RO and CD45RA antibody staining and isolation with the Aria flow cytometry (BD Biosciences) sorter and TREC expression was shown to reside within the CD45RA+/CD45RO− naïve population consistent with being a recent thymic emigrant^{47,48}. Subset purity was >95% in post-sort analyses. Memory (CD45RO+CD45RA⁻) or naïve (CD45RA+CD45RO−) cells were stimulated with CD3/CD28 Dynabeads (Dynal) for 3 days. Telomere length was detected in naïve and memory T-cells before stimulation and telomerase activity and hTERT expression was measured using the TRAP assay and qRT-PCR reaction, respectively, before and after the 3 days of stimulation.

Statistical Analysis.

Statistical analysis was performed with the SPSS 19.0 software package (SPSS Inc., Chicago, Ill., USA). The data were analyzed using linear regression to evaluate correlation with age. The Wilcoxon rank sum test was used as a non-parametric test to assess case-control differences. To adjust for age and sex, multivariable logistic regression was used. All tests were two-sides and associations were considered statistically significant at a significance level of p<0.05. The specific statistical tests that were used in each experiment are noted in the figure and table legends. Since T-cells are suspected to having a direct effect on the hematopoiesis of younger MDS patients under the age of 65 years old, statistical analyses were performed in patients and controls divided into 2 groups based on age using 65 years as the cutpoint. Results were compared in 28 younger controls (<65 y, median 44, range 17-63 years) and 9 younger patients (median 57, range 28-61 years). Fourteen controls (≧65 y, median 71.5, range 65-84) and 26 MDS patients (median 72, range 65-87) comprised an older group for statistical comparisons.

### Example 1

### Clinical Characteristics of MDS and AA Patients

Characteristics of the 35 MDS patients and 8 AA patients are summarized in Table 1. Patient ages ranged from 28 to 87 with a mean age of 70 years. Among the MDS patients, patients were classified as refractory cytopenia with multilineage dysplasia with or without ringed sideroblasts (RCMD and RCMD-RS) (n=4), refractory anemia with excess blasts (RAEB)-1 (n=5) and RAEB-2 (n=9) based on the WHO classification criteria. Based on IPSS, 21 (60%) were lower risk (low or intermediate-1) and 14 (40%) were higher risk (intermediate-2 or high risk). Fifteen of 35 (42.8%) had identifiable cytogenetic abnormalities by metaphase karytyping or FISH and 20 (57.2%) had normal cytogenetics.

### Example 2

### Shorter Telomere Length in Purified CD3+ T Cells in MDS Patients Compared to Controls

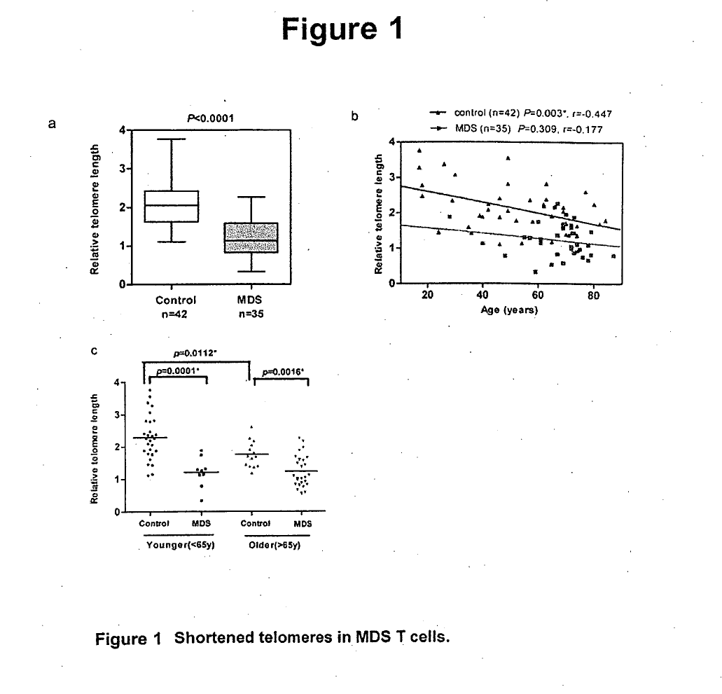

Telomere length in MDS and (n=35) and control (n=42) T-cells were compared in unstimulated cells. The relative mean telomere length relative to hgb was significantly shorter among MDS patients (median=1.133, 95% CI, 1.061-1.389) compared to controls (median=2.053, 95% CI, 1.908-2.321) after adjustment for age and sex (p<0.0001) (FIG. 1A). In healthy individuals, T-cell telomere length declined progressively with age (r=−0.447, p=0.003) (FIG. 1B) in agreement with previous reports^{32,49}. Age was also inversely correlated with telomere length in MDS T-cells, but this trend was not statistically significant (r=−0.177, p=0.309) (FIG. 1B). To further evaluate the effect of age on telomere length shortening in MDS, the difference between telomere length in younger (age <65 yrs) and older (≧65 yrs) groups of healthy donors and MDS patients was compared. As shown in FIG. 1C, telomere attrition was a normal feature of aging since there was telomere shortening in the older control group compared to the younger control group (p=0.0112). Comparing age-matched cases and control groups (younger group, p=0.0002 and older group, p=0.0026) significant telomere shortening occurred independent of the age group indicating that age is not responsible for the telomere shortening in T-cells from MDS patients.

### Example 3

### Telomerase Enzyme Activity in CD3+ T-Cells Fails to be Induced Upon TCR Stimulation in MDS Patients

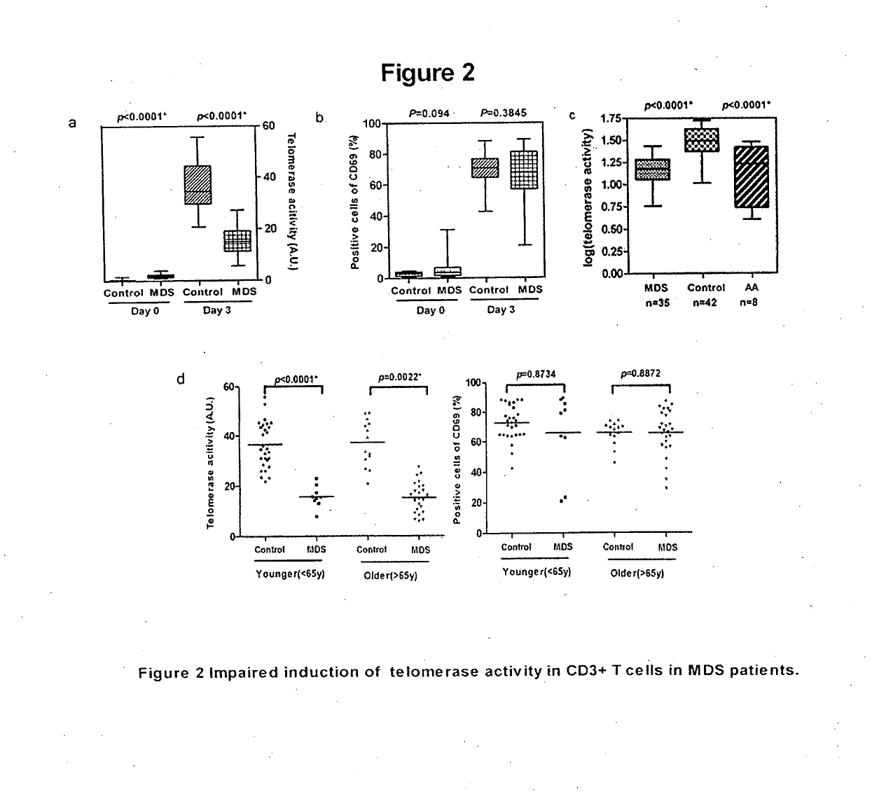

Shorter telomere-length in T-cells suggests that like myeloid cells, the T-cells from MDS may have been exposed to excessive proliferative stress or may have an inherent defect in telomere repair. To assess the telomerase repair function in MDS T-cells, the TCR was stimulated with antiCD3/antiCD28 excite beads to induce telomerase and the amount of activity was determined by the TRAP assay. Results were compared at day 0 (i.e., basal activity) and after 3 days of stimulation (i.e., inducible activity). The 3-day stimulation period was found to be optimal for telomerase activation in control cells (data not shown). At day 0, the basal level of telomerase activity was up-regulated in MDS patients compared to healthy controls (FIG. 2A, p<0.0001), although this may be below the threshold to initiate telomere repair. The inducible telomerase activity on day 3 of stimulation was increased above basal levels in both patients and controls. (FIG. 2A), but MDS T-cells were significantly less effective (median=17.04, 95% CI, 15.35-18.08) compared to healthy controls (median=33.60, 95% CI, 32.63-39.33) (p<0.0001) at inducing telomerase activity (FIG. 2A).

To ensure that T-cells from patients were adequately responsive to TCR stimulation, the inventors measured the surface expression of CD69, which is an inducible early activation antigen (FIG. 2B)⁴⁶. In contrast to telomerase activity, the expression of CD69 was similar in cases and controls on day 3 (FIG. 2B, p=0.3845) suggesting that intracellular signaling through the TCR is intact and that the defect in telomerase activity is specific. On day 0, the expression of CD69 was slightly higher in MDS patients compared to healthy donors, but this was not statistical significance (p=0.09), possibly reflecting a greater number of in vivo antigen-activated cells.

Aplastic anemia (AA) and MDS are thought to arise from a similar pathophysiological mechanism in some cases. Some aplastic anemia patients harbor congenital or somatically-acquired mutations in telomerase components, hTERT and hTERC, within the hematopoietic stem cell compartment that limits their telomere repair pathway. Although the prominent immune defects that occur in this disease have not been attributed to telomere defects, the inventors measured the inducible telomerase activity and observed a significant reduction in both MDS and AA T-cells after 3 days of stimulation, as shown in FIG. 2C.

The difference between the younger and older groups of MDS patients and controls was then compared to assess the impact of age on inducible telomerase activity. A significant case-control difference was observed in both age groups (FIG. 2C). In contrast to telomere length which decreased over time with age in controls, telomerase activity was well maintained in the older control group suggesting that telomere shortening normally occurs despite having adequate inducible telomerase enzyme activity (FIG. 2D): Inducible CD69 expression showed no statistical difference among the age-matched groups of patients and controls indicating that there was no difference in TCR-mediated response although two younger patients appeared to be hyporesponsive in the younger group.

### Example 4

### hTERT Transcriptional Deficiency Responsible for Impaired Telomerase Function

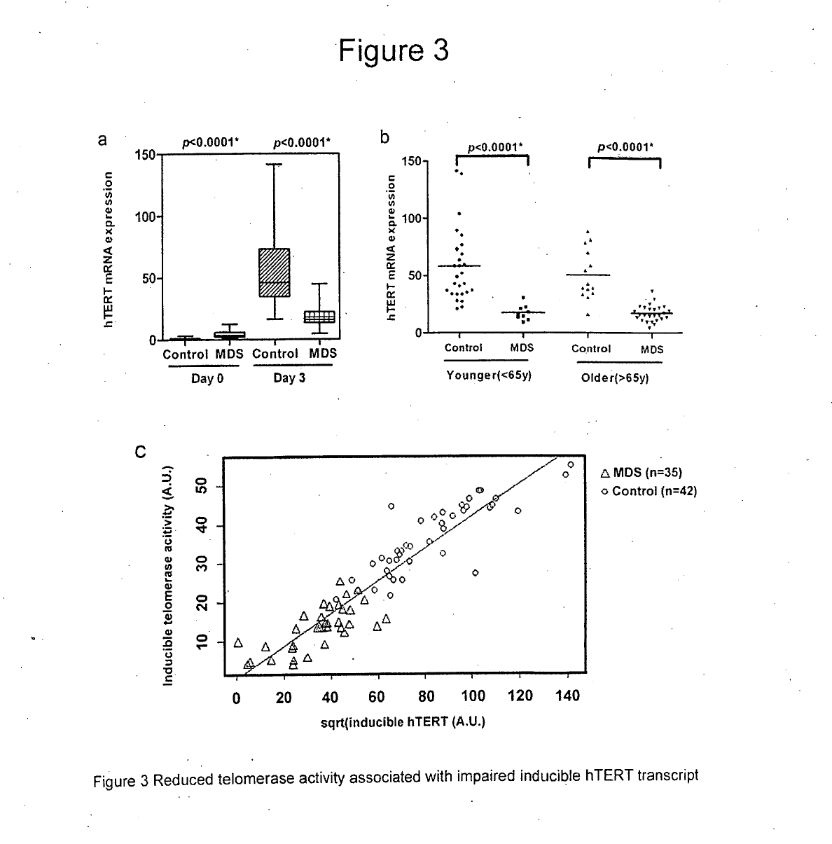

Telomerase activity was reduced in MDS and AA T-cells. In AA, the defect may be due to genetic mutations within the stem cell compartment. At least 9 distinct mutations in hTERT and TERC have been identified, but the inventors' previous report showed that these mutations are not present in MDS despite short telomeres in the myeloid compartment and B lymphocytes, although the telomere shortening in B cells was not statistically significant³⁰. In T-cells, activation of hTERT mRNA represents the rate-limiting for step for telomerase induction. Therefore, the basal level of hTERT and inducible hTERT, after 3-days of TCR stimulation, were compared in MDS and control T-cells. As shown in FIG. 3A, basal hTERT expression was up-regulated in MDS patients compared to healthy controls (p<0.0001), which may reflect the presence of more activated cells in vivo. Inducible hTERT transcription was significantly lower in MDS patients (median=18.70, 95% CI, 15.93-20.54) compared to healthy controls (median=45.00, 95% CI, 45.79-64.59) (p<0.0001) (FIG. 3A) and this case-control difference occurred in among younger and older groups (FIG. 3B). The amount of inducible hTERT mRNA expression was then correlated to the level of inducible telomerase activity and found to strongly positively associated, as shown in FIG. 3C. These results suggest that the telomerase deficiency in MDS is mediated by an hTERT transcriptional defect. Interestingly, it is evident that all MDS patients fall below the 95% CI of controls with regard to hTERT and telomerase function indicating that this is a severe defect in T-cells from all MDS patients.

### Example 5

### Defective Telomere Regulation in NaïVe T-Cells in MDS Patients

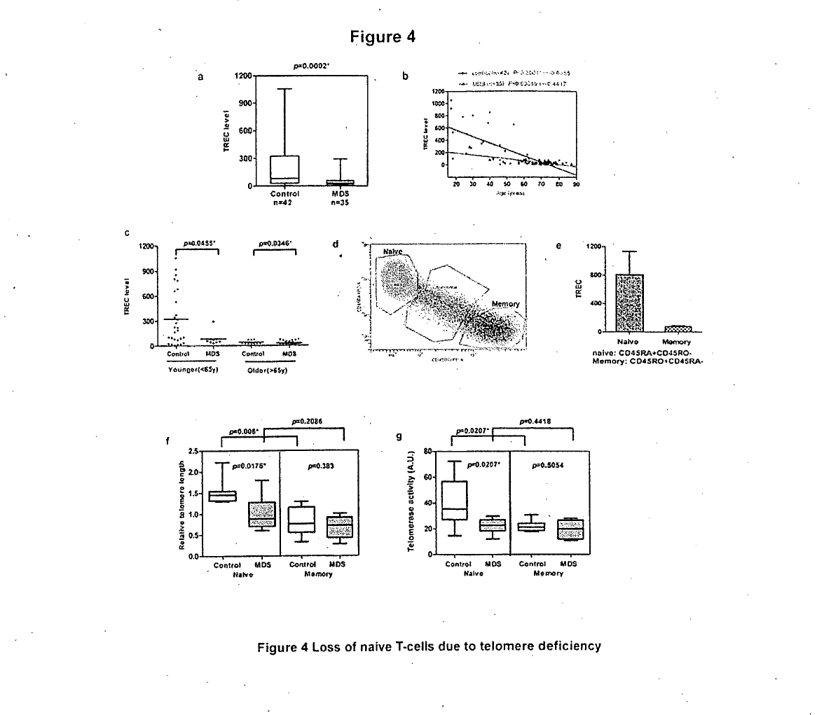

Telomerase induction is necessary to avoid apoptosis in the T-cell compartment during homeostatic or antigen-induced proliferation⁴⁵. Not only is the percentage of cells induced to proliferate important, but the clonal burst size is also critical. The maintenance of naïve T-cells is essential to prevent premature aging within the T-cell compartment and to maintain repertoire diversity³⁴. Although the phenotype of naïve T-cells is controversial, the expression of T-cell receptor excision circles (TRECs) appears only in recent thymic emigrants because these episomal DNA fragments, which are generated from TCR gene rearrangement, fail to transfer to daughter cells^{47,48}. TRECs are therefore found exclusively in naïve T-cells⁵⁰. The amount of TREC-expression reflects both thymic output and proliferative history in peripheral T-cells. In unstimulated cells, the amount of TREC DNA was markedly reduced in MDS patients (31.99, 95% CI, 27.5-61.0) compared to healthy controls (80.56, 95% CI, 137.7-322.2) (p=0.0002) (FIG. 4A) and the amount of TREC DNA declined with age in both groups (FIG. 4B), confirming the loss of naïve TREC+ cells through an age-related process. The premature loss of TREC+ cells reflects an excessive proliferative stress relative to thymic output in vivo⁵¹. The difference in basal TREC expression, while statistically significant (p=0.0346), was minimized in the older groups due to age-related loss in TREC+ cells (FIG. 4C). In younger groups, there was an obvious difference in TREC expression in the T-cell population of MDS patients compared to control (p=0.0455).

The defects in telomerase function may reflect changes in population dynamics or a reduced capacity for telomere repair that causes premature telomere attrition and increased immune aging. Naïve, but not memory cells rely on telomerase function to maintain survival and proliferative capacity³³. To determine if the defective telomere repair affects naïve T-cells or memory cells directly, TCR triggered telomerase activity was measured in sorted populations of these cells based on their phenotype. TREC+ cells were confirmed to be present only within naïve (CD45RA⁺CD45RO⁻) and not the memory (CD45RO⁺CD45RA⁻) T-cells. Telomere length was then compared in sorted naïve and memory T-cells from a subset of the age-matched MDS patients (n=8) and healthy controls (n=8) (FIGS. 4D and 4E). The mean relative telomere length was dramatically longer in naïve (1.527) T-cells compared to memory cells (0.85) in controls (1.527) (p=0.006). In MDS patients by contrast, the telomere length in naïve cells was similar to that of control and patient memory cells and differed significantly with naive cells from controls (0.5, p=0.0175). While there was a slight difference in the memory population, the case-control difference was not statistically significant (p=0.383). Thus, inappropriate telomere erosion primarily affected naïve T-cells in MDS patients (FIG. 4F). Telomerase activity in naïve T-cells from MDS patients (22.76, 95%, 17.33-26.94) compared to healthy controls (35.57, 95%, 24.07-55.70) (p=0.0207) was also significantly reduced and resembled the activity of normal memory cells (FIG. 4F), while the memory T-cells population showed no difference (p=0.5054) (FIG. 4F). These results indicate that the primary telomerase insufficiency of T-cells resides with a defect within the naïve T-cell compartment so it is therefore independent of prior antigen exposure.

### Example 6

### Proliferative Defects in MDS T-Cells Correlates with Telomerase Deficiency

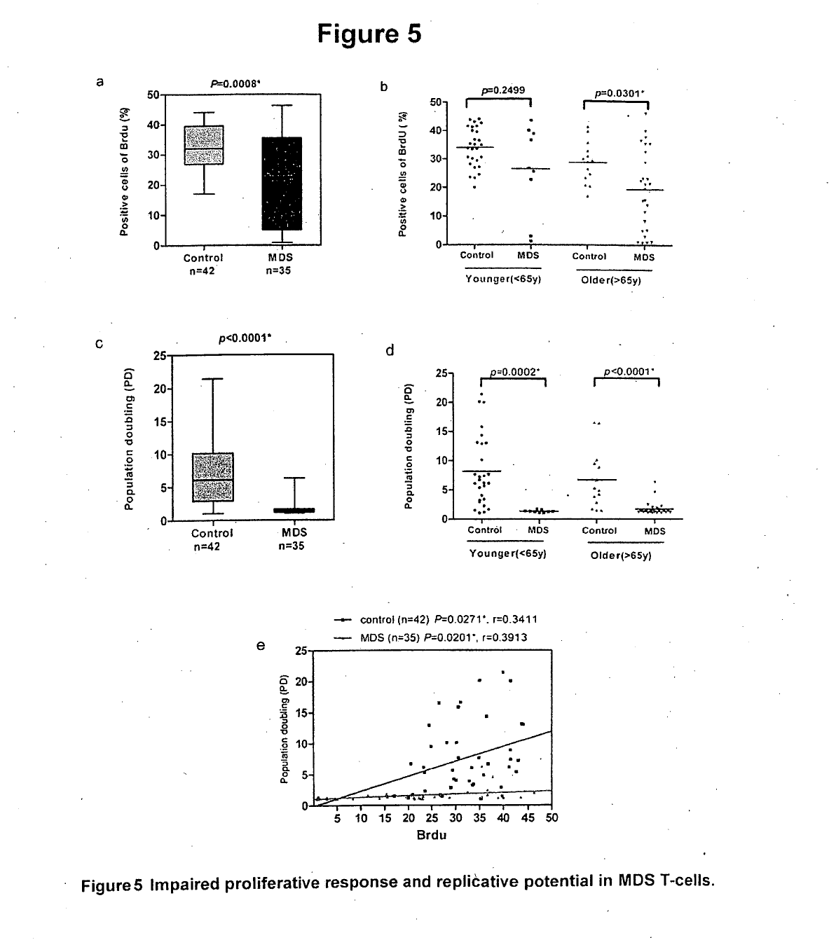

The ability of T-cells to induce telomerase activity is closely linked to the replicative burst potential that is necessary to mount an immune response³³. To define the proliferative capacity of T-cells, the inventors measured both the percentage of cells capable of proliferation (i.e., percentage of BrdU positive cells) and the number of rounds of division within the 3-day stimulation period. First, the percentage of BrdU positive T-cells was compared among MDS patients and healthy controls and found to be significantly reduced in MDS patients (median=22.30, 95% CI, 16.26-26.25) compared to healthy donors (median=32.00, 95% CI, 29.96-34.56) (p=0.0008) (FIG. 5A) indicating that TCR-induced proliferation is impaired. This difference was statistically different in older MDS patients (p=0.0301), but not statistically different in the younger group suggesting that younger age and relatively longer telomere length may be sufficient for cells to transition from G0/1 to S-phase (FIG. 5B), although two younger patients appear to be particularly defective. Next, the replicative burst potential was examined. Regardless of the starting point in TREC expression, linear dilution occurs in vitro as a measure of population doubling (PD) response. After in vitro stimulation, the TREC copy number decreased from a mean of 80 to 15 copies per cell in controls indicating that an average of 6 PDs occurred in healthy T-cells. In MDS, the TREC copy number decreased from a mean of 32 to 25 copies per cell indicating that 1.3 PDs occurred within the 3-day stimulation period (FIG. 5C), which was significantly lower than controls (p<0.0001). For the younger and older groups, there was a statistical case-control difference (FIG. 5D) in PDL indicating that all patients have a severely defective replication burst potential. PD in older and younger controls were similar although the basal TREC level was significantly lower (FIG. 4C) suggesting that the proliferative burst potential is not compromised. Comparing PD level to BrdU showed that S-phase transition does not ensure that normal proliferative burst is intact in MDS (FIG. 5E). Collectively, these results indicate that MDS T-cells have been exposed to more proliferative stress in vivo and that they have prematurely lost cells within the naïve compartment driven by a defect in telomere and telomerase activity within the naïve compartment. These results point to a primary telomere repair defect associated with accelerated turnover, premature aging and impaired homeostasis within the T-cell compartment.

### Example 7

### No Correlation Between Clinical Classification and Telomere Repair

The telomerase and hTERT deficiency in MDS T-cells occurred for all patients fell below the 95% confidence interval for controls. Measurements including telomere length, hTERT, TREC levels and telomerase activity were correlated to IPSS score, WHO subtype and cytogenetics in the MDS cases in Table 2. Except for CD69, which showed a decrease in higher-risk MDS patients, there was no statistically significant association with disease stratification, including risk factor for AML progression, suggesting that this abnormality may be consistent across all subtypes.

TABLE 2
Clinical characteristics and telomerase measurements in peripheral blood CD3+T cells of 35
myelodysplastic syndrome (MDS) cases

| | | | RTL | | hTERT | | TA | | CD69% | |
|---|---|---|---|---|---|---|---|---|---|---|
| | | | | p- | | p- | TA | p- | | p- |
| [symbol] tic | n | % | mean (SD) | value | mean (SD) | value | mean (SD) | value | mean (SD) | value |
| [symbol] mediate-1 | 21 | 60 | 1.10 (0.46) | 0.11 | 18.84 (6.68) | 0.1 | 16.54 (5.92) | 0.09 | 72.74 (13.39) | 0.02* |
| [symbol] rte-2 + high | 14 | 40 | 1.39 (0.46) | | 15.12 (6.77) | | 13.45 (4.29) | | 56.28 (20.20) | |
| [symbol] cs | | | | | | | | | | |
| | 15 | 43 | 1.06 (0.35) | 0.09 | 16.53 (7.43) | 0.38 | 14.48 (5.11) | 0.68 | 65.21 (19.70) | 0.96 |
| | 20 | 57 | 1.35 (0.53) | | 17.79 (6.57) | | 15.77 (5.73) | | 66.05 (17.78) | |
| S subtype | | | | | | | | | | |
| [symbol] ARS | 6 | 17 | 1.45 (0.56) | 0.4 | 21.17 (11.78) | 0.58 | 15.09 (7.18) | 0.79 | 69.43 (16.22) | 0.35 |
| [symbol] CMD-RS | 15 | 43 | 1.11 (0.36) | | 16.36 (5.14) | | 15.57 (5.53) | | 69.55 (17.96) | |
| [symbol] AEB2 | 14 | 40 | 1.25 (1.55) | | 16.52 (5.76) | | 14.90 (4.92) | | 59.94 (19.29) | |

| | BrdU % | | TREC | | PDL | |
|---|---|---|---|---|---|---|
| [symbol] tic | mean (SD) | p-value | mean (SD) | p-value | mean (SD) | p-value |
| [symbol] mediate-1 | 24.83 (15.00) | 0.14 | 38.27 (21.77) | 0.8 | 1.77 (1.33) | 0.56 |
| [symbol] rte-2 + high | 16.48 (12.85) | | 52.21 (70.80) | | 1.39 (0.38) | |
| [symbol] cs | | | | | | |
| | 20.55 (15.82) | 0.88 | 43.56 (25.43) | 0.28 | 1.82 (1.52) | 0.61 |
| | 21.78 (13.89) | | 44.76 (61.47) | | 1.45 (0.39) | |
| S subtype | | | | | | |
| [symbol] ARS | 23.83 (16.28) | 0.8 | 81.84 (105.7) | 0.66 | 1.77 (1.39) | 0.86 |
| [symbol] CMD-RS | 20.24 (15.14) | | 36.15 (21.41) | | 1.68 (1.31) | |
| [symbol] AEB2 | 21.23 (14.13) | | 36.81 (25.37) | | 1.47 (0.41) | |

RTL = Relative telomere length;
TA = Telomerase activity;
hTERT = Human telomerase reverse transcriptase
IPSS = International Prognostic Scoring System;
WHO = World Health Organization [symbol]
indicates data missing or illegible when filed

This study was conducted to determine the mechanism for altered T-cell homeostasis in MDS and to determine if telomerase insufficiency explains the similarities between T-cell abnormalities in MDS and AA. T-cells are one of only a few somatic cells that retain telomerase function where it is critical to control naïve T-cell survival, homeostatic regulation, and antigenic diversity³⁶. TCR stimulation causes the induction of telomerase enzymatic activity by increasing hTERT transcription³³. Telomerase enzyme activity and hTERT transcription were significantly impaired in MDS T-cells with all patients falling below the 95% CI compared to controls (FIGS. 1A-1C and 2A-2D). Enzyme activity is normally required in naïve (CD45RA+/CD45R−) T-cells more so than in memory cells, which was previously shown to correlate with the replication potential and survival of these cells after stimulation³⁶. Studies of telomere biology in a number of human somatic cell lineages have shown that aging leads to shortened telomere length in vivo and with cell division in vitro^{33,42}. The defect in telomere function in MDS T-cells was independent of age, but age may have played an important role in initiating the defect. Because of defective thymic efflux, naïve T-cells become more reliant on homeostatic proliferation in order to maintain normal compartmentalization in the periphery after 50 or 60 years old¹⁹. It is possible that cytokines and self-antigens, greater homeostatic proliferation by IL-2 receptor common (IL-2Rc) cytokines, and stimulation by self-antigens under homeostatic mechanisms prompted the initiation of an accelerated age-related process that was not evident clinically until older age in these patients^{45,50-52}. Collectively, these results possibly explain the strong relationship between immune defects, age, and immune-mediated impairments in hematopoiesis in MDS that occurs around age 50 to 60 years old.

Interestingly, the significant case-control difference in telomerase function resided within the naïve population (FIG. 4E), and memory cells displayed no difference. These results suggest that the mechanism regulating survival and proliferative capacity of naïve and memory T-cells differ and explains why memory T-cells become preferentially expanded in MDS and in AA. To exclude the possibility that the difference in telomerase function was due to general defects in TCR early signaling events caused by the memory expansion and repertoire contraction, the inventors measured CD69 and found similar expression in cases and controls indicating that the telomerase insufficiency represents a specific cellular defect. Therefore, inherent defects in the proliferation of naïve T-cells are likely to favor expansion of memory cells limiting the diversity within the peripheral T-cell compartment and inducing contraction of the T-cell repertoire.

Many experiments have revealed severe repertoire contraction in MDS and AA T-cells that apparently stem from this inherent defect in maintaining the naïve T-cell population^{6,7,54}. In this study, the inventors show that the telomerase defect was associated with loss of the naïve cells, as shown by a decrease in TREC+ cells (FIGS. 4A-4G). The inventors measured TREC expression and also measured the replicative potential of the T-cell compartment by determining TREC dilution (i.e., PD) after stimulation. PD was compromised in both younger and older MDS patients. Although some cells, primarily those of younger age, retained the ability to undergo S-phase transition and incorporate BrdU after stimulation, population doubling was severely impaired (FIGS. 5A-5E). In MDS, telomere shortening in myeloid cells has been reported previously, but has been attributed to proliferation-induced stress caused by inflammation and aging^{25,29,30}. Mutations in hTERT failed to be detected in the inventors' recent study³⁰ and telomere length is increased in myeloid cells after transformation to high-risk MDS and AML³¹ suggesting that telomere components are unlikely to be mutated within the stem cell compartment. While telomerase function is absent, in most normal human somatic cells, it is required for survival and homeostatic proliferation of naïve T-cells³²⁻³⁵. As naïve T-cells differentiate into memory cells, after antigen stimulation, telomerase activity progressively declines³⁶ and cell survival as well as proliferation is maintained through a distinct mechanism. If telomere repair is insufficient, T-cells would be more severely affected than hematopoietic progenitors and stem cells since each antigen encounter results in a 1000-fold clonal expansion³⁷. Since intact telomerase activity is necessary for the survival of naïve T-cells and peripheral homeostasis and population doubling (PD) occurs every 4 months within the naïve T-cell compartment in healthy individual, it is essential that telomerase function is intact to maintain population diversity and to prevent autoimmunity³⁷⁻³⁹.

In total, these results show that the T-cell compartment is under physiological stress due to inherent deficiency in telomerase that leads to accelerated turnover and premature senescence-associated proliferation arrest, loss of naïve T-cells that consequently leads to the skewed repertoire diversity that is well-described in MDS and in AA^{67,20,54-57}. Overexpression of hTERT improved apoptotic resistance of naïve T-cells from patients with rheumatoid arthritis with similar defects in inducible hTERT expression^{32,34,45}. Diseases of telomerase deficiency include congenital and acquired aplastic anemia, dyskeratosis congenita (DC), pulmonary fibrosis and hepatic nodular regenerative hyperplasia and cirrhosis display abnormities in telomere repair in their hematopoietic compartment leading to bone marrow failure and immune deregulations. Multiple congenital or acquired mutations have been detected in hematopoietic progenitors including hTERT, hTERC, or components of the shelterin complex; all key components of the telomerase complex^{24-26,58}. Described herein is a telomere repair defect in MDS that is due to hTERT insufficiency in naïve T-cells. The data indicates that this abnormality is an intrinsic feature of MDS T-cells since it occurs universally in patients and is independent of IPSS or WHO classification, but it is unlikely to arise from a mutation in hTERT. Hematopoietic improvement and reconstitution of T-cell diversity suggests that these defects are correctable after immunosuppressive therapy^{9,15}. The inventors' previous data suggested that eATG dramatically halts proliferation of naïve T-cells in patients with hematologic improvement, providing more evidence that the telomere repair defect is reversible The control of hTERT transcription is normally regulated by several transcription factors including c-Myc, WT1, and p53 that may be uncoupled in MDS^{33,35}.

In hematopoietic stem cells, critically short telomeres are recognized as DNA damage and recruit proteins involved in the DNA-damage response pathway, which stimulates apoptosis. Given that memory T-cells escape this regulatory pathway, memory T-cells would have a proliferative advantage in this setting and would be more likely to accumulate. In immunosuppressive therapy responsive MDS patients, hematopoiesis may be suppressed by the entry of autoreactive memory T-cells into the bone marrow microenvironment where they recognize diverse self-antigens and directly eliminate hematopoietic progenitors or produce suppressive cytokines that result in dysregulated myelopoiesis. If myeloid clones gain mutations or a survival advantage then immune deregultions may no longer drive disease pathogenesis although it may be present as an underlying abnormality.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES I

1 Rollison, D. E. et al. Epidemiology of myelodysplastic syndromes and chronic myeloproliferative disorders in the United States, 2001-2004, using data from the NAACCR and SEER programs. Blood 112, 45-52, doi:blood-2008-01-134858 [pii] 10.1182/blood-2008-01-134858 (2008).
2 Ma, X., Does, M, Raza, A. & Mayne, S. T. Myelodysplastic syndromes: incidence and survival in the United States. Cancer 109, 1536-1542, doi:10.1002/cncr.22570 (2007).
3 Sanz, G. F., Sanz, M. A. & Greenberg, P. L. Prognostic factors and scoring systems in myelodysplastic syndromes. Haematologica 83, 358-368 (1998).
4 Raza, A. et al. Excessive proliferation matched by excessive apoptosis in myelodysplastic syndromes: the cause-effect relationship. Leuk Lymphoma 27, 111-118, doi:10.3109/10428199709068277 (1997).
5 Epling-Burnette, P. K. & List, A. F. Advancements in the molecular pathogenesis of myelodysplastic syndrome. Curr Opin Hematol 16, 70-76, doi: 10.1097/MOH.0b013e3283257ac700062752-200903000-00004 [pii] (2009).
6 Kook, H. et al. Increased cytotoxic T cells with effector phenotype in aplastic anemia and myelodysplasia. Exp Hematol 29, 1270-1277, doi:S0301-472X(01)00736-6 [pii] (2001).
7 Maciejewski, J. P., Hibbs, J. R., Anderson, S., Katevas, P. & Young, N. S. Bone marrow and peripheral blood lymphocyte phenotype in patients with bone marrow failure. Exp Hematol 22, 1102-1110 (1994).
8 Sloand, E. M. et al. T-cell immune responses to Wilms tumor 1 protein in myelodysplasia responsive to immunosuppressive therapy. Blood 117, 2691-2699, doi:blood-2010-04-277921 [pii] 10.1182/blood-2010-04-277921 (2011).
9 Zou, J. X. et al. Altered naïve and memory CD4+ T-cell homeostasis and immunosenescence characterize younger patients with myelodysplastic syndrome. Leukemia 23, 1288-1296, doi:leu200914 [pii] 10.1038/leu.2009.14 (2009).
10 Campioni, D. et al. Evidence for a role of TNF-related apoptosis-inducing ligand (TRAIL) in the anemia of myelodysplastic syndromes. Am J Pathol 166, 557-563, doi:S0002-9440(10)62277-8 [pii] 10.1016/S0002-9440(10)62277-8 (2005).
11 Biesma, D. H., van den Tweel, J. G. & Verdonck, L. F. Immunosuppressive therapy for hypoplastic myelodysplastic syndrome. Cancer 79, 1548-1551, doi:10.1002/(SICI)1097-0142(19970415)79:8<1 548::AID-CNCR16>3.0.CO; 2-Y [pii] (1997).
12 Molldrem, J. J. et al. Antithymocyte globulin for patients with myelodysplastic syndrome. Br J Haematol 99, 699-705 (1997).
13 Deeg, H. J. et al. Hematologic responses of patients with MDS to antithymocyte globulin plus etanercept correlate with improved flow scores of marrow cells. Leuk Res 28, 1177-1180, doi:10.1016/j.leukres.2004.03.016S0145212604001225 [pii] (2004).
14 Killick, S. B. et al. A pilot study of antithymocyte globulin (ATG) in the treatment of patients with ‘low-risk’ myelodysplasia. Br J Haematol 120, 679-684, doi:4136 [pii] (2003).
15 Molldrem, J. J. et al. Haematological response of patients with myelodysplastic syndrome to antithymocyte globulin is associated with a loss of lymphocyte-mediated inhibition of CFU-GM and alterations in T-cell receptor Vbeta profiles. Br J Haematol 102, 13.14-1322 (1998).
16 Passweg, J. R. et al. Immunosuppressive therapy for patients with myelodysplastic syndrome: a prospective randomized multicenter phase III trial comparing antithymocyte globulin plus cyclosporine with best supportive care—SAKK 33/99. J Clin Oncol 29, 303-309, doi:JCO.2010.31.2686 [pii] 10.1200/JCO.2010.31.2686 (2011).
17 Scott, B. L. et al. Anti-thymocyte globulin plus etanercept as therapy for myelodysplastic syndromes (MDS): a phase II study. Br J Haematol 149, 706-710, doi:BJH8145 [pii] 10.1111/j.1365-2141.2010.08145.x (2010).
18 Yazji, S. et al. Antithymocyte globulin (ATG)-based therapy in patients with myelodysplastic syndromes. Leukemia 17, 2101-2106, doi:10.1038/sj.leu.2403124 2403124 [pii] (2003).
19 Sloand, E. M., Wu, C. O., Greenberg, P., Young, N. & Barrett, J. Factors affecting response and survival in patients with myelodysplasia treated with immunosuppressive therapy. J Clin Oncol 26, 2505-2511, doi:JCO.2007.11.9214 [pii] 10.1200/JCO.2007.11.9214 (2008).
20 Saunthararajah, Y. et al. HLA-DR15 (DR2) is overrepresented in myelodysplastic syndrome and aplastic anemia and predicts a response to immunosuppression in myelodysplastic syndrome. Blood 100, 1570-1574 (2002).
21 Saunthararajah, Y., Nakamura, R., Wesley, R., Wang, Q. J. & Barrett, A. J. A simple method to predict response to immunosuppressive therapy in patients, with myelodysplastic syndrome. Blood 102, 3025-3027, doi:10.1182/blood-2002-11-3325 2002-11-3325 [pii] (2003).
22 Stadler, M. et al. A prospective, randomised, phase II study of horse antithymocyte globulin vs rabbit antithymocyte globulin as immune-modulating therapy in patients with low-risk myelodysplastic syndromes. Leukemia 18, 460-465, doi:10.1038/sj.leu.2403239 2403239 [pii] (2004).
23 Young, N. S. Pathophysiologic mechanisms in acquired aplastic anemia. Hematology Am Soc Hematol Educ Program, 72-77, doi:2006/1/72 [pii] 10.1182/asheducation-2006.1.72 (2006).
24 Fibbe, W. E. Telomerase mutations in aplastic anemia. N Engl J Med 352, 1481-1483, doi:352/14/1481 [pii] 10.1056/NEJMe058015 (2005).
25 Calado, R. T. & Young, N. S. Telomere maintenance and human bone marrow failure. Blood 111, 4446-4455, doi:blood-2007-08-019729 [pii] 10.1182/blood-2007-08-019729 (2008).
26 Bessler, M., Wilson, D. B. & Mason, P. J. Dyskeratosis congenita and telomerase. Curr Opin Pediatr 16, 23-28, doi:00008480-200402000-00006 [pii] (2004).
27 Sasa, G., Ribes-Zamora, A., Nelson, N. & Bertuch, A. Three novel truncating TINF2 mutations causing severe dyskeratosis congenita in early childhood. Clin Genet, doi:10.1111/j.1399-0004.2011.01658.x (2011).
28 Theimer, C. A., Finger, L. D., Trantirek, L. & Feigon, J. Mutations linked to dyskeratosis congenita cause changes in the structural equilibrium in telomerase RNA. Proc Natl Acad Sci USA 100, 449-454, doi: 10.1073/pnas.242720799242720799 [pii] (2003).
29 Cawthon, R. M., Smith, K. R., O'Brien, E., Sivatchenko, A. & Kerber, R. A. Association between telomere length in blood and mortality in people aged 60 years or older. Lancet 361, 393-395, doi:S0140-6736(03)12384-7 [pii] 10.1016/S0140-6736(03)12384-7 (2003).
30 Rollison, D. E. et al. Telomere length in myelodysplastic syndromes. Leuk Lymphoma, doi: 10.3109/10428194.2011.568648 (2011).
31 Boultwood, J. et al. Telomere length in myelodysplastic syndromes. Am J Hematol 56, 266-271, doi: 10.1002/(SICI) 1096-8652(199712)56:4<266::AID-AJH12>3.0.CO; 2-7 [pii] (1997).
32 Koetz, K. et al. T cell homeostasis in patients with rheumatoid arthritis. Proc Natl Acad Sci USA 97, 9203-9208, doi:97/16/9203 [pii] (2000).
33 Roth, A. et al. Telomerase levels control the lifespan of human T lymphocytes. Blood 102, 849-857, doi: 10.1182/blood-2002-07-20152002-07-2015 [pii] (2003).
34 Schonland, S. O. et al. Premature telomeric loss in rheumatoid arthritis is genetically determined and involves both myeloid and lymphoid cell lineages. Proc Natl Acad Sci USA 100, 13471-13476, doi:10.1073/pnas.22335611002233561100 [pii] (2003).
35 Weng, N. P., Levine, B. L., June, C. H. & Hodes, R. J. Regulated expression of telomerase activity in human T lymphocyte development and activation. J Exp Med 183, 2471-2479 (1996).
36 Fritsch, R. D. et al. Stepwise differentiation of CD4 memory T cells defined by expression of CCR7 and CD27. J Immunol 175, 6489-6497, doi:175/10/6489 [pii] (2005).
37 Macallan, D.C. et al. Measurement and modeling of human T cell kinetics. Eur J Immunol 33, 2316-2326, doi: 10.1002/eji.200323 763 (2003).
38 Freitas, A. A. & Rocha, B. Population biology of lymphocytes: the flight for survival. Annu Rev Immunol 18, 83-111, doi:18/1/83 [pii] 10.1146/annurev.immunol.18.1.83 (2000).
39 Freitas, A. A. & Rocha, B. Peripheral T cell survival. Curr Opin Immunol 11, 152-156, doi:S0952791599800260 [pii] (1999).
40 Harris, N. L. et al. World Health Organization classification of neoplastic diseases of the hematopoietic and lymphoid tissues: report of the Clinical Advisory Committee meeting-Airlie House, Va., November 1997. J Clin Oncol 17, 3835-3849 (1999).
41 Vardiman, J. W., Harris, N. L. & Brunning, R. D. The World Health Organization (WHO) classification of the myeloid neoplasms. Blood 100, 2292-2302, doi: 10.1182/blood-2002-04-1199 (2002).
42 Shen, J. et al. Short telomere length and breast cancer risk: a study in sister sets. Cancer Res 67, 5538-5544, doi:67/11/5538.[pii] 10.1158/0008-5472.CAN-06-3490 (2007).
43 McGrath, M., Wong, J. Y., Michaud, D., Hunter, D. J. & De Vivo, I. Telomere length, cigarette smoking, and bladder cancer risk in men and women. Cancer Epidemiol Biomarkers Prev 16, 815-819, doi:16/4/815 [pii] 10.1158/1055-9965.EP1-06-0961 (2007).
44 Pongers-Willemse, M. J. et al. Real-time quantitative PCR for the detection of minimal residual disease in acute lymphoblastic leukemia using junctional region specific TaqMan probes. Leukemia 12, 2006-2014 (1998).
45 Fujii, H., Shao, L., Colmegna, I., Goronzy, J. J. & Weyand, C. M. Telomerase insufficiency in rheumatoid arthritis. Proc Natl Acad Sci USA 106, 4360-4365, doi:0811332106 [pii] 10.1073/pnas.0811332106 (2009).
46 Tateno, M., Honda, M., Kawamura, T., Honda, H. & Kaneko, S. Expression profiling of peripheral-blood mononuclear cells from patients with chronic hepatitis C undergoing interferon therapy. J Infect Dis 195, 255-267, doi:JID36902 [pii] 10.1086/509893 (2007).
47 Broers, A. E. et al. Quantification of newly developed T cells in mice by real-time quantitative PCR of T-cell receptor rearrangement excision circles. Exp Hematol 30, 745-750, doi:S0301472X02008251 [pii] (2002).
48 Chen, X. et al. Prediction of T-cell reconstitution by assessment of T-cell receptor excision circle before allogeneic hematopoietic stem cell transplantation in pediatric patients. Blood 105, 886-893, doi:10.1182/blood-2004-04-14052004-04-1405 [pii] (2005).
49 Svenson, U., Ljungberg, B. & Roos, G. Telomere length in peripheral blood predicts survival in clear cell renal cell carcinoma. Cancer Res 69, 2896-2901, doi:0008-5472.CAN-08-3513 [pii] 10.1158/0008-5472.CAN-08-3513 (2009).
50 Poulin, J. F. et al. Direct evidence for thymic function in adult humans. J Exp Med 190, 479-486 (1999).
51 Douek, D. C. et al. Changes in thymic function with age and during the treatment of HIV infection. Nature 396, 690-695, doi:10.1038/25374 (1998).
52 Kong, F., Chen, C. H. & Cooper, M. D. Thymic function can be accurately monitored by the level of recent T cell emigrants in the circulation. Immunity 8, 97-104, doi:S 1074-7613(00)80462-8 [pii] (1998).
53 Hodes, R. J., Hathcock, K. S. & Weng, N. P. Telomeres in T and B cells. Nat Rev Immunol 2, 699-706, doi:10.1038/nri890nri890 [pii] (2002).
54 Wlodarski, M. W. et al. Molecular strategies for detection and quantitation of clonal cytotoxic T-cell responses in aplastic anemia and myelodysplastic syndrome. Blood 108, 2632-2641, doi:2005-09-3902 [pii] 10.1 182/blood-2005-09-3902 (2006).
55 Feng, X. et al. Cytokine signature profiles in acquired aplastic anemia and myelodysplastic syndromes. Haematologica 96, 602-606, doi:haematol.2010.030536 [pii] 10.3324/haematol.2010.030536 (2011).
56 Plasilova, M., Risitano, A. & Maciejewski, J. P. Application of the molecular analysis of the T-cell receptor repertoire in the study of immune-mediated hematologic diseases. Hematology 8, 173-181, doi:10.1080/1024533031000107505LU6GQ38XG20M 11P6T [pii] (2003).
57 Sloand, E. M. et al. Fas-mediated apoptosis is important in regulating cell replication and death in trisomy 8 hematopoietic cells but not in cells with other cytogenetic abnormalities. Blood 100, 4427-4432, doi:10.1182/blood-2002-01-00962002-01-0096 [pii] (2002).
58 Calado, R. T. Telomeres and marrow failure. Hematology Am Soc Hematol Educ Program, 338-343, doi:2009/1/338 [pii] 10.1182/asheducation-2009.1.338 (2009).

### Materials and Methods for Examples 8-16

Cell Isolation in Patients and Healthy Controls.

MDS patients were recruited from 2005-2010 through a collaborative study involving four centers: the Hematologic Malignancy Program at the Moffitt Cancer Center (“Moffitt”), the Cleveland Clinic Cancer Center, UCLA Medical Center and Penn State Cancer Center. All study participants signed informed consents approved by the institutional review boards (IRB) at the participating institutions. All diagnoses were histologically-confirmed after review at the participating center and classified according to standard criteria including the World Health Organization (WHO)(14) and International Prognostic Scoring System (IPSS)(1). Inclusion into this study (n=35) was based on having a sufficient volume of frozen peripheral blood mononuclear (PBMCs) available for T-cell isolation (30×10⁶ cells). Details describing the isolation of PBMCs and purification of CD3+ T-cells is provided herein.

Measurement of Telomere Length.

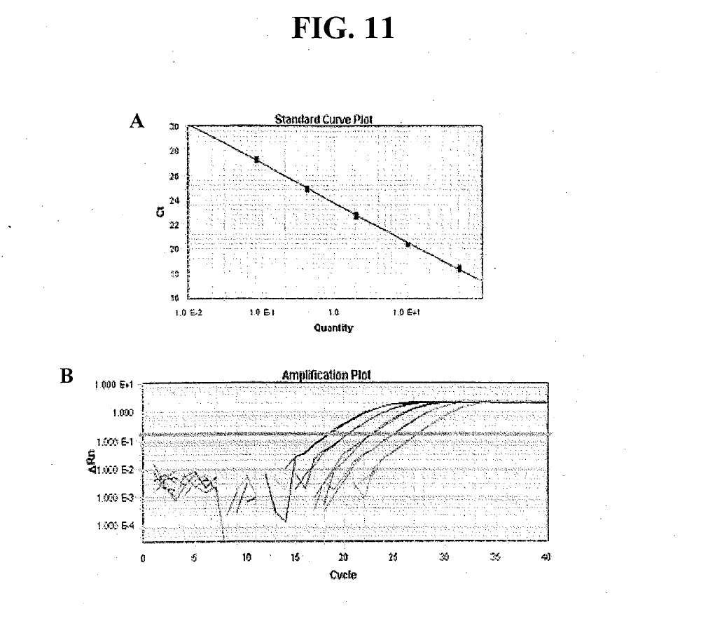

DNA was extracted from unstimulated purified CD3+ T-cells using the PureLink Genomic DNA Kits according to manufacturer's instructions (Invitrogen, Carlsbad, Calif.), and relative telomere lengths were measured by a modified version of the quantitative real-time (qRT) PCR-based telomere assay, as described previously(15, 16). Details of the assay are provided herein and in FIGS. 11A-11B.

T-Cell Activation.

Purified CD3+ T-cells were stimulated in RPMI 1640 medium (Gibco, Grand Island, N.Y.) containing 10% FBS with CD3/CD28 T-cell activator beads (Dynabead®, Invitrogen, Carlsbad, Calif., USA) for 3 days.

Measurement of TREC DNA in Purified T-Cells.

Signal-joint T-cell receptor excision circles (sjTRECs) are circular extrachromosomal fragments of DNA generated during TCR gene rearrangement in the thymus(17). Since they are non-transferable to daughter cells, expression decreases with each round of cell division in culture and quantification is a reliable estimate of population doubling(18). Genomic DNA was extracted from CD3+ T-cell, as described above. Detailed methods for sjTREC quantification can be found herein.

Measurement of hTERT mRNA expression. Details of qRT-PCR methods is provided in the herein.

Telomerase Activity.

Telomerase activity was determined using the telomerase PCR-ELISA kit (TRAP) (Roche Molecular Biochemicals, Indianapolis, Ind., USA) according to the manufacturer's instructions. Further details are provided herein. Experiments were carried out at least twice to improve the reliability of the results. The relative telomerase activity within each sample was calculated as follows: (the absorbance of the sample—the absorbance of the heat-treated sample)/the absorbance of the internal standard of the sample.

Flow Cytometry to Detect BrdU Incorporation.

To access entry into S-phase, bromodeoxyuridine (BrdU) incorporation (BrdU flow kit, BD Biosciences, San Diego, Calif.) was used, as described previously(19). Further details are provided herein.

NaïVe and Memory Cell Purification and Flow Assays.

T-cells subsets were isolated from purified CD3+ T-cells using CD45RO and CD45RA antibody staining and isolation with the Aria flow sorter (BD Biosciences). Subset purity was >95% in post-sort analyses. Memory (CD45RO+CD45RA−) and naïve (CD45RA+CD45RO−)(17) cells were stimulated with CD3/CD28 activator beads (Dynabeads®, Dynal Corp.) for 3 days. Telomere length was detected in sorted naïve and memory T-cells before stimulation and telomerase activity and hTERT expression was measured using the TRAP assay and qRT-PCR reaction, respectively, before and after the 3 days of stimulation. DAPI exclusion discriminated viable and dead cells during activation and CD69 staining was used to determine the TCR response. Detailed methods are provided herein.

Sequencing of hTERT Promoter.

The core promoter of hTERT was amplified using genomic DNA from purified CD3+ T-cells for sequence analysis. Detailed methods are provided herein.

Statistical Analysis.

Telomere length, proliferation (BrdU and PD), and hTERT mRNA data were compared as continuous variables between MDS cases and controls using the Wilcoxon rank-sum test. Data on telomerase activity was normally distributed and compared as a continuous variable using a T-test. The mean age of controls was 52 (range 17-84 y) and MDS cases was 67 (range 28-87 y) (Wilcoxon, p=0.1). For all variables, the statistical significance of the interaction with age as a continuous variable and sex in relation to case-control status was tested by including an interaction term in the logistic regression model of log-transformed data. However, hTERT mRNA data was square-root transformed to achieve normality because log-transformation failed to achieve a normal distribution. Correlations between telomere length and age and hTERT activity and mRNA expression were assessed using the Spearman con-elation coefficient.

Since T-cell homeostasis is altered by thymic involution, and younger MDS patients (i.e. under the age of 65 years old) are suspected of having a different pathobiologic mechanism for impaired hematopoiesis related to an immunological attack, the analyses was stratified by age <65 or ≧65 years. For these subset analyses, the 5 youngest healthy controls were omitted to better approximate the age range among younger MDS cases. In these subgroups, the mean age of controls was 47 y (range 26-63 y, n=23) and the mean age of cases was 52 y (n=9, range 28-61 y), (Wilcoxon, p=0.27). The older group was balanced with regard to age with a mean of 73 (n=14, range 65-84 y) in controls and a mean of 73 (n=26, range 65-87 y) in cases (Wilcoxon, p=0.97). All statistical analyses were performed with the SPSS 19.0 software package (SPSS Inc., Chicago, Ill., USA).

Supplemental Methods for Isolation of Peripheral Blood Mononuclear Cells and T-Cells Purification.

PBMCs were isolated by Ficoll-Hypaque (Amersham Pharmacia Biothech, Piscataway, N.J., USA) centrifugation. For healthy controls (n=42), buffy coats were collected from donations at the Southwest Florida Blood Services in St Petersburg, Fla. along with information on age and sex. PBMCs were isolated by Ficoll-Hypaque centrifugation, frozen, and stored in liquid nitrogen. Purified CD3+ T-cells from patients and controls were isolated from PBMC using negative selection (Stemcell technologies, Vancouver, BC, Canada) immediately prior to functional experiments. Briefly, the enrichment cocktail was added to PBMCs at 50 μl/ml and incubated 20 min at room temperature and centrifugation was used to separate the CD3+ T-cell population.

Supplemental Methods for hTERT mRNA Analysis by qRT-PCR.

Total RNA was extracted from purified T-cells using the RNeasy Mini Kit (Qiagen, Chatsworth, Calif.). Reverse transcription was performed using the high-capacity cDNA reverse transcription kit (Applied Biosystems) in accordance with the manufacturer's suggestions. Relative hTERT gene expression was measured by a qRT-PCR with 18S rRNA used as a reference gene. Expression levels of hTERT mRNA and 18S rRNA were evaluated with Taqman Probes obtained from ABI. The reaction mixture consisted of 1× Taqman mixture, 1× primer of hTERT or 18S rRNA. All samples for both the hTERT and 18S rRNA were tested in triplicate in the same plate. The expression level of hTERT for each sample was calculated with the ΔΔCt method(1).

Supplemental Information for Telomere Length Measurements.

This method has been used in several previous case-control studies of telomere length and cancer(2). Briefly, the telomere repeat copy number to single gene copy number (T/S) ratio was determined using an Applied Biosystems (Foster City, Calif.) 7900 HT PCR system in a 96-well format. Ten ng of genomic DNA was used for either the telomere or hemoglobin (hgb) PCR reaction and the primers used were as follows: Tel-1b primer (CGG TTT GTT TGG GTT TGG GTT TGG GTT TGG GTT TGG GTT) (SEQ ID NO:1), Tel-2b primer (GGC TTG CCT TAC CCT TAC CCT TAC CCT TAC CCT TAC CCT) (SEQ ID NO:2), hgb1 primer (GCTTCTGACACAACTGTGTTCACTAGC) (SEQ ID NO:3), and hgb2 primer (CACCAACTTCATCCACGTTCACC) (SEQ ID NO:4). All samples were analyzed by both the telomere and hemoglobin reactions, and the analysis was performed in triplicate on the same plate. In addition to the test samples, each 96-well plate contained a five-point standard curve from 0.08 ng to 50 ng using genomic DNA. The standard curve was run in each plate to assess inter-assay variations in PCR efficiency and a sample of this data is shown in FIG. 11B. The T/S ratio (-dCt) for each sample was calculated by subtracting the median hemoglobin threshold cycle (Ct) value from the median telomere Ct value. The relative T/S ratio (-ddCt) was determined by subtracting the T/S ratio of the 10.0 ng standard curve point from the T/S ratio of each unknown sample(1, 3).

Telomerase Activity Assay (TRAP).

An equal amount of lysis extract from each sample (2×10⁵ cells) was incubated with a biotinylated telomerase substrate oligonucleotide (P1-TS primer) at 25° C. for 30 minutes. At the same time, a heat-treated (85° C. for 10 minutes) negative control was included for each sample during incubation. Next, the extended products were amplified using PCR with P1-TS primers. The PCR program consisted of 30 cycles at 95° C. for 30 sec, 50° C. for 30 sec, and 72° C. for 90 sec, followed by 72° C. for 10 min. After 10 minutes of denaturation, the PCR-amplified products for each sample were hybridized with buffer T and buffer IS for 2 hours, respectively, and immobilized onto a streptavidin-coated microplate. Negative controls were only treated with hybridization T buffer. The resulting products are then detected with the anti-DIG-HRP and detected with TMB substrate solution. The absorbance of the final colored reaction was measured at 450 nm with a reference wavelength of approximate 690 nm in a microtiter plate reader (Bio-Rad, Hercules, Calif., USA). For each plate, one positive control was set as a calibrator in order to standardize between different runs.

Detailed Methods for BrdU Proliferation Assay and Flow Staining.

For proliferation assays, 10 μM of BrdU was added during the last 45 min of CD3+ T-cell activation. BrdU-pulsed CD3+ T-cells were harvested and stained with PE-conjugated anti-human-CD69 (BD Bioscience) to confirm TCR response. The surface stained cells were then fixed and permeabilized with BD Cytofix/Cytoperm buffer, treated with staining enhancer Cytoperm plus buffer and re-fixed, followed by incubation with DNase for 1 hour at 37° C. to expose the BrdU. After washing with cytoperm buffer, intracellular BrdU was stained with fluorescent anti-BrdU-FITC antibody for 20 min at room temperature. For live-dead discrimination, cells were stained with 4′-6-Diamidino-2-phenylindole (DAPI) 1 μg/ml. Finally, cells were resuspended with staining buffer that contained 3% Fetal Bovine Serum (FBS). The percentage of cells that incorporated BrdU and expressed CD69 was determined on a FACScan flow cytometer (BD Biosciences) using FlowJo software (BD Biosciences).

Detailed Methods for Measurement of TREC DNA in Purified T-Cells.

To detect sjTRECs, a real-time quantitative PCR method was used(4). Each PCR reaction was performed in triplicate in a 25-μl solution containing 250 ng DNA, 1.0× TaqMan Universal PCR master mix (Applied Biosystems, Branchburg, N.J.), 200 nM forward and reverse primer, and 250 nM probe. Quantification of TREC was normalized by GAPDH. GAPDH was detected in 100 ng of DNA using 300 nM of the forward primer, 900 nM of the reverse primer, and 875 nM of the probe. In addition to the samples, a five-point standard curve of sjTREC from 1×10 copies to 1×10 copies and a five-point standard curve of GAPDH from 1×10³ copies to 1×10⁷ copies were used to quantify the results. The standard curve was run in each plate to assess interassay variations in PCR efficiency. Quantitative PCR was performed by an ABI system (PerkinElmer), a standard curve was plotted, and TREC values were calculated by the ABI770 software. Samples were analyzed in triplicate and sjTREC levels expressed as copies per 250 ng of genomic DNA.

Detailed Methods for hTERT Promoter Sequencing.

Two primer pairs of 5′-TTTGTTAGCATTTCAGTGTTTGC-3′ (F1) (SEQ ID NO:5) and 5′-AAGGTGAAGGGGCAGGAC-3′(R1) (SEQ ID NO:6) and 5′-GTCCTGCCCCTTCACCTT-3′ (F2) (SEQ ID NO:7) and 5′-AGCACCTCGCGGTAGTGG-3′ (R2) (SEQ ID NO:8) using the Advantage-GC PCR kit (Clontech, USA). The PCR products were running on a 1% agarose gel and then cut each band from the gel separately. The final PCR products (1050 bp and 274 bp) were cloned into pCR2.1 vector (Invitrogen, Carlsbad, Calif., USA) via the TOPO TA cloning kits. Two resultant positive clones were sequenced by BigDye Terminator v3.1 Cycle in ABI PRISM 3130x/Genetic Analyzer.

SNP-A Methods.

Affymetrix Genome-Wide Human SNP Array 6.0 kits (Affymetrix, Santa Clara, Calif.) were used for analysis of chromosome 5 using DNA from purified CD3+ T-cells from 27 patients. A stringent algorithm was applied for identification of somatic SNP-A lesions: Changes reported in the inventors' internal (based on 1003 controls) or publicly-available copy number variant (CNV) databases were considered germline and excluded. Signal intensity was analyzed and SNP calls determined using Gene Chip Genotyping Analysis Software Version 4.0 (GTYPE). Results of Affymetrix 6.0 arrays were analyzed using Genotyping Console version 3.0 (Affymetrix). No CNV for UPDs were identified in chromosome 5 in T-cells from MDS patients.

### REFERENCES II

1. Pfaffl M W. A new mathematical model for relative quantification in real-time RT-PCR. Nucleic Acids Res. 2001 May 1; 29(9):e45.
2. Shen J, Terry M B, Gurvich I, Liao Y, Senie R T, Santella R M. Short telomere length and breast cancer risk: a study in sister sets. Cancer Res. 2007 Jun. 1; 67(11):5538-5544.
3. Rollison D E, Epling-Burnette P K, Park J Y, Lee J H, Park H, Jonathan K, Cole A L, Painter J S, Guerrier M, Melendez-Santiago J, Fulp W, Komrokji R, Lancet J, List A F. Telomere length in myelodysplastic syndromes. Leuk Lymphoma. 2011 Jun. 3.
4. Pongers-Willemse M J, Verhagen O J, Tibbe G J, Wijkhuijs A J, de Haas V, Roovers E, van der Schoot C E, van Dongen J J. Real-time quantitative PCR for the detection of minimal residual disease in acute lymphoblastic leukemia using junctional region specific TaqMan probes. Leukemia. 1998 December; 12(12):2006-2014.

### Example 8

### Clinical Characteristics of MDS Patients

Characteristics of the 35 MDS patients and 42 controls are summarized in Table 3. Of the 42 controls and 35 cases, 18 (43%) and 24 (69%) were male, respectively (p=0.02). MDS patients were classified as refractory anemia with or without ringed sideroblast (RA, RARS) (n=2, 5.7%), refractory cytopenia with multilineage dysplasia (RCMD) (n=15, 42.9%), refractory anemia with excess blasts (RAEB)-1 (n=5, 14.3%) and RAEB-2 (n=9, 25.7%) and MDS-unclassified (MDS-U, n=4, 11.4%) based on WHO classification criteria(14). Based on IPSS, 12 (34.3%) were low risk, 9 (25.7%) intermediate-1, 8 (22.9%) intermediate-2, and 6 (17.1%) high risk(1). Twenty of 35 (57.1%) had identifiable cytogenetic abnormalities by metaphase karyotyping or FISH, and 15 (42.9%) had normal cytogenetics.

### Example 9

### Age-Independent Telomere Attrition in Purified T-Cells in MDS

The mean relative telomere length in purified T-cells was compared relative to hemoglobin and found to be significantly shorter among MDS cases (n=35) (median=1.1, 95% CI, 1.1-1.4) compared to controls (n=42) (median=2.1, 95% CI, 1:9-2.3) after adjustment for age and sex (p<0.0001) (FIG. 1A) using log-transformed data. In healthy individuals, T-cell telomere length declined progressively with age (r=−0.447, p=0.003) (FIG. 1B) in agreement with previous reports reflecting the proliferative pressure on the T-cell compartment(20). Although not statistically significant, the age-related trend was similar in cases (r=−0.177, p=0.309) (FIG. 1B). To further evaluate the effect of age, the inventors compared telomere length between MDS cases and controls stratified into younger (<65 yrs) and older (>65 yrs) age groups (FIG. 1C). As expected, telomere lengths were significantly shorter among older controls compared to younger controls (p=0.001), although no difference in telomere lengths was observed between older and younger MDS cases. MDS cases tended to have shorter telomeres than controls in both the <65 age group (p=0.001) and the 65+ age group (p=0.003). In general, MDS patients within the younger group had telomeres that were shorter than the oldest healthy individuals attesting to the proliferative stress or loss of telomeric repair in the T-cell compartment.

CD3+ T-cells were purified by negative selection from blood of MDS patients (n=35) and healthy donors (n=42). Telomere length was detected using quantitative PCR with 293 T-cells were used as a calibrator, as previously described(16). Results were analyzed using the ΔΔCt method, as described herein. Case-control differences for the telomere lengths in CD3+ T-cells were compared using the Wilcoxon sum rank test. P-values for the case-control differences are shown at the top of each panel. Log-transformed values for telomere length were then used in multivariate logistic regression analyses to adjust for age (as continuous variable) and sex, and the case-control difference in telomere length in CD3+ T-cells was statistically significant (p<0.0001). Correlation between telomere length and age was assessed in cases and controls using the Spearman rank correlation coefficient. p<0.05 was considered statistically significant. FIG. 1A shows box and whisker-plots of telomere length in CD3+ T-cells in MDS patients compared to controls. Telomere length (y-axis) relative to hgb was inversely correlated with the age in CD3+ T-cells from cases and controls (FIG. 1B). (C) Age-stratified analysis among younger <65 years and older (≧65 y) (FIG. 1C). All tests were two-sided and associations were considered statistically significant at a significance level of p<0.05.

### Example 10

### Proliferative Defects in MDS T-Cells

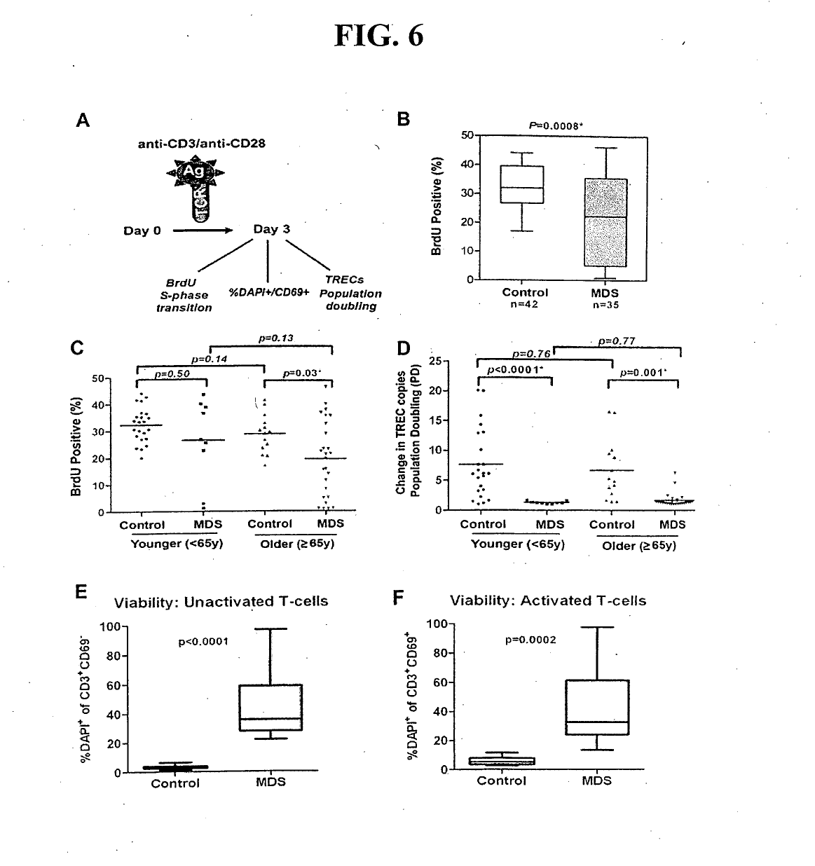

Replicative potential is closely linked to telomere length and telomerase activation^{(10, 21)}. To define the proliferative capacity of T-cells in MDS compared to healthy controls, both the percentage of cells capable of entering the cell cycle (ie, percentage of cells in S-phase as indicated by BrdU incorporation), cell death (percentage of CD69+DAPI+ cells) and their proliferative burst potential (ie, population doubling) were assessed after anti-CD3/CD28 bead stimulation (FIG. 6A). The percentage of BrdU positive T-cells is significantly reduced in MDS cases (median=22.3, 95% CI, 16.3-26.3) compared to controls (median=32.0, 95% CI, 30.0-34.6) (p=0.0008) (FIG. 6B). However, wide variability was observed in the percentage of BrdU positive cells in MDS. The discrepancy in cell cycle regulation was more relevant in older cases compared to younger cases (case:control difference, p=0.03 in older group compared to case:control difference p=0.50 in younger group), but there were patients in both age groups with normal capacity for S-phase transition (FIG. 6C).

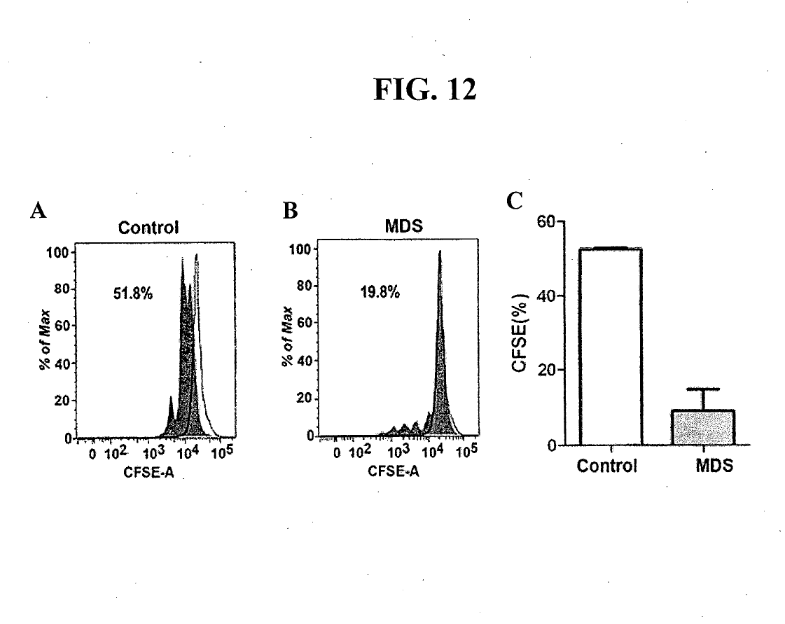

Telomerase deficiency primarily impacts replication burst potential as failure to induce the telomere repair machinery leads to apoptosis and premature growth arrest(6). Estimation of replication potential was assessed using sjTREC dilution (FIG. 6A) as a measure of population doubling (PD) after in vitro activation with, anti-CD3/CD28-coated beads. The change in copy number reflects the dilution of DNA through expansion after activation. Results were compared in younger and older cases and controls. In controls (n=42), the mean sjTREC DNA copy number decreased from 80 to 15 copies per cell after stimulation, indicating that an average of 6 PDs occurred in the three-day period of the assay. Fluorescent lipophilic dye 5,6-carboxyfluorescein diacetate succinimidyl ester (CFSE) staining confirmed that T-cell populations in controls progress through roughly 5-7 generations within the 3-day expansion period, which is consistent with the sjTREC dilution assay (FIGS. 12A-12C). In the CSFE assay, ˜50% of the cells are able to divide in bead-activated control T-cells. Compared to controls, PD was significantly decreased in MDS cases, both in the younger (p<0.0001) and older (p=0.001) groups (FIG. 6D). In MDS, expansion progressed past one generation of division on average in only 15% of cells using the CSFE assay (FIGS. 12A-12C). This indicates that there is a defect in the proliferative clonal burst in MDS T-cells after stimulation. Reduced clonal burst potential suggests that the cells undergo premature cell death or apoptosis. The % of DAPI positive cells prior to and after anti-CD3/CD28 stimulation was assessed in CD69+-activated T-cells. As shown in FIGS. 6E and 6F, cell death was significantly higher in activated T-cells from MDS patients compared to controls.

### Example 11

### Short Telomere Length in NaïVe T-Cells in MDS Patients Suggests Inherent Loss of Telomere Maintenance

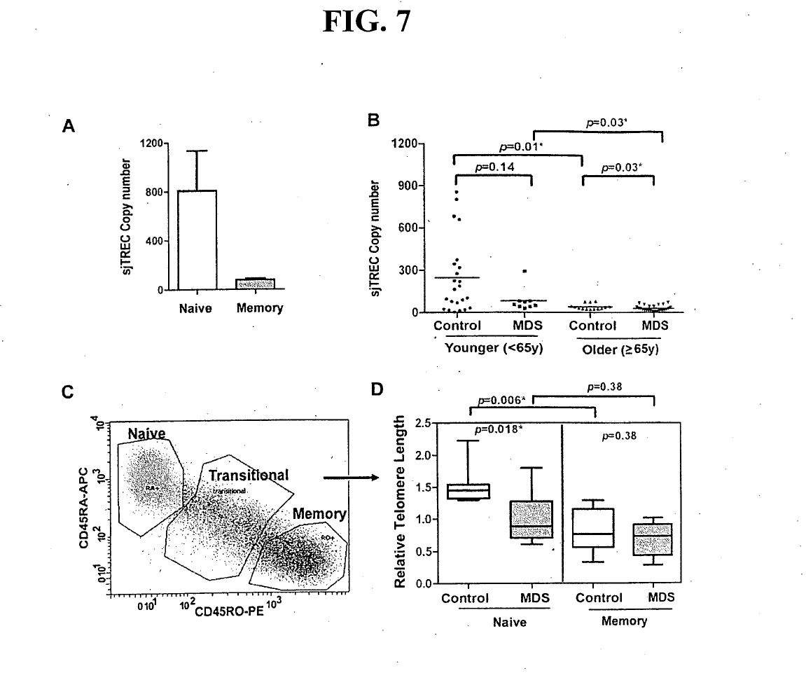

Telomeres may shorten secondary to proliferative stress, as previously hypothesized in MDS myeloid progenitors. In this case, telomere length should be shortest in memory cells that have been exposed to antigenic stimulation. To assess whether telomere dysfunction is a feature of antigen exposure, naïve and memory populations were compared in cases and controls. sjTREC expression was examined and found, as expected, to reside primarily within the naïve (CD45RA⁺CD45RO⁻) not the memory (CD45RO⁺CD45RA⁻) population (FIG. 7A)(17) in sorted populations, as shown in FIG. 3C. Using phenotyping, naïve T-cells have been shown to be reduced in MDS(19, 22, 23). Since naïve T-cells are lost through a normal aging process, sjTREC copy number was compared in unstimulated samples from younger and older groups of cases and controls (FIG. 7B). As indicated by sjTREC copies, MDS cases tended to have less naïve cells in the 65+ age group (p=0.01). Although decreased in younger MDS cases compared to younger controls, this difference was not statistically significant (p=0.14, Wilcoxon).

Flow sorting was then used to isolate naïve and memory cells, as shown in FIG. 7C. Purified (sorted) naïve and memory T-cells were compared in a subset of MDS cases (n=8) and healthy controls (n=8) with a sufficient number of cells available for sorting. This subgroup of cases and controls were individually age-matched. In healthy controls, the mean relative telomere length was significantly longer in naïve T-cells compared to memory cells (p=0.006) reflecting their history of proliferative expansion (FIG. 7D)(10). The telomere length in sorted naïve cells was significantly decreased among MDS cases compared to controls (p=0.018), whereas no case-control difference was observed for telomere length measured in memory cells. Thus, telomere length is shortest within the naïve T-cell compartment in MDS patients consistent with an inherent telomere defect. Since it is critical to confirm the naïve phenotype of the sorted MDS T-cells, additional surface markers including CD27, CD28, and CCR7(24) were used and indeed confirmed that the isolated naïve cells have no evidence of antigen-activation-associated phenotypic changes (data not shown). These results indicate that shorter telomere length in bulk T-cell populations reflects in part a reduction in naïve cells, as well as premature telomere attrition in antigen inexperienced, naïve T-cells indicating that the defect is not dependent on past antigen exposure.

### Example 12

### Impaired Inducible Telomerase Enzyme Activity in T-Cells in MDS Patients

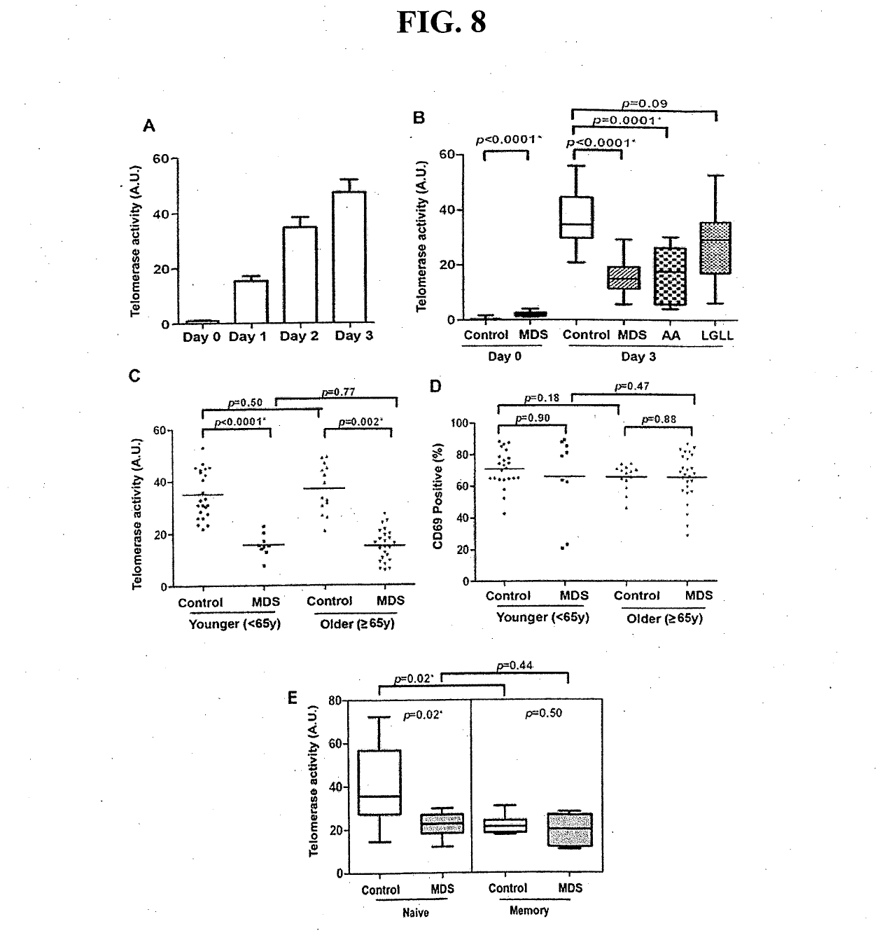

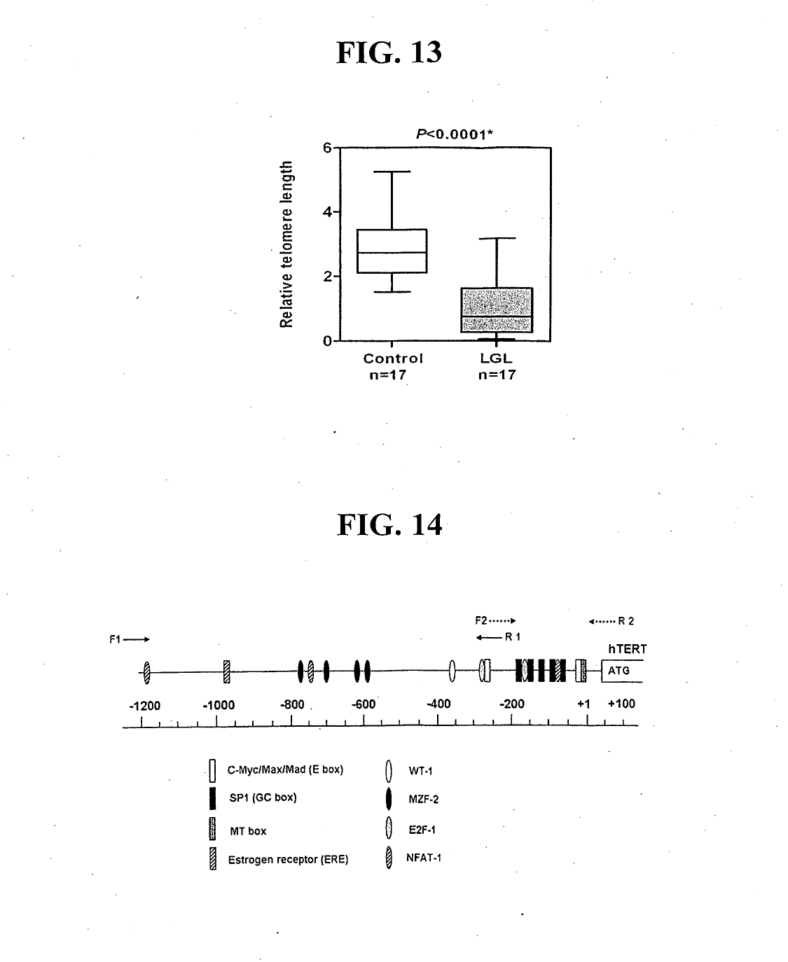

Telomeric repeats may be lost in the naïve compartment if there is a defect in telomere repair as there is an exorbitant demand for proliferation in the naïve compartment after thymic involution(21). In T-cells, telomerase activity is generally absent in resting cells and induced upon TCR activation(21). To assess the activity of telomerase in cases and controls, the TCR was stimulated with anti-CD3/anti-CD28-conjugated beads and the amount of enzyme activity determined by the TRAP assay. Preliminary assays were conducted to confirm the kinetics of telomerase activity and a 3-day stimulation period was found to be optimal (FIG. 8A). Results were compared at day 0 (ie, basal activity) and after 3 days of stimulation (ie, inducible activity) in cases and controls. Basal telomerase activity was significantly higher in MDS cases compared to controls, but the levels were still very low compared to that of stimulated cells (FIG. 8B): After stimulation, the amount of telomerase activity induced in MDS T-cells was significantly less (median=15.0, 95% CI, 13.4-17.2) than healthy controls (median=34.6, 95% CI, 33.6-39.3). (Wilcoxon p<0.0001) after adjustment for age and sex using log-transformed values of telomerase activity (p<0.0001). Aplastic anemia (AA) and MDS are clinically and epidemiologically linked bone marrow failure syndromes(6) and large granular lymphocyte (LGL) leukemia is related to MDS by virtue of an association with cytopenias and clonal T-lymphocyte expansion(11). As shown in FIG. 13, telomere length in purified T-cells from patients with LGL leukemia was significantly shorter than control. The inventors compared inducible telomerase activity in purified T-cells in LGL leukemia, MDS and aplastic anemia cases and found similar levels of activity in LGL leukemia and controls, as shown in FIG. 8B, but low telomerase activity in both of the highly-related bone marrow failure diseases.

The inventors then compared the telomerase activity in older and younger MDS case and control groups (FIG. 8C) and observed a significant difference in both in cases (p<0.0001 younger group and p=0.002 older group). No difference in telomerase activity was observed in the younger and older controls (p=0.50) indicating that telomerase function is preserved although telomere length shortens with age. To ensure that T-cells from patients were adequately responsive to TCR stimulation, the inventors measured the surface expression of CD69; an inducible early activation antigen(25). In contrast to the impairment in telomerase activity, BrdU incorporation (FIG. 6C), and population doubling (FIG. 6D), the expression of CD69 was similar in younger (p=0.90) and older (p=0.88) cases and controls on day 3 (FIG. 8D) suggesting that the telomerase defect is not due to a generalized loss in TCR signaling responses.

Given the data on telomere length in naïve and memory cells, telomerase activity was examined among these cell populations. Inducible telomerase activity was greatest in control naïve cells and significantly less in memory cells (p=0.02, FIG. 8E). Moreover, the case:control difference in telomerase activity was present in naïve cells (p=02) while the memory T-cell population showed no difference (p=0.50, FIG. 8E) compared to controls indicating that a primary telomerase deficiency underlies telomere loss in the naïve T-cell compartment and that the defect is not related to prior antigen exposure.

### Example 13

### hTERT Transcriptional Deficiency Responsible for Impaired Telomerase Function

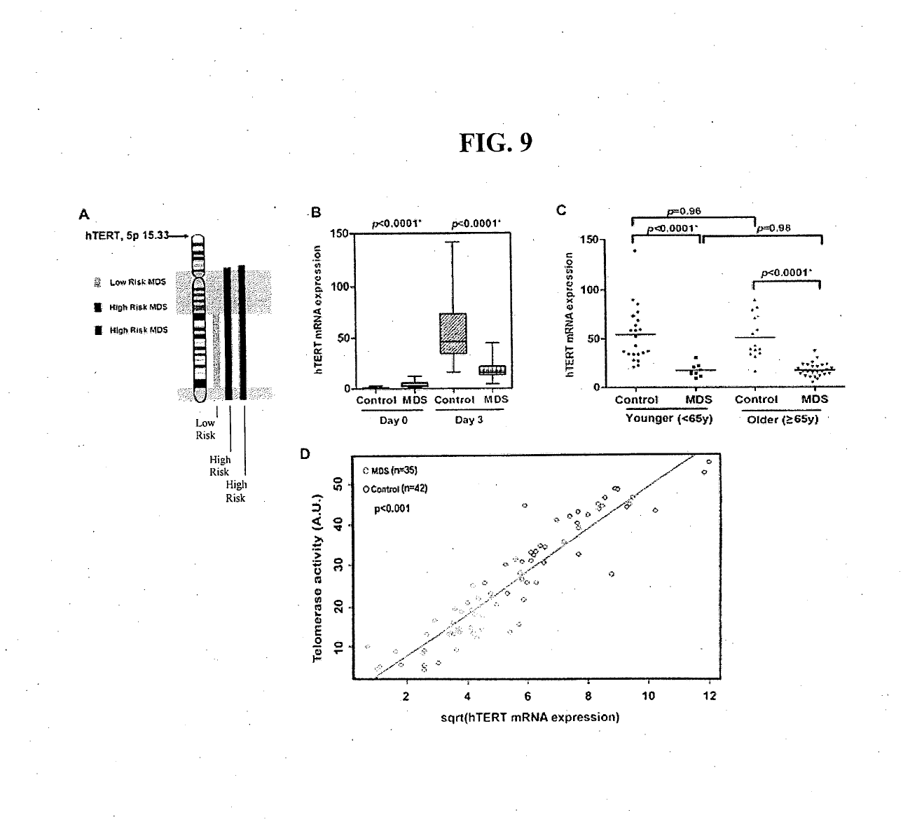

Transcriptional induction of hTERT mRNA represents the rate-limiting step for telomerase regulation(26). Interstitial deletions on chromosome 5 occur in MDS and refined mapping of the region was recently examined using SNP-A. Abnormalities including deletions and uniparental disomy (UPD) were reported to occur in 142 (12%) of 1,155 patients with MDS, MDS/myeloproliferative neoplasms, and AML(27). The hTERT gene maps to chromosome 5p15.33, which is non-overlapping with regions spanning commonly deleted regions on 5q (FIG. 9A). SNP-A was then performed on purified T-cells with data compared to an internal control series (n=1,003) and the Database of Genomic Variants. No CNV or UPD were detected on chromosome 5 in purified T-cells (data not shown). To assess transcriptional regulation, basal and inducible hTERT mRNA expression was examined on day 0 and 3 after stimulation. As expected in control cells, there is little to no hTERT mRNA without stimulation (FIG. 9B). Basal hTERT mRNA expression was higher in MDS cases (p<0.0001) compared to controls, but the inducible amount was significantly lower in cases (median=15.7, 95% CI, 14.9-20.5 vs. median=46.0, 95% CI, 46.8-64.6 in controls) (p<0.0001) (FIG. 9B) and this case-control difference was independent of age and sex using square-root transformed hTERT data. Comparing the younger and older patient and control subset, the hTERT deficiency was observed across both age groups (p<0.0001 in younger vs. older cases and controls, FIG. 9C).

The amount of inducible hTERT mRNA expression was then correlated to the level of inducible telomerase activity in T-cells, as shown in FIG. 9D using square-root transformation to normalize hTERT data (r=0.89, p<0.001). The results suggest that there is a mechanistic link between telomerase deficiency and hTERT transcriptional impairment as indicated by the close correlation between these two events.

### Example 14

### Sequencing of hTERT Promoter

There are three main regions required for induction of hTERT expression consisting of a sequence from −203 to +55, corresponding to the promoter core, an activating region −1397 and −798, and an inhibitory region between −798 and −400. Several transcription factor binding sites are located in these regions, as shown in FIG. 14. No mutations were observed by direct sequencing of cloned hTERT promoter DNA from five MDS patients with 4-5 clones tested per patient.

### Example 15

### No Correlation Between Clinical Classification and Telomere Repair

Measurements including telomere length, hTERT mRNA, and inducible telomerase activity were correlated among patients to IPSS score, WHO subtype and cytogenetics, as shown in Table 4. There was no statistically significant association between these telomere variables and disease stratification, although patients with higher-risk (int-2+ high) IPSS classification demonstrated a trend for shorter telomere length (p=0.1), lower induction of telomerase activity (p=0.06), and less hTERT mRNA expression (p=0.12) in T-cells.

### Example 16

### Proliferation Defect is Related to Telomerase Deficiency not Telomere Length

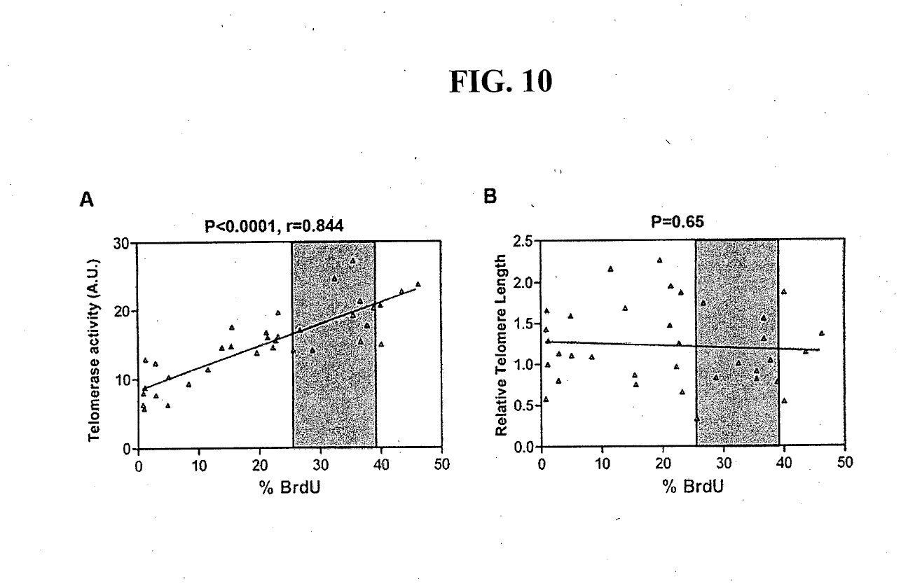

Telomere length must be critically short to force cell cycle arrest. It is possible that telomerase deficiency has important consequences that more broadly impact cell fate as treatment with RNA interference (siRNA) to hTERT resulted in an immediate suppression in replication potential and apoptosis without impacting telomere length⁸. The proportion of T-cells in MDS patients that underwent S-phase transition (i.e., % BrdU positive) was examined in relationship to telomerase activity and to telomere length. A mechanistic link between replication potential and telomerase activity is suggested by the close correlation (r=0.844, p<0.0001) between BrdU incorporation and telomerase enzymatic activity, but unrelated to telomere length (p=0.65) (FIGS. 10A and 10B).

Myelodysplastic syndromes (MDS) include a spectrum of age-related hematological neoplasms characterized by dysplasia, cytopenias and potential for Acute Myeloid Leukemia (AML) progression(1). Because complex mutations are acquired within the myeloid progenitor or stem cell pool, the biological characteristics are enormously heterogeneous(2, 3). The early manifestations of MDS, however, are relatively well conserved and include inflammation within the bone marrow microenvironment coupled with excessive proliferation and apoptosis of myeloid progenitors(4, 5). In MDS patients, telomere length is shortened by an unknown process related possibly to proliferative stress or by ineffective telomeric repair.

Telomeres are a repetitive hexanucleotide (TTAGGG) region that protect from chromosomal deterioration(6). T-cells and hematopoietic stem/progenitor cells need telomere replenishment by telomerase, which is a specialized reverse transcriptase composed of the rate-limiting telomerase reverse transcriptase (hTERT) enzyme, the Telomerase RNA Component (hTERC) template, and the shelterin stabilizing proteins. hTERT gene expression is transcriptionally regulated and its induction limits replicative senescence and protects regenerating somatic cells from apoptosis and genome instability(7). Mutations in telomere components have demonstrated the role for telomerase in hematopoietic repopulation and in immune homeostasis(8). hTERC, hTERT and mutations in shelterin proteins in patients with aplastic anemia and dyskaratosis congenital (DKC) have been informative to demonstrate the consequences of telomerase deficiency in bone marrow failure and predisposition to cancer(6, 9). In patients with mutations in telomere components, T-cells undergo TCR signal transduction but fail to display normal clone size after activation(6). In previous studies, siRNA silencing of hTERT in T-cells renders them sensitive to apoptosis and hinders their regeneration indicating a direct role for telomerase in replication potential(10). Compared to myeloid cells, there is no data regarding the length of telomeres or telomerase function within the T-cell compartment in MDS patients(2). Of all somatic cells, T-cells are dependent on telomere repair because of their high rate of turnover. This activity is particularly important for maintenance of naïve cells after age-dependent thymic involution(10). In MDS, the T-cells are characterized by skewed repertoire distribution(11) and form suppressive lymphoid aggregates within the bone marrow of some patients where they can directly suppress hematopoiesis(12, 13).

This study was conducted to examine the mechanism of MDS T-cell deregulation related to telomere function. The inventors report that deficiency in hTERT mRNA expression is important in MDS and reveal a novel mechanistic link between aplastic anemia and other primary telomere repair disorders.

Evidence indicates that accelerated telomere shortening occurs within the myeloid progenitor and stem cell compartments in patients with MDS(16, 28-32), while telomere length is preserved in stromal cells(33), suggesting that the defect is acquired within the hematopoietic compartment. Few studies have directly examined telomerase activity in MDS, and those that have were limited to unstimulated myeloid cells(29, 30). This study is the first to investigate the mechanism for pre-mature telomere attrition in MDS T-cells. For normal telomerase regulation, the inventors show the need for TCR stimulation and demonstrate how this process is disrupted in all MDS patients compared to controls.

Telomerase plays a complex role during tumorigenesis and in the regulation of normal homeostasis. Normal telomerase activity coupled to short telomeres in LGL leukemia and older control individuals suggests that telomere repair is an incomplete restorative process. Replication in MDS T-cells was closely correlated to the activity of telomerase rather than to telomere length indicating that telomerase broadly impacts cell fate. Telomere repair is predominantly functional within naïve cells so the telomerase deficiency selectively affected the naïve compartment and potentially contributed to reduced numbers of naïve T-cells. Oligoclonal memory expansion and limited repertoire diversity are prominent, unexplained features of the disease(23). In order to maintain the total number of T-cells in the periphery, expansion of memory clones may fill the void left by the declining naïve cell compartment. The inventors found evidence of excessive activation-induced cell death in MDS T-cells consistent with abnormalities in this pathway.

Naïve T-cells appear to prematurely reach replicative senescence possibly impacted by age and accelerated proliferation after thymic involution. A telomerase deficiency in naïve T-cells hinders their regeneration which may contribute to the accumulation of senescent cells in MDS. Some MDS patients have increased regulatory T-cells(34, 35), and enhanced liberation of inflammatory cytokines such as tumor necrosis factor-alpha (TNF-α)(36, 37), Fas ligand(4, 38), and other proapoptotic cytokines that may impact hematopoiesis and T-cell function (3, 5). Since senescent T-cells remain viable but produce more inflammatory cytokines and demonstrate non-MHC-restricted cytotoxicity through acquired natural-killer receptors(39), the telomere defect in naïve cells may contribute indirectly to the inflammatory cytokine milieu in MDS.

Telomerase activity in MDS correlates with the amount of hTERT mRNA and these results are consistent with a deficiency in hTERT mRNA production. Although interstitial deletions of chromosome 5 occur in this disease(27), UPD and deletions in the region of the hTERT gene on 5p were not detected in T-cells. Signaling events leading to the transcriptional induction of CD69 expression are intact indicating that the hTERT defect is selective. The minimal core promoter (located at −40 to −775) is required for expression and has a NFAT1-binding site flanked by two SP binding sites(26), as determined by luciferase and electrophoretic mobility shift assays (EMSA) using serial deletions of the 5′-UTR of the hTERT gene. Mutations in the core promoter region of the 5′-hTERT UTR were not detected in MDS patients (FIG. 14). Defects in telomere repair have been reported in inflammatory diseases such as rheumatoid arthritis (RA) and the defect is manifest by hTERT transcriptional repression(10, 40). In cancer and normal cells, mediators of hTERT transcription include cAMP responsive element binding protein (CREB), estradiol (E2) estrogen receptor alpha and beta (ERα and ERβ) c-Myc, β-catenin/TCF4, HIF-1, signal transducers and activators of transcription (STAT)-3 and interferon regulatory factor 1 (IRF-1)(41, 42). These transcription factors act downstream of well defined signaling cascades such as Ras, PI3K/Akt, NF-KB, MAP kinase and GSK-3β(41). At this point, It is unclear which pathways are blocked in MDS T-cells. Examination of hTERT deficient T-cells in RA demonstrated that apoptosis induction is dependent on upregulation of DNA damage sensing enzymes DNA-dependent protein kinase (DNA-PKcs), activation of pro-apoptotic BH3-only proteins Bim/Bmf, and activation of the MAPK family member JNK, but was independent of pATM and p53 (43). Increased expression of Bim/Bmf would be expected to induce apoptosis by stimulating the release of pro-apoptotic Bcl-2-family proteins like Bax/Bak resulting in the activation of downstream caspases(44). Pharmacological inhibition of JNK with TLK199, a glutathione analog that inhibits JNK kinase activity, is now under investigation in MDS(45). Stabilization of apoptosis through inhibition of this pathway should be further verified. A strong case can be made for a mechanism involving suppression of hTERT through changes in specific signaling events that limits the cellular of yield of T-cells by inducing cell death.

While multiple congenital and acquired mutations have been reported in hTERT, hTERC, and shelterin proteins(6, 46-48); this is the first report implicating aberrant inducible hTERT transcriptional regulation in MDS or in bone marrow failure. Telomere length in peripheral blood leukocytes from MDS patients was independent of genotype in samples screened for hTERT polymorphisms and mutations in codons 202, 279, 305, 412, 441 and 1062(16). Codons 1062, 279 and 412 polymorphisms were previously shown to be more prevalent in aplastic anemia(16, 47). Generally, myeloid and lymphoid cells in MDS are considered genetically divergent populations with the clonal population arising exclusively within a multipotent myeloid progenitor that gives rise to abnormal granulocytes, megakaryocytes and erythrocytes(49). A telomere defect within both the myeloid and lymphoid populations could be explained if the disease initiating event originates within a pluripotent stem cell with myeloid and lymphoid populating capacity. The origin of MDS within a true stem cell population is a matter of debate(49). Especially in MDS myeloid cells, critically short telomeres may be recognized as DNA damage resulting in the recruitment and activation of the DNA-damage response pathway, (i.e., DNA-PKcs or p53 pathway) stimulating apoptosis and increasing genetic events(50). According to studies in solid tumors and in patients with congenital or acquired mutations in telomere components, individuals with shorter telomeres are at higher risk, for developing malignancies due to genomic instability(32).

The current study defines MDS as a member of the telomere repair disorders. Opportunities to improve MDS diagnosis and to design novel therapeutic interventions may be achieved from a better understanding of telomere abnormalities in T-cells and in the myeloid hematopoietic/stem cell compartment contributing to malignant transformation in MDS.

TABLE 3
Clinical characteristics of MDS cases and controls
Characteristics of Cases and Controls

| Age | Mean age (range) | | p-value |
|---|---|---|---|
| Case: Controls | | | |
| Controls (n = 42) | 52 (17-84) | | |
| MDS Cases (n = 35) | 67 (28-87) | | 0.1 |
| Younger Case: Control group (<65 y)* | | | |
| Controls (n = 23) | 47 (26-63) | | |
| MDS Cases (n = 9) | 52 (28-61) | | 0.26 |
| Older Case: Control group (≧65 y) | | | |
| Controls (n = 14) | 73 (65-84) | | 0.97 |
| MDS Cases (n = 26) | 73 (65-87) | | |
| Sex (Male/Female) | N (M/F) | % (M/F) | |
| Controls (n = 42) | 24/11 | 69/31 | |
| MDS Cases (n = 35) | 18/24 | 43/57 | 0.02 |

| Clinical Characteristics of MDS Cases | n | % |
|---|---|---|
| IPSS** classification | | |
| Low | 12 | 34.3 |
| Intermediate-1 | 9 | 25.7 |
| Intermediate-2 | 8 | 22.9 |
| High | 6 | 17.1 |
| Cytogenetics | | |
| Normal | 15 | 42.9 |
| Abnormal | 20 | 57.1 |
| WHO^{##} MDS subtype | | |
| RA^{#} with ringed sideroblasts (RARS) | 2 | 5.7 |
| RCMD^{$} | 15 | 42.9 |
| RA with excess blasts (RAEB)-1^{%} | 5 | 14.3 |
| RAEB-2 | 9 | 25.7 |
| MDS-unclassified (MDS-U) | 4 | 11.4 |

*Within the younger groups, subset statistical analyses were also conducted excluding the 5 youngest controls with an age range that more closely approximated cases as described in the text.
**IPSS = International Prognostic Scoring System;
^{##}WHO = World Health Organization;
^{#}RA = Refractory anemia
^{$}RCMD = Refractory cytopenia with multilineage dysplasia including patients with (n = 2) or without ringed sideroblasts.
^{%}Refractory anemia with excess blasts-1 and 2.

TABLE 4
Clinical characteristics and telomerase measurements in CD3+ T-cells
myelodysplastic syndrome (MDS) cases.

| N = 35 | | | RTL* | | hTERT^{S} | | TA^{#} | |
|---|---|---|---|---|---|---|---|---|
| Characteristic | n | % | mean (SD) | p-value | mean (SD) | p-value | mean (SD) | p-value |
| IPSS** score | | | | | | | | |
| Low + int-1 | 21 | 60 | 1.08 (0.10) | | 14.42 (1.53) | | 14.43 (1.31) | |
| Int-2 + high | 14 | 40 | 1.36 (0.13) | 0.10 | 10.45 (1.97) | 0.12 | 10.96 (1.14) | 0.06 |
| Cytogenetics | | | | | | | | |
| Normal | 15 | 43 | 1.07 (0.08) | | 12.72 (2.00) | | 12.59 (1.36) | |
| Abnormal | 20 | 57 | 1.36 (0.12) | 0.07 | 13.38 (1.71) | 0.80 | 13.62 (1.35) | 0.60 |
| WHO^{##} MDS | | | | | | | | |
| subtype | | | | | | | | |
| MDSU + RARS | 6 | 17 | 1.45 (0.23) | | 17.39 (4.95) | | 13.05 (2.85) | |
| RCMD | 15 | 43 | 1.11 (0.09) | | 12.58 (1.72) | | 13.74 (1.53) | |
| RAEB1 + RAEB2 | 14 | 40 | 1.25 (1.46) | 0.33 | 11.76 (1.62) | 0.31 | 12.55 (1.35) | 0.85 |

*RTL = Relative telomere length;
^{S}hTERT = Human telomerase reverse transcriptase
^{#}TA = Telomerase activity;
**IPSS = International Prognostic Scoring System;
^{##}WHO = World Health Organization

### REFERENCES III

1. Greenberg P, Cox C, LeBeau M M, Fenaux P, Morel P, Sanz G, et al. International scoring system for evaluating prognosis in myelodysplastic syndromes. Blood. 1997 Mar. 15; 89(6):2079-2088.
2. Bejar R, Stevenson K, Abdel-Wahab O, Galili N, Nilsson B, Garcia-Manero G, et al. Clinical effect of point mutations in myelodysplastic syndromes. N Engl J Med. 2011 Jun. 30; 364(26):2496-2506.
3. Calado RT. Immunologic aspects of hypoplastic myelodysplastic syndrome. Semin Oncol. 2011 October; 38(5):667-672.
4. Lepelley P, Grardel N, Erny O, Iaru T, Obein V, Cosson A, et al. Fas/APO-1 (CD95) expression in myelodysplastic syndromes. Leuk Lymphoma. 1998 July; 30(3-4):307-312.
5. Raza A, Alvi S, Borok R Z, Span L, Parcharidou A, Alston D, et al. Excessive proliferation matched by excessive apoptosis in myelodysplastic syndromes: the cause-effect relationship. Leuk Lymphoma. 1997 September; 27(1-2): 111-118.
6. Young NS. Telomere biology and telomere diseases: implications for practice and research. Hematology Am Soc Hematol Educ Program. 2010; 2010:30-35.
7. Ju Z, Zhang J, Gao Y, Cheng T. Telomere dysfunction and cell cycle checkpoints in hematopoietic stem cell aging. Int J Hematol. 2011 July; 94(1):33-43.
8. Calado R T, Young N S. Telomere maintenance and human bone marrow failure. Blood. 2008 May 1; 111(9):4446-4455.
9. Young N S. Pathophysiologic mechanisms in acquired aplastic anemia. Hematology Am Soc Hematol Educ Program. 2006:72-77.
10. Fujii H, Shao L, Colmegna I, Goronzy J J, Weyand C M. Telomerase insufficiency in rheumatoid arthritis. Proc Natl Acad Sci USA. 2009 Mar. 17; 106(11):4360-4365.
11. Wlodarski M W, Gondek L P, Nearman Z P, Plasilova M, Kalaycio M, Hsi E D, et al. Molecular strategies for detection and quantitation of clonal cytotoxic T-cell responses in aplastic anemia and myelodysplastic syndrome. Blood. 2006 Oct. 15; 108(8):2632-2641.
12. Sloand E M, Kim S, Fuhrer M, Risitano A M, Nakamura R, Maciejewski J P, et al. Fas-mediated apoptosis is important in regulating cell replication and death in trisomy 8 hematopoietic cells but not in cells with other cytogenetic abnormalities. Blood. 2002 Dec. 15; 100(13):4427-4432.
13. Sloand E M, Melenhorst J J, Tucker Z C, Pfannes L, Brenchley J M, Yong A, et al. T-cell immune responses to Wilms tumor 1 protein in myelodysplasia responsive to immunosuppressive therapy. Blood. 2011 Mar. 3; 117(9):2691-2699.
14. Germing U, Strupp C, Kuendgen A, Isa S, Knipp S, Hildebrandt B, et al. Prospective validation of the WHO proposals for the classification of myelodysplastic syndromes. Haematologica. 2006 December; 91(12):1596-1604.
15. Cawthon R M, Smith K R, O'Brien E, Sivatchenko A, Kerber R A. Association between telomere length in blood and mortality in people aged 60 years or older. Lancet. 2003 Feb. 1; 361(9355):393-395.
16. Rollison D E, Epling-Burnette P K, Park J Y, Lee J H, Park H, Jonathan K, et al. Telomere length in myelodysplastic syndromes. Leuk Lymphoma. 2011 Jun. 3.
17. McFarland R D, Douek D C, Koup R A, Picker L J. Identification of a human recent thymic emigrant phenotype. Proc Natl Acad Sci USA. 2000 Apr. 11; 97(8):4215-4220.
18. Richardson M W, Sverstiuk A, Hendel H, Cheung T W, Zagury J F, Rappaport J. Analysis of telomere length and thymic output in fast and slow/non-progressors with HIV infection. Biomed Pharmacother. 2000 February; 54(1):21-31.
19. McDaniel J M, Zou J X, Fulp W, Chen D T, List A F, Epling-Burnette P K. Reversal of T-cell tolerance in myelodysplastic syndrome through lenalidomide immune modulation. Leukemia. 2012 June; 26(6):1425-1429.
20. Poulin J F, Viswanathan M N, Harris J M, Komanduri K V, Wieder E, Ringuette N, et al. Direct evidence for thymic function in adult humans. J Exp Med. 1999 Aug. 16; 190(4):479-486.
21. Goronzy J J, Fujii H, Weyand C M. Telomeres, immune aging and autoimmunity. Exp Gerontol. 2006 March; 41(3):246-251.
22. Maciejewski J P, Hibbs J R, Anderson S, Katevas P, Young N S. Bone marrow and peripheral blood lymphocyte phenotype in patients with bone marrow failure. Exp Hematol. 1994 October; 22(11):1102-1110.
23. Zou J X, Rollison D E, Boulware D, Chen D T, Sloand E M, Pfannes L V, et al. Altered naive and memory CD4+ T-cell homeostasis and immunosenescence characterize younger patients with myelodysplastic syndrome. Leukemia. 2009 July; 23(7):1288-1296.
24. Campbell J J, Murphy K E, Kunkel E J, Brightling C E, Soler D, Shen Z, et al. CCR7 expression and memory T cell diversity in humans. J Immunol. 2001 Jan. 15; 166(2):877-884.
25. Amlot P L, Tahami F, Chinn D, Rawlings E. Activation antigen expression on human T cells. I. Analysis by two-colour flow cytometry of umbilical cord blood, adult blood and lymphoid tissue. Clin Exp Immunol. 1996 July; 105(1):176-182.
26. Chebel A, Rouault J P, Urbanowicz I, Baseggio L, Chien W W, Salles G, et al. Transcriptional activation of hTERT, the human telomerase reverse transcriptase, by nuclear factor of activated T cells. J Biol Chem. 2009 Dec. 18; 284(51):35725-35734.
27. Jerez A, Gondek L P, Jankowska A M, Makishima H, Przychodzen B, Tiu R V, et al. Topography, clinical, and genomic correlates of 5q myeloid malignancies revisited. J Clin Oncol. 2012 Apr. 20; 30(12):1343-1349.
28. Boultwood J, Fidler C, Kusec R, Rack K, Elliott P J, Atoyebi O, et al. Telomere length in myelodysplastic syndromes. Am J Hematol. 1997 December; 56(4):266-271.
29. Counter C M, Gupta J, Harley C B, Leber B, Bacchetti S. Telomerase activity in normal leukocytes and in hematologic malignancies. Blood. 1995 May 1; 85(9):2315-2320.
30. Gurkan E, Tanriverdi K, Baslamisli F. Telomerase activity in myelodysplastic syndromes. Leuk Res. 2005 October; 29(10):1131-1139.
31. Sashida G, Ohyashiki J H, Nakajima A, Sumi M, Kawakubo K, Tauchi T, et al. Telomere dynamics in myelodysplastic syndrome determined by telomere measurement of marrow metaphases. Clin Cancer Res. 2003 April; 9(4):1489-1496.
32. Lange K, Holm L, Vang Nielsen K, Hahn A, Hofmann W, Kreipe H, et al. Telomere shortening and chromosomal instability in myelodysplastic syndromes. Genes Chromosomes Cancer. 2010 March; 49(3):260-269.
33. Marcondes A M, Bair S, Rabinovitch P S, Gooley T, Deeg H J, Risques R. No telomere shortening in marrow stroma from patients with MDS. Ann Hematol. 2009 July; 88(7):623-628.
34. Kordasti S Y, Afzali B, Lim Z, Ingram W, Hayden J, Barber L, Matthews K, et al. IL-17-producing CD4(+) T cells, pro-inflammatory cytokines and apoptosis are increased in low risk myelodysplastic syndrome. Br J Haematol. 2009 April; 145(1):64-72.
35. Kordasti S Y, Ingram W, Hayden J, Darling D, Barber L, Afzali B, et al. CD4+CD25high Foxp3+ regulatory T cells in myelodysplastic syndrome (MDS). Blood. 2007 Aug. 1; 110(3):847-850.
36. Campioni D, Secchiero P, Corallini F, Melloni E, Capitani S, Lanza F, et al. Evidence for a role of TNF-related apoptosis-inducing ligand (TRAIL) in the anemia of myelodysplastic syndromes. Am J Pathol. 2005 February; 166(2):557-563.
37. Sawanobori M, Yamaguchi S, Hasegawa M, Inoue M, Suzuki K, Kamiyama R, et al. Expression of TNF receptors and related signaling molecules in the bone marrow from patients with myelodysplastic syndromes. Leuk Res. 2003 July; 27(7):583-591.
38. Brazil J J, Gupta P. Constitutive expression of the Fas receptor and its ligand in adult human bone marrow: a regulatory feedback loop for the homeostatic control of hematopoiesis. Blood Cells Mol Dis. 2002 July-August; 29(1):94-103.
39. Henel G, Singh K, Cui D, Pryshchep S, Lee W W, Weyand C M, et al. Uncoupling of T-cell effector functions by inhibitory killer immunoglobulin-like receptors. Blood. 2006 Jun. 1; 107(11):4449-4457.
40. Schonland S O, Lopez C, Widmann T, Zimmer J, Bryl E, Goronzy J J, et al. Premature telomeric loss in rheumatoid arthritis is genetically determined and involves both myeloid and lymphoid cell lineages. Proc Natl Acad Sci USA. 2003 Nov. 11; 100(23):13471-13476.
41. Daniel M, Peek G W, Tollefsbol T O. Regulation of the human catalytic subunit of telomerase (hTERT). Gene. 2012 May 1; 498(2):135-146.
42. Zhang Y, Toh L, Lau P, Wang X. Telomerase reverse transcriptase (TERT) is a novel target of Wnt/beta-catenin pathway in human cancer. J Biol Chem. 2012 Jul. 31.
43. Shao L, Goronzy J J, Weyand C M. DNA-dependent protein kinase catalytic subunit mediates T-cell loss in rheumatoid arthritis. EMBO Mol Med. 2010 October; 2(10):415-427.
44. Kuroda J, Taniwaki M. Involvement of BH3-only proteins in hematologic malignancies. Crit Rev Oncol Hematol. 2009 August; 71(2):89-101.
45. Gore S D, Hermes-DeSantis ER. Future directions in myelodysplastic syndrome: newer agents and the role of combination approaches. Cancer Control. 2008 October; 15 Suppl: 40-49.
46. Fibbe W E. Telomerase mutations in aplastic anemia. N Engl J Med. 2005 Apr. 7; 352(14):1481-1483.
47. Yamaguchi H, Calado R T, Ly H, Kajigaya S, Baerlocher G M, Chanock S J, et al. Mutations in TERT, the gene for telomerase reverse transcriptase, in aplastic anemia. N Engl J Med. 2005 Apr. 7; 352(14):1413-1424.
48. Ly H, Calado R T, Allard P, Baerlocher G M, Lansdorp P M, Young N S, et al. Functional characterization of telomerase RNA variants found in patients with hematologic disorders. Blood. 2005 Mar. 15; 105(6):2332-2339.
49. Nimer S D. MDS: a stem cell disorder—but what exactly is wrong with the primitive hematopoietic cells in this disease? Hematology Am Soc Hematol Educ Program. 2008:43-51.
50. Briatore F, Barrera G, Pizzimenti S, Toaldo C, Casa C D, Laurora S, et al. Increase of telomerase activity and hTERT expression in myelodysplastic syndromes. Cancer Biol Ther. 2009 May; 8(10):883-889.

1. A method for detecting myelodysplastic syndrome (MDS) in a subject, comprising analyzing a biological sample from the subject for telomerase function, wherein an impaired telomerase function is indicative of MDS in the subject.

2. The method according to claim 1, wherein said analyzing comprises determining one or more of the following: TERT transcription (TERT mRNA expression), telomere length, telomerase activity, TERC RNA template, telomerase induction (inducible TERT level), and T-cell receptor excision circles (TREC).

3. The method according to claim 1, further comprising obtaining the biological sample from the subject.

4. The method according to claim 1, wherein the biological sample is whole blood, plasma, or serum.

5. The method according to claim 1, wherein the biological sample comprises peripheral blood T cells.

6. The method according to claim 1, wherein the biological sample comprises CD3+ T cells, and said analyzing comprises analyzing telomerase function in the CD3+ T cells.

7. The method according to claim 1, wherein the MDS is one or more of among refractory cytopenia with unilineage dysplasia (refractory anemia, refractory neutropenia, or refractory thrombocytopenia); refractory anemia with sideroblasts (SARS); refractory anemia with sideroblasts-thrombocytosis (SARS-t); refractory cytopenia with multileneage dysplasia (RCMD) (refractory cytopenia with multilineage dysplasia and ring sideroblasts (RCMD-RS), including subjects with pathological changes not restricted to red cells (i.e., prominent white cell precursor and platelet precursor (megakaryocyte) dysplasia); refractory anemia with excess blasts (RAEB)-I or -II; acute leukemia; myelodysplastic-myeloproliferative overlap syndrome; 5q-syndrome; unclassifiable myelodysplasia; or refractory cytopenia of childhood (dysplasia in childhood).

8. The method according to claim 1, wherein the MDS is refractory cytopenia with multilineage dysplasia (RCMD), refractory anemia with excess blasts-1 (RAEB-1), or refractory anemia with excess blasts-2 (RAEB-2).

9. (canceled)

10. The method according to claim 1, wherein said analyzing comprises measuring telomerase function in the biological sample and comparing the measured level of telomerase function to a reference level of telomerase function.

11. (canceled)

12. The method according to claim 1, wherein the subject has previously been treated for MDS, and telomerase function is analyzed to monitor the status of the MDS in the subject and/or effectiveness of the treatment.

13. A method for treatment of myelodysplastic syndrome (MDS) in a subject determined to have MDS, comprising administering a therapeutic treatment to the subject, wherein the subject has been determined to have MDS based on impaired telomerase function.

14. The method according to claim 13, wherein said method comprises qualitatively or quantitatively analyzing a biological sample from the subject for telomerase function, wherein an impaired telomerase function is indicative of the presence of MDS in the subject; and treating the subject if the subject is determined to have MDS.

15. The method according to claim 14, wherein said analyzing comprises determining one or more of the following: TERT transcription (TERT mRNA expression), telomere length, telomerase activity, TERC RNA template, telomerase induction (inducible TERT level), and T-cell receptor excision circles (TREC).

16-21. (canceled)

22. A method for monitoring myelodysplastic syndrome (MDS) in a subject post-treatment, comprising administering a treatment for the MDS to the subject; and analyzing a biological sample from the subject for telomerase function, wherein the biological sample is obtained from the subject post-treatment, and wherein an impaired telomerase function in the biological sample is indicative of MDS in the subject.

23. A composition comprising an array or panel for detection of myelodysplastic syndrome (MDS), comprising: (a) one or more moieties that can bind specifically to one or more proteins, or nucleic acids encoding said one or more proteins, whose expression is associated with telomerase function.

24. The composition of claim 23, further comprising: (b) one or more moieties that can bind specifically to one or more proteins, or nucleic acids encoding said one or more proteins, whose expression is associated with the presence of a malignancy.

25. The composition of claim 23, wherein said one or more moieties of (a) and/or (b) comprises one or more moieties selected from among an antibody, or an antigen binding fragment of said antibody, a peptide, a nucleic acid, or a ligand.

26. The composition of claim 24, wherein the malignancy is a hematologic malignancy.

27. The composition of claim 26, wherein the hematologic malignancy is MDS.

28. The composition of claim 23, further comprising a substrate, wherein the one or more moieties of (a) and (b) are attached to said substrate.

29-30. (canceled)
